SEQUENCE LISTING (25 sequences)

SEQ ID NO: 1 - protein, 287 aa, Artificial Sequence
  130-402 of bovine; A1,4-galactosyltransferase I catalytic domain with Y289L, C342T mutation and a N-terminal tag
MASMTGGQQMGRGSSLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 2 - protein, 273 aa, Artificial Sequence
  bovine  A1,4-galactosyltransferase I catalytic domain (130-402) with Y289L and  C342T mutation
SLTACPEESPLLVGPMLIEFNIPVDLKLVEQQNPKVKLGGRYTPMDCISPHKVAIIIPFRNRQEHLKYWLYYLHPILQRQQLDYGIYVINQAGESMFNRAKLLNVGFKEALKDYDYNCFVFSDVDLIPMNDHNTYRCFSQPRHISVAMDKFGFSLPYVQLFGGVSALSKQQFLSINGFPNNYWGWGGEDDDIYNRLAFRGMSVSRPNAVIGKTRMIRHSRDKKNEPNPQRFDRIAHTKETMLSDGLNSLTYMVLEVQRYPLYTKITVDIGTPS

SEQ ID NO: 3 - protein, 1006 aa, Artificial Sequence
  37-995 of Streptococcus pyogenes Endoglycosidase S with a N terminal tag
MHHHHHHSSGLVPRGSGMKETAAAKFERQHMDSPDLGTGGGSGIEGREEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 4 - protein, 959 aa, Artificial Sequence
  37-995 of Streptococcus pyogenes Endoglycosidase S
EEKTVQVQKGLPSIDSLHYLSENSKKEFKEELSKAGQESQKVKEILAKAQQADKQAQELAKMKIPEKIPMKPLHGPLYGGYFRTWHDKTSDPTEKDKVNSMGELPKEVDLAFIFHDWTKDYSLFWKELATKHVPKLNKQGTRVIRTIPWRFLAGGDNSGIAEDTSKYPNTPEGNKALAKAIVDEYVYKYNLDGLDVDVEHDSIPKVDKKEDTAGVERSIQVFEEIGKLIGPKGVDKSRLFIMDSTYMADKNPLIERGAPYINLLLVQVYGSQGEKGGWEPVSNRPEKTMEERWQGYSKYIRPEQYMIGFSFYEENAQEGNLWYDINSRKDEDKANGINTDITGTRAERYARWQPKTGGVKGGIFSYAIDRDGVAHQPKKYAKQKEFKDATDNIFHSDYSVSKALKTVMLKDKSYDLIDEKDFPDKALREAVMAQVGTRKGDLERFNGTLRLDNPAIQSLEGLNKFKKLAQLDLIGLSRITKLDRSVLPANMKPGKDTLETVLETYKKDNKEEPATIPPVSLKVSGLTGLKELDLSGFDRETLAGLDAATLTSLEKVDISGNKLDLAPGTENRQIFDTMLSTISNHVGSNEQTVKFDKQKPTGHYPDTYGKTSLRLPVANEKVDLQSQLLFGTVTNQGTLINSEADYKAYQNHKIAGRSFVDSNYHYNNFKVSYENYTVKVTDSTLGTTTDKTLATDKEETYKVDFFSPADKTKAVHTAKVIVGDEKTMMVNLAEGATVIGGSADPVNARKVFDGQLGSETDNISLGWDSKQSIIFKLKEDGLIKHWRFFNDSARNPETTNKPIQEASLQIFNIKDYNLDNLLENPNKFDDEKYWITVDTYSAQGERATAFSNTLNNITSKYWRVVFDTKGDRYSSPVVPELQILGYPLPNADTIMKTVTTAKELSQQKDKFSQKMLDELKIKEMALETSLNSKIFDVTAINANAGVLKDCIEKRQLLKK

SEQ ID NO: 5 - protein, 561 aa, Artificial Sequence
  1-344 of Lactobacillus casei  A1,6-fucosidase fused with a CPD tag and a his tag in the C-terminus
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANRALADGKILHNQNVNSWGPITVTPTTDGGETRFDGQIIVQMENDPVVAKAAANLAGKHAESSVVVQLDSDGNYRVVYGDPSKLDGKLRWQLVGHGRDHSETNNTRLSGYSADELAVKLAKFQQSFNQAENINNKPDHISIVGCSLVSDDKQKGFGHQFINAMDANGLRVDVSVRSSELAVDEAGRKHTKDANGDWVQKAENNKVSLSWDAQGHHHHHH

SEQ ID NO: 6 - protein, 344 aa, Lactobacillus casei
MNDNVAWFKQAKYGMMIHWGLYSLLAGEYRGESSSAYAEWIQSKFQIPNAEYGNLATAFNPLYFDAKKIVALAKQCGMQYLVVTTKHHDGFAMYHSKVDAYNVYDATPFHRDIIGELAEACQKAGLKFGLYYSQDLDWHDPNGGGYKSNDVETAGTTWDNSWDFPDEDQKNFDLCFDNKILPQIKEIMSNYGDIATAWFDVPMTLSEAQSQTIYDTVRELQPNCLINSRLGNGKYDFVSLGDNEIPKNKEDMNKTDVDYNEITGFKPSPLGLYETAGTINDSWGFSYHDQNWKTPRTLYRYKQHLNDFGINYLLNVGLDPLGRVPMMAEENLLAAKALEDEANR

SEQ ID NO: 7 - protein, 321 aa, Artificial Sequence
  40-359 of human  A1,3 fucosyltrasferase 6(GenBank Accession NO.AAD33509.1) with a N termina G
GDPTVYPNGSRFPDSTGTPAHSIPLILLWTWPFNKPIALPRCSEMVPGTADCNITADRKVYPQADAVIVHHREVMYNPSAQLPRSSRRQGQRWIWFSMESPSHCWQLKAMDGYFNLTMSYRSDSDIFTPYGWLEPWSGQPAHPPLNLSAKTELVAWAVSNWGPNSARVRYYQSLQAHLKVDVYGRSHKPLPQGTMMETLSRYKFYLAFKNSLHPDYITEKLWRNALEAWAVPVVLGPSRSNYERFLPPDAFIHVDDFQSPKDLARYLQELDKDHARYLSYFRWRETLRPRSFSWALAFCKACWKLQEESRYQTRGIAAWFT

SEQ ID NO: 8 - protein, 214 aa, Artificial Sequence
  Trastuzumab light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 450 aa, Artificial Sequence
  Trastuzumab heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  hRS7 light chain
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, Artificial Sequence
  hRS7  heavy chain
VQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 7 aa, Artificial Sequence
  Helicobacter pylori  A1,3 fucosyltransferase heptad repeat region
DDLRVNY

SEQ ID NO: 13 - protein, 364 aa, Helicobacter pylori
  misc_feature (169)..(169): Xaa = any of amino acids
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLXAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISI

SEQ ID NO: 14 - protein, 364 aa, Helicobacter pylori
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISI

SEQ ID NO: 15 - protein, 364 aa, Artificial Sequence
  1-364 of Helicobacter pylori 26695  A1,3 fucosyltransferase (GenBank Accession NO. AAD07710.1) with a C169S mutation
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLSAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISI

SEQ ID NO: 16 - protein, 371 aa, Artificial Sequence
  1-364 of Helicobacter pylori 26695  A1,3 fucosyltransferase (GenBank Accession NO. AAD07710.1) with 1 HPR
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNY

SEQ ID NO: 17 - protein, 379 aa, Artificial Sequence
  1-364 of Helicobacter pylori 26695  A1,3 fucosyltransferase (GenBank Accession NO. AAD07710.1) with 1 HPR and a C terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYLEHHHHHH

SEQ ID NO: 18 - protein, 378 aa, Artificial Sequence
  1-364 of Helicobacter pylori 26695  A1,3 fucosyltransferase (GenBank Accession NO. AAD07710.1) with 2 HPR
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNY

SEQ ID NO: 19 - protein, 386 aa, Artificial Sequence
  1-364 of Helicobacter pylori 26695  A1,3 fucosyltransferase (GenBank Accession NO. AAD07710.1) with 2 HPR and a C terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYLEHHHHHH

SEQ ID NO: 20 - protein, 378 aa, Artificial Sequence
  1-364 of Helicobacter pylori 26695  A1,3 fucosyltransferase (GenBank Accession NO. AAD07710.1) with 2 HPR and a C169S mutation
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLSAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNY

SEQ ID NO: 21 - protein, 386 aa, Artificial Sequence
  1-364 of Helicobacter pylori 26695  A1,3 fucosyltransferase (GenBank Accession NO. AAD07710.1) with C 169S mutation, 2 HPR and a C terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLSAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYLEHHHHHH

SEQ ID NO: 22 - protein, 385 aa, Artificial Sequence
  1-364 of Helicobacter pylori 26695  A1,3 fucosyltransferase (GenBank Accession NO. AAD07710.1) with 3 HPR
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNY

SEQ ID NO: 23 - protein, 393 aa, Artificial Sequence
  1-364 of Helicobacter pylori 26695  A1,3 fucosyltransferase (GenBank Accession NO. AAD07710.1) with 3 HPR and a C terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYLEHHHHHH

SEQ ID NO: 24 - protein, 392 aa, Artificial Sequence
  1-364 of Helicobacter pylori 26695  A1,3 fucosyltransferase (GenBank Accession NO. AAD07710.1) with 4 HPR
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNY

SEQ ID NO: 25 - protein, 400 aa, Artificial Sequence
  1-364 of Helicobacter pylori 26695  A1,3 fucosyltransferase (GenBank Accession NO. AAD07710.1) with 4 HPR and a C terminal His tag
MFQPLLDAFIESASIEKMVSKSPPPPLKIAVANWWGDEEIKEFKKSVLYFILSQRYAITLHQNPNESSDLVFSNPLGAARKILSYQNTKRVFYTGENESPNFNLFDYAIGFDELDFNDRYLRMPLYYAHLHYEAELVNDTTAPYKLKDNSLYALKKPSHHFKENHPNLCAVVNDESDLLKRGFASFVASNANAPMRNAFYDALNSIEPVTGGGSVRNTLGYKVGNKSEFLSQYKFNLCFENSQGYGYVTEKILDAYFSHTIPIYWGSPSVAKDFNPKSFVNVHDFNNFDEAIDYIKYLHTHPNAYLDMLYENPLNTLDGKAYFYQDLSFKKILDFFKTILENDTIYHNNPFIFYRDLHEPLISIDDLRVNYDDLRVNYDDLRVNYDDLRVNYLEHHHHHH

(19) United States
(12) Patent Application Publication
(10) Pub. No.: US 2025/0222124 A1
(43) Pub. Date: Jul. 10, 2025

(54) PROTEIN CONJUGATES WITH MULTIPLE PAYLOADS AND METHODS FOR MAKING THE SAME

(71) Applicant: GLYCO-THERAPY BIOTECHNOLOGY CO., LTD., Hangzhou, Zhejiang (CN)

(72) Inventors: Yi YANG, Hangzhou, Zhejiang (CN); Jiangping HU, Hangzhou, Zhejiang (CN); Zhentao SONG, Hangzhou, Zhejiang (CN)

(73) Assignee: GLYCO-THERAPY BIOTECHNOLOGY CO., LTD., Hangzhou, Zhejiang (CN)

(21) Appl. No.: 18/728,603

(22) PCT Filed: Jan. 27, 2022
(86) PCT No.: PCT/CN2022/074199

§ 371 Date: Jul. 12, 2024

(51) Int. Cl. A61K 47/68; C12N 9/10; C12P 21/00

(52) U.S. Cl. CPC A61K 47/68037; A61K 47/68031; A61K 47/6889; C12N 9/1051; C12P 21/005; C12Y 204/01

(57) ABSTRACT

Provides herein is a protein conjugate with multiple payloads and methods for making the same.

### BACKGROUND OF THE INVENTION

Antibody-drug conjugates (ADC) have been considered as promising drug candidates, as they enable targeted delivery of effective drug payloads, providing significantly improved therapeutic index. However, development of effective and safe ADCs remains challenging as many factors, including the chosen of the antibodies and the payloads, the linkage stability, the number and site of the installed payloads, and the homogeneity of the conjugates are difficult to control. Current nonspecific conjugation methods such as cysteine or lysine conjugation yield heterogeneous mixtures of products that differ in the sites and stoichiometry of modification, resulting in heterogenous pharmacological properties, Although several approaches have been developed to generate site-specific, homogeneous and stable ADCs, the majority of these methods still requires antibodies to be genetically modified either by site-directed mutations or by the introduction of genetically encoded tags that remains to be a laborious task and may compromise the yield of the antibodies. Therefore, genetic-engineering-free approaches that enable site-specific drug conjugation to antibodies would bring much-needed advance to the field.

Through a metabolic incorporating strategy, Okeley N. M. et al. were able to incorporate 6-thiofucose onto IgG glycans with a percentage of 60-70% (Okeley N. M. et al., Bioconjugate Chem. 2013, 24, 1650-1655). Following conjugation with a maleimide-linked MMAE yielded a conjugate with an average DAR of 1.3. However, the incorporation ratio of the unnatural 6-thiofucose were difficult to control, leading to heterogeneous antibodies conjugates.

Zhou Q. et al. using galactosyltransferases and sialyltransferases to introduce terminal sialic acids on the native glycans of N297 on the antibody (Zhou Q. et al., Bioconjugate Chem. 2014, 25, 510-520). Periodate oxidation of these sialic acids yielded aldehyde groups which were subsequently used to conjugate aminooxy functionalized cytotoxins via oxime ligation. This strategy enables the incorporation of an average of 1.6 cytotoxins per antibody molecule. Similarly, by treating co-fucosylated IgG with sodium periodate, Neri D. et al. were able to oxidize the core-fucose residues to an aldehyde, which were further used to prepare hydrazone conjugates with fluorophores and a dolastatin analogue (Neri D. et al., Chem. Commun., 2012, 48, 7100-7102). However, the use of periodate oxidation may lead to oxidative damage to the antibodies.

On the other hand, combination therapies have become increasingly necessary to overcome multidrug resistance. However, controlled site-specific conjugation of different or multiple payloads onto a single antibody molecule remains difficult to achieve using currently available strategies.

For example, due to the limited tolerance of enzymes towards the ribonucleotide-sugar derivatives and challenges in the synthesis of multiple-payload-bearing ribonucleotide-sugar derivatives, it has been very difficult to introduce multiple payloads to an antibody.

Some strategies relied on a glyco-transfer step to introduce an azido group to an antibody, followed by reacting with a bicyclo [6.1.0]nonyne (BCN) modified molecule bearing two active molecules to obtain a dual-conjugated protein. However, such molecules tend to be highly hydrophobic, and the reaction procedure usually needs the addition of organic solvents which are prone to induce aggregations in solutions.

Accordingly, methods for developing ADCs with multiple types of payloads is highly needed.

### SUMMARY OF THE INVENTION

The present disclosure provides a protein conjugate with multiple payloads (e.g. comprising at least two active molecules (e.g., functional groups or pharmaceutically active molecule)) on one conjugation site and a method for making the same.

The protein conjugate of the present disclosure has at least one of the following characteristics: (a) well-controlled and defined conjugation sites on the oligosaccharide of the protein; (b) well defined active-molecule-to-antibody-ratio (MAR); (c) at least two active molecules conjugated to one site of the oligosaccharide of the protein; (d) high homogeneity; (e) high stability (for example, the conjugation linkage between the Fuc* and the GlcNAc of Formula (I) is stable in the plasma (e.g. human plasma) for at least 1 day (e.g., at least two days, three days, four days, five days, six days, seven days, eight days or longer), as measured with mass spectrometry analysis or ELISA); (f) capable of binding to an antigen, with a similar binding affinity as the corresponding antibody; (g) capable of participating in a bioorthogonal ligation reaction; (h) capable of inhibiting tumor growth and/or tumor cell proliferation.

The present disclosure also provides a method for directly conjugating multiple active molecules to a protein (e.g. an antibody comprising a Fc fragment) at one conjugation site, by using α-1,3-fucotrasferases and a Q-Fuc* bearing two or more active molecules.

With the method of the present disclosure, various combinations of active molecules (e.g., azido group and tetrazinyl group, alkynyl group and tetrazinyl group, azido group and azido group, azido group and cytotoxin, cytotoxin and cytotoxin, or cytotoxin and agonist) could be transferred to the same site of a protein (e.g., an antibody).

The present disclosure also provides synthesis of the Q-Fuc* bearing two or more active molecules and use of these Q-Fuc* in preparing protein conjugates.

In one aspect, the present disclosure provides a protein conjugate, which comprises a protein and an oligosaccharide comprising a structure of Formula (I):

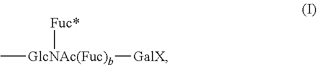

wherein: said GlcNAc is directly or indirectly linked to an amino acid of said protein, said GalX is an optionally substituted galactose, said Fuc is a fucose, and b is 0 or 1, said Fuc* is a fucose derivative comprising two or more active molecules (AM).

In some embodiments of the protein conjugate, the Fuc* comprises the structure of Formula (II):

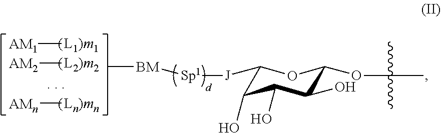

J is a jointer and is directly linked to the

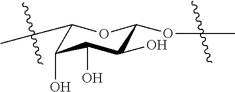

of Formula (II); Sp¹ is a spacer moiety, d is 0 or 1; BM is a branching moiety; L₁ to Lₙ each independently is a linker, m₁ to mₙ each independently is 0 or 1; AM₁ to AMₙ each independently is an active molecule; and n is an integer from 2-10.

In some embodiments of the protein conjugate, the J has a structure of:

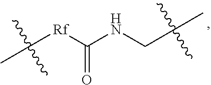

wherein R_{f} is —CH₂—, —NH— or —O—, and the right side of the structure is directly linked to the

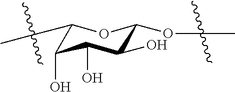

### of Formula (II).

In some embodiments of the protein conjugate, the J is

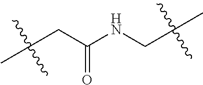 [SMILES: *CNC(=O)C*]

and the right side of the structure is directly linked to the

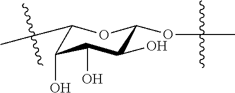

### of Formula (II).

In some embodiments of the protein conjugate, the BM comprises

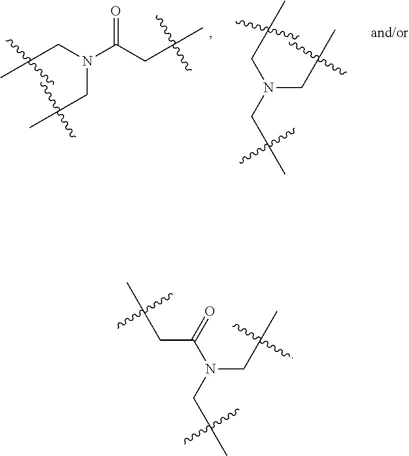 [SMILES: *CC(=O)N(C*)C* ; *CN(C*)C* ; *CC(=O)N(C*)C*]

In some embodiments of the protein conjugate, n is 2, and said Fuc* comprises the structure of Formula (III):

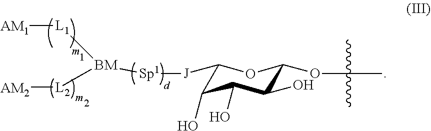

In some embodiments of the protein conjugate, the BM is selected from the group consisting of:

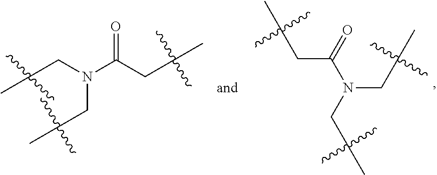 [SMILES: *CC(=O)N(C*)C* ; *CC(=O)N(C*)C*]

the right side of the structure is directly linked to said Sp¹ or said J.

In some embodiments of the protein conjugate, the Sp¹ is selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs², wherein each Rs¹ is independently selected from the group consisting of halogen, —OH, —NH₂ and —COOH, and each Rs² is independently selected from the group consisting of —O—, —S—,

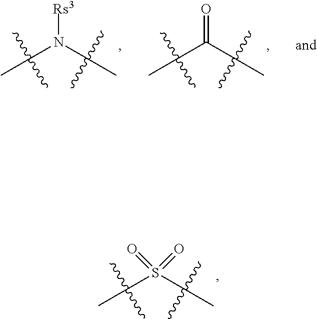

Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the protein conjugate, the Sp¹ is selected from the group consisting of:

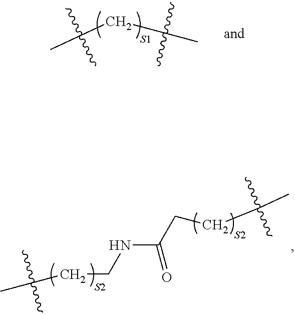

said S1 is an integer from 1-50, each said S2 is independently an integer from 0-50, each said —CH₂—(—CH₂— in the parentheses) independently optionally replaced by —O— with the proviso that two or more consecutive —CH₂— are not simultaneously replaced by —O—, the right side of the structure is linked to said J and the left side of the structure is linked to said BM. Sometimes, the —CH₂— may also be referred to as CH₂.

In some embodiments of the protein conjugate, each of L₁ to Lₙ is independently a linker of Formula (IV): (CL)_{y}-(FL)ₓ (IV), FL is a spacer moiety, x is 0 or 1; CL is a cleavable linker, y is 0 or 1; the right side of Formula (IV) is linked to said BM and the left side Formula (IV) is linked to said AM.

In some embodiments of the protein conjugate, the FL is a spacer moiety selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs², wherein each Rs¹ is independently selected from the group consisting of halogen, —OH, —NH₂ and —COOH, and each Rs² is independently selected from the group consisting of —O—, —S—,

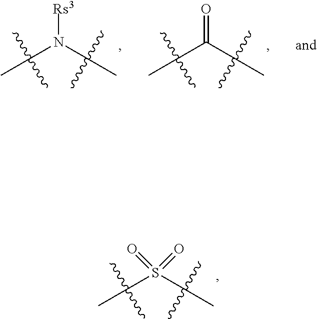

Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the protein conjugate, the FL is a spacer moiety selected from the group consisting of:

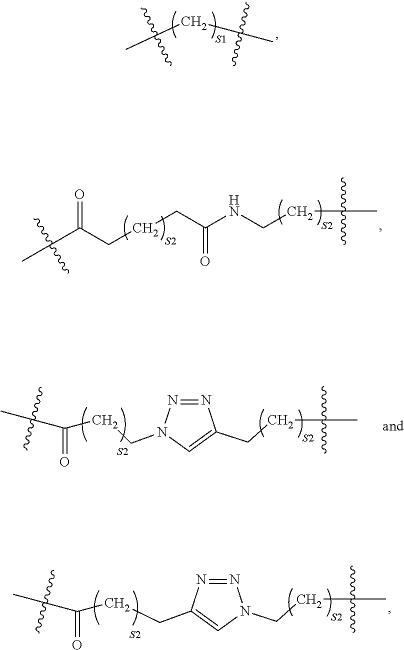

wherein said S1 is an integer from 1-50, each said S2 is independently an integer from 0-50, each said —CH₂—(—CH₂— in the parentheses) is independently optionally replaced by —O— with the proviso that two or more consecutive —CH₂— are not simultaneously replaced by —O—, the right side of the structure is linked to said BM, and the left side of the structure is linked to said CL or said AM.

In some embodiments of the protein conjugate, the CL is an acid-labile linker, a redox-active linker, a photo-active linker and/or a proteolytically cleavable linker.

In some embodiments of the protein conjugate, the CL is a vc-PAB-linker and/or a GGFG-linker.

In some embodiments of the protein conjugate, each of AM₁ to AMₙ is independently a chemically active molecule, an enzymatically active molecule, a biologically active molecule, and/or a pharmaceutically active molecule.

In some embodiments of the protein conjugate, the AM₁ to AMₙ independently comprises a chemically active molecule or enzymatically active molecule X_{F}.

In some embodiments of the protein conjugate, the chemically or enzymatically active molecule X_{F} comprises a functional moiety capable of participating in a ligation reaction.

In some embodiments of the protein conjugate, the X_{F} comprises a functional moiety capable of participating in a bioorthogonal ligation reaction.

In some embodiments of the protein conjugate, the X_{F} comprises a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof:

In some embodiments of the protein conjugate, the X_{F} comprises a functional moiety selected from the group consisting of:

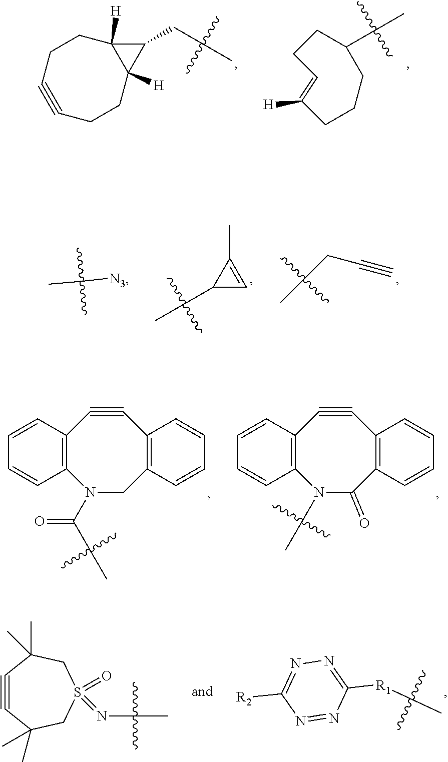

wherein R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group.

In some embodiments of the protein conjugate, the X_{F} comprises a functional moiety selected from the group consisting of:

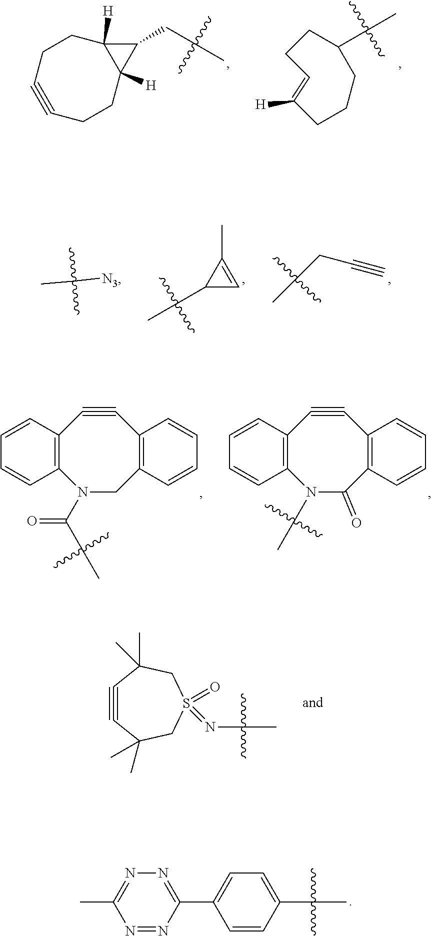

In some embodiments of the protein conjugate, the AM₁ to AMₙ independently comprises a biologically active molecule and/or a pharmaceutically active molecule P_{F}.

In some embodiments of the protein conjugate, the P_{F} comprises a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof:

In some embodiments of the protein conjugate, the P_{F} is a pharmaceutically active molecule.

In some embodiments of the protein conjugate, the P_{F} comprises a cytotoxin, an agonist, an antagonist, an antiviral agent, an antibacterial agent, an oligonucleotide, a polypeptide or any combination thereof:

In some embodiments of the protein conjugate, the P_{F} comprises a cytotoxin or an agonist.

In some embodiments of the protein conjugate, the P_{F} comprises a DNA or RNA damaging agent, an RNA polymerase inhibitor, a topoisomerase inhibitor and/or a microtubule inhibitor.

In some embodiments of the protein conjugate, the P_{F} comprises a pyrrolobenzodiazepine, auristatin, maytansinoids, duocarmycin, tubulysin, enediyene, doxorubicin, pyrrole-based kinesin spindle protein inhibitor, calicheamicin, amanitin, camptothecin and/or derivatives thereof:

In some embodiments of the protein conjugate, the P_{F} comprises a MMAE, a DXd, T785 and/or functional derivatives thereof:

In some embodiments of the protein conjugate, the GalX is linked to said GlcNAc through a β1,4 linkage.

In some embodiments of the protein conjugate, the GalX is a galactose.

In some embodiments of the protein conjugate, the GalX is a substituted galactose, and the hydroxyl group at one or more positions selected from the C2 position, the C3 position, the C4 position and the C6 position of the galactose, is substituted.

In some embodiments of the protein conjugate, the GalX is a substituted galactose, wherein the hydroxyl group at the C2 position of the galactose is substituted.

In some embodiments of the protein conjugate, the GalX is a monosaccharide.

In some embodiments of the protein conjugate, the GalX is substituted by

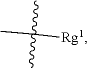

and said Rg¹ is selected from the group consisting of hydrogen, halogen, —NH₂, —SH, —N₃, —COOH, —CN, C₁-C₂₄ alkyl, C₃-C₂₄ cycloalkyl, C₂-C₂₄ alkenyl, C₅-C₂₄ cycloalkenyl, C₂-C₂₄ alkynyl, C₆-C₂₄ cycloalkynyl, C₂-C₂₄ (hetero)aryl, C₃-C₂₄ alkyl(hetero)aryl, C₃-C₂₄ (hetero)arylalkyl and any combination thereof, wherein each of said alkyl, cycloalkyl, alkenyl, cycloalkenyl, alkynyl, cycloalkynyl, (hetero)aryl, alkyl(hetero)aryl and (hetero)arylalkyl is independently optionally substituted by one or more Rs⁴ and/or is independently optionally interrupted by one or more Rs⁵, wherein each Rs⁴ is independently selected from the group consisting of halogen, —OH, —NH₂, —SH, —N₃, —COOH and —CN, and each Rs⁵ is independently selected from the group consisting of —O—, —S—,

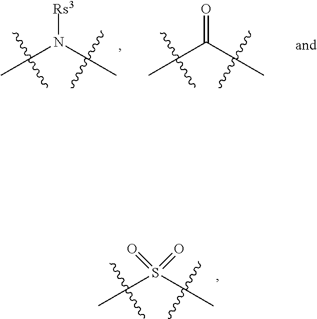

and Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the protein conjugate, the GalX is substituted by

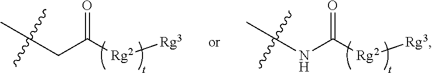

wherein t is 0 or 1, Rg² is selected from the group consisting of C₁-C₂₄ alkylene, C₃-C₂₄ cycloalkylene, C₂-C₂₄ alkenylene, C₅-C₂₄ cycloalkenylene, C₂-C₂₄ alkynylene, C₆-C₂₄ cycloalkynylene, C₂-C₂₄ (hetero)arylene, C₃-C₂₄ alkyl(hetero)arylene and C₃-C₂₄ (hetero)arylalkylene, wherein each of said alkylene, cycloalkylene, alkenylene, cycloalkenylene, alkynylene, cycloalkynylene, (hetero)arylene, alkyl(hetero)arylene and (hetero)arylalkylene is independently optionally substituted by one or more Rs⁴ and/or is independently optionally interrupted by one or more Rs⁵, Rg³ is selected from the group consisting of hydrogen, halogen, —OH, —NH₂, —SH, —N₃, —COOH, —CN, C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl, C₂-C₂₄(hetero)aryl and any combination thereof, wherein each of said C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl and C₂-C₂₄ (hetero)aryl is independently optionally substituted by one or more Rs⁴, each Rs⁴ is independently selected from the group consisting of halogen, —OH, —NH₂, —SH, —N₃—COOH and —CN and each Rs⁵ is independently selected from the group consisting of —O—, —S—,

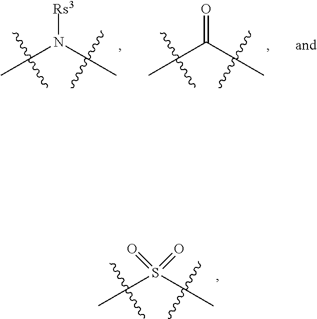

wherein Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the protein conjugate, the GalX comprises a chemically active molecule and/or enzymatically active molecule X_{G}.

In some embodiments of the protein conjugate, the X_{G} comprises a functional moiety capable of participating in a ligation reaction.

In some embodiments of the protein conjugate, the X_{G} comprises a functional moiety capable of participating in a bioorthogonal ligation reaction.

In some embodiments of the protein conjugate, the X_{G} comprises a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof:

In some embodiments of the protein conjugate, the X_{G} comprises a

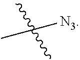 [SMILES: *N=[N+]=[N-]]

In some embodiments of the protein conjugate, the GalX is selected from the group consisting of:

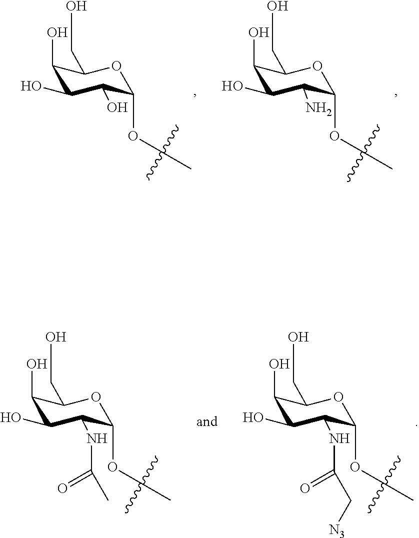

In some embodiments of the protein conjugate, the protein comprises an antigen binding fragment and/or an Fc fragment.

In some embodiments of the protein conjugate, the oligosaccharide is an N-linked oligosaccharide.

In some embodiments of the protein conjugate, the oligosaccharide is linked to an Asparagine (Asn) residue of said protein.

In some embodiments of the protein conjugate, the GlcNAc of Formula (I) is directly linked to an Asn residue of said protein.

In some embodiments of the protein conjugate, the GlcNAc of Formula (I) is linked to a saccharide of said oligosaccharide.

In some embodiments of the protein conjugate, the GlcNAc of Formula (I) is linked to a mannose of said oligosaccharide, and optionally b is 0.

In some embodiments of the protein conjugate, the protein comprises a Fc fragment, and said oligosaccharide is linked to said Fc fragment.

In some embodiments of the protein conjugate, the protein comprises a Fc fragment, and said oligosaccharide is linked to the CH2 domain of said Fc fragment.

In some embodiments of the protein conjugate, the protein comprises a Fc fragment, and said oligosaccharide is linked to the Asn297 of said Fc fragment, numbered according to the Kabat numbering system.

In some embodiments of the protein conjugate, the protein is an antibody.

In some embodiments of the protein conjugate, the protein is a monoclonal antibody.

In some embodiments of the protein conjugate, the protein is an IgG antibody.

In some embodiments of the protein conjugate, the protein is a humanized antibody.

In some embodiments of the protein conjugate, said Fuc* is linked to said GlcNAc through an α1,3 linkage.

In some embodiments of the protein conjugate, b is 1, and said Fuc is linked to said GlcNAc through an α1,6 linkage.

In some embodiments of the protein conjugate, n is 2, said Fuc* comprises the AM₁ and the AM₂, both said AM₁ and said AM₂ comprises a X_{F}, the X_{F} of AM₁ and the X_{F} of AM₂ are identical or different.

In some embodiments of the protein conjugate, n is 2, said Fuc* comprises the AM₁ and the AM₂, both said AM₁ and said AM₂ comprises a X_{F}, the X_{F} of AM₁ and the X_{F} of AM₂ is independently selected from the group consisting of:

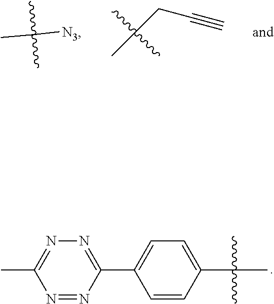

In some embodiments of the protein conjugate, the X_{F} of AM₁ does not react bioorthogonally with said X_{F} of AM₂.

In some embodiments of the protein conjugate, n is 2, said Fuc* comprises the AM₁ and the AM₂, the AM₁ comprises a X_{F} and the AM₂ comprises a P_{F}, or the AM₁ comprises a P_{F} and the AM₂ comprises a X_{F}.

In some embodiments of the protein conjugate, n is 2, said Fuc* comprises the AM₁ and the AM₂, both said AM₁ and said AM₂ comprises a P_{F}, the P_{F} of AM₁ and the P_{F} of AM₂ are identical or different.

In some embodiments of the protein conjugate, the protein conjugate of the present disclosure comprises 1-20 of said structure of:

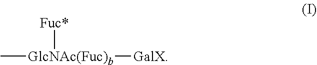

In some embodiments of the protein conjugate, the protein conjugate of the present disclosure comprises 2 or 4 of said structure of:

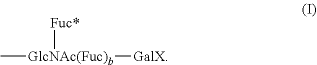

In some embodiments of the protein conjugate, the protein conjugate of the present disclosure comprises 2 of said structure of:

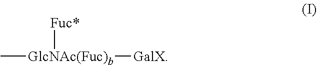

In some embodiments of the protein conjugate, the protein conjugate of the present disclosure has a structure of Formula (V):

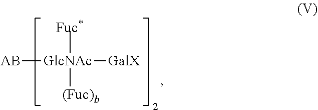

wherein AB is an antibody comprising a Fc fragment or a Fc-fusion protein, the GlcNAc is directly linked to an Asn of the Fc fragment of the AB, the Fuc is linked to the GlcNAc through an α1,6 linkage, the GalX is linked to the GlcNAc through a β1,4 linkage, the Fuc* is linked to the GlcNAc through an α1,3 linkage and b is 0 or 1.

In some embodiments of the protein conjugate, the protein conjugate of the present disclosure comprises 4 of said structure of:

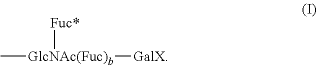

In some embodiments of the protein conjugate, the protein conjugate of the present disclosure has a structure of Formula (VI):

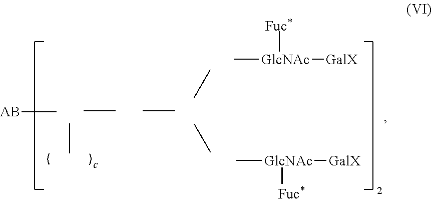

wherein AB is an antibody comprising a Fc fragment or a Fc-fusion protein, [symbol] is a GlcNAc, [symbol] is a mannose, [symbol] is a fucose linked to the [symbol] through an α1,6 linkage, c is 0 or 1; said oligosaccharide is linked to an Asn of the Fc fragment of the AB through the [symbol] , the GalX is linked to the GlcNAc through a β1,4 linkage, and the Fuc* is linked to the GlcNAc through an α1,3 linkage.

In some embodiments of the protein conjugate, the Fuc* is selected from the group consisting of:

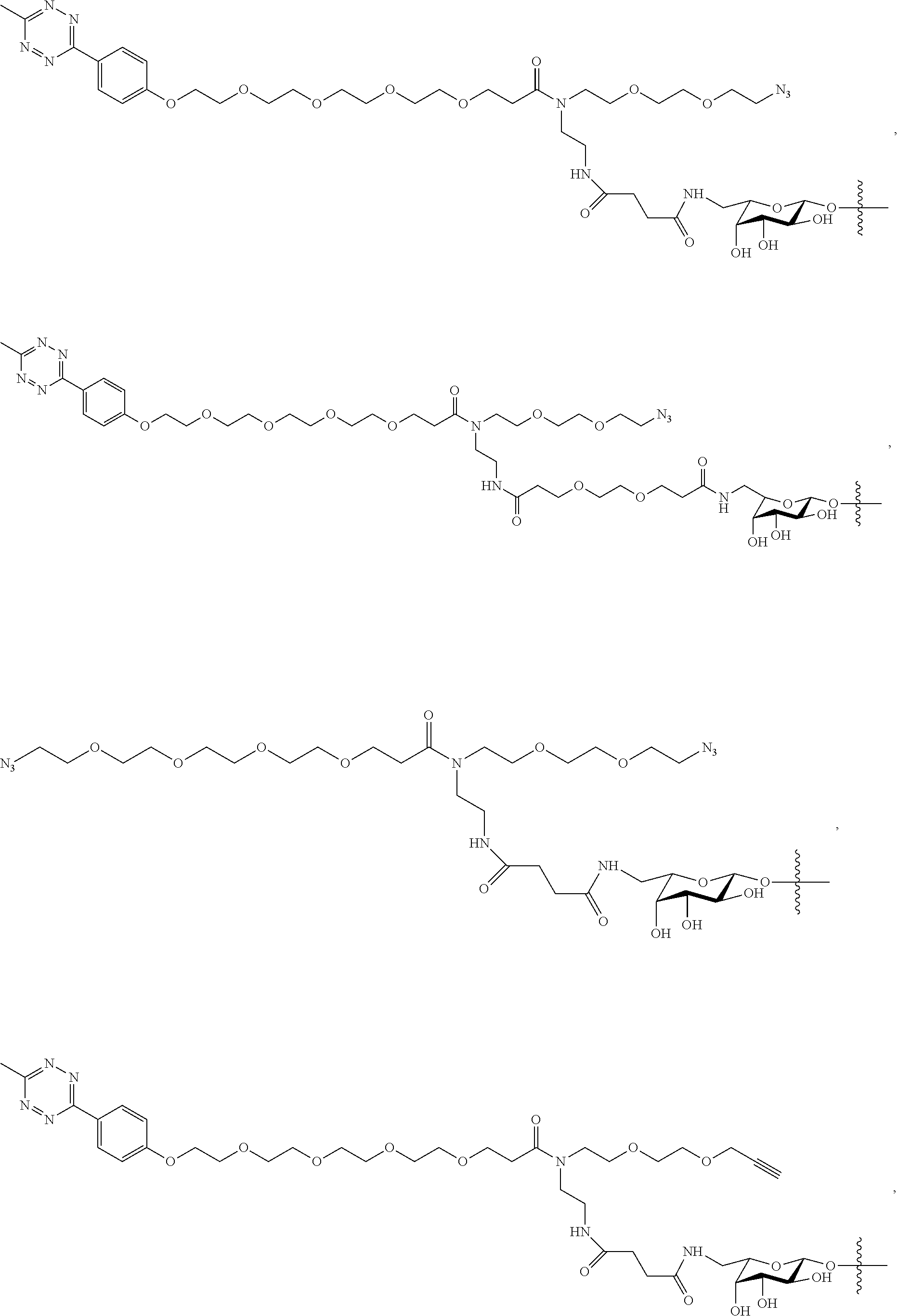

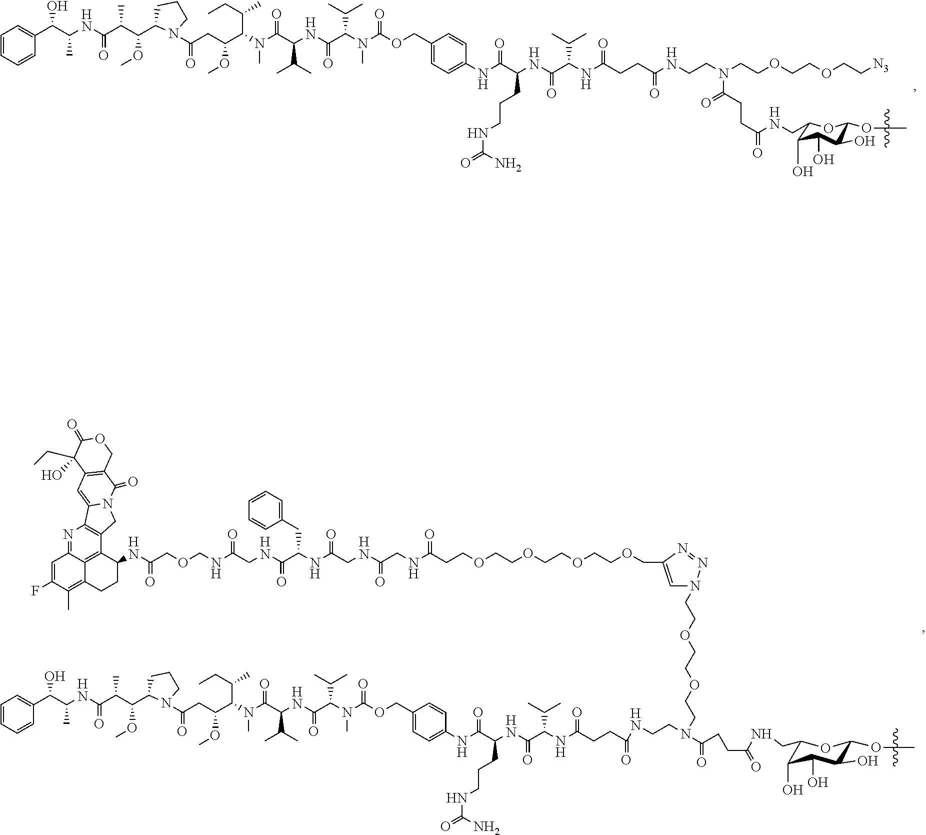

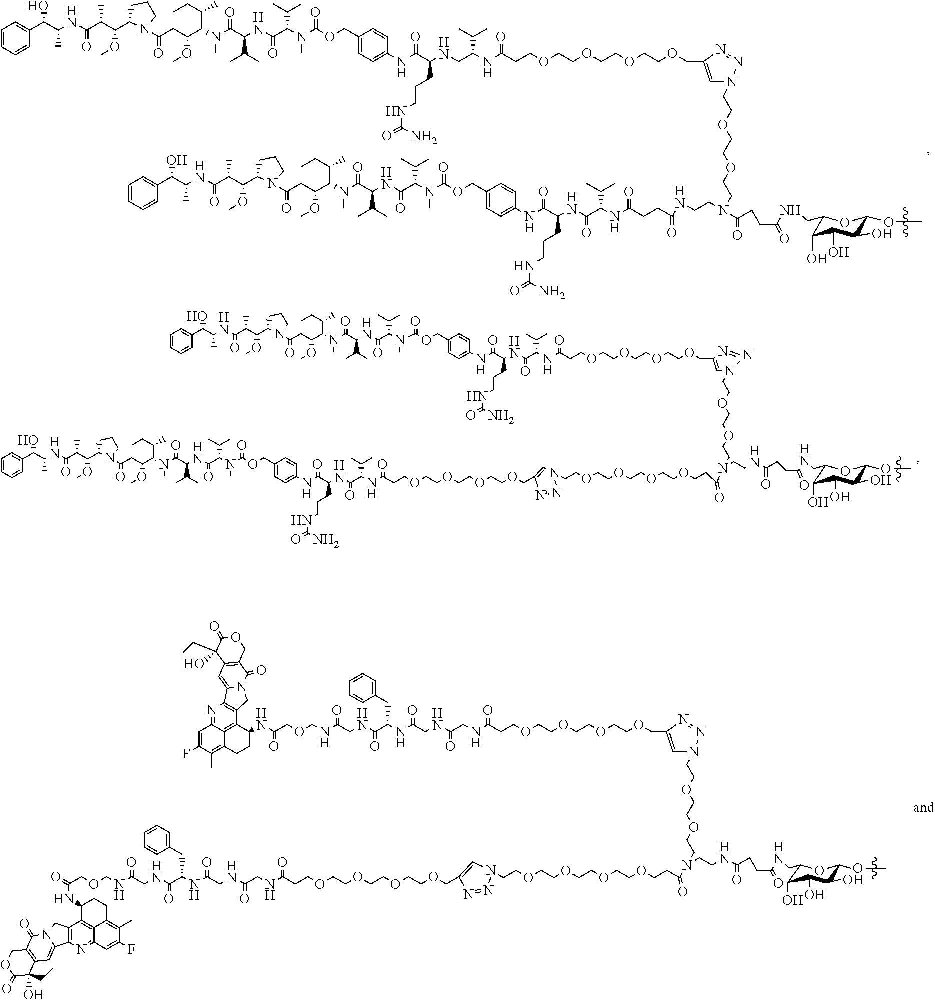

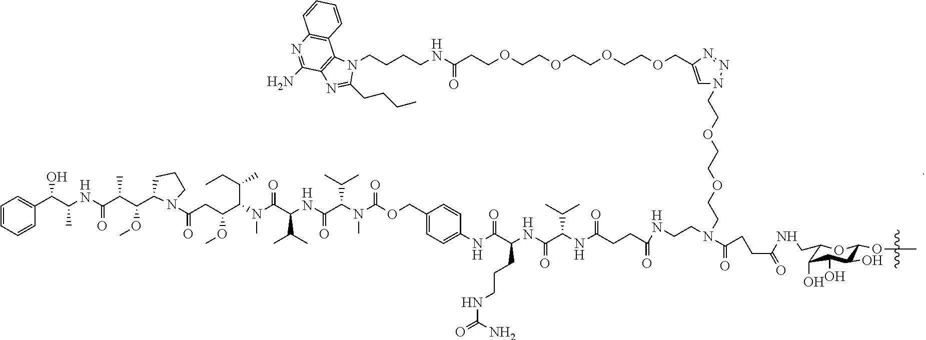

In some embodiments of the protein conjugate, in the protein conjugate of the present disclosure, said b is 0.

In some embodiments, the protein conjugate is obtained by reacting the protein conjugate of the present disclosure with one or more Y-(L′)_{c}-P_{F}′, wherein said Y comprises a functional moiety capable of reacting with said X_{F} and/or said X_{G}, L′ is a linker, e is 0 or 1, and said P_{F}′ is a biologically active molecule and/or a pharmaceutically active molecule.

In some embodiments, the protein conjugate has one or more of the following properties: have at least 2 MARs (active molecule to antibody ratio); have at least 2 MARs (active molecule to antibody ratio), and each MAR is about 2; have at least 2 MARs (active molecule to antibody ratio), and each MAR is about 4; capable of binding to an antigen; capable of binding to an antigen, with a similar binding affinity as the corresponding antibody; is stable in human plasma for at least 1 day; the linkage between the Fuc* and the GlcNAc of Formula (I) is stable in human plasma for at least 1 day; capable of participating in a bioorthogonal ligation reaction; and capable of inhibiting tumor growth and/or tumor cell proliferation.

In another aspect, the present disclosure provides a method for preparing the protein conjugate according to the present disclosure.

In another aspect, the present disclosure provides a method for preparing a protein conjugate, comprising step (a): contacting a fucose derivative donor Q-Fuc* with a protein comprising an oligosaccharide in the presence of a catalyst, wherein said oligosaccharide comprises Formula (VII): -GlcNAc(Fuc)_{b}-GalX (VII), to obtain a protein conjugate comprising the structure of Formula (I):

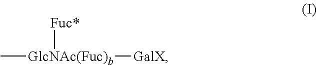

wherein: said GlcNAc is directly or indirectly linked to an amino acid of said protein; said GalX is an optionally substituted galactose; said Fuc is a fucose, and b is 0 or 1; said Q is a diphosphate ribonucleotide; and said Fuc* is a fucose derivative comprising two or more active molecules AM.

In some embodiments of the method, said Q is a uridine diphosphate (UDP), a guanosine diphosphate (GDP) or a cytidine diphosphate (CDP).

In some embodiments of the method, said Q-Fuc* is GDP-Fuc*.

In some embodiments of the method, said catalyst comprises a fucosyltransferase.

In some embodiments of the method, the fucosyltransferase is an α-1,3-fucosyltransferase or a functional variant or fragment thereof. In some embodiments, the fucosyltransferase is derived from bacteria. In some embodiments, the fucosyltransferase is derived from Helicobacter pylori. In some embodiments, the fucosyltransferase is derived from Helicobacter pylori 26695.

In some embodiments, said fucosyltransferase comprises an amino acid sequence as set forth in GenBank Accession No. AAD07710.1, or a functional variant and/or fragment thereof:

In some embodiments of the method, the fucosyltransferase comprises a catalytic region and one to ten HPR, said catalytic region comprises an amino acid sequence as set forth in SEQ ID NO: 13, and said HPR comprises an amino acid sequence as set forth in SEQ ID NO: 12.

In some embodiments of the method, the fucosyltransferase comprises a catalytic region and one to ten HPR, said catalytic region comprises an amino acid sequence as set forth in SEQ ID NO: 14, and said HPR comprises an amino acid sequence as set forth in SEQ ID NO: 12.

In some embodiments of the method, the fucosyltransferase comprises a catalytic region and one to ten HPR, said catalytic region comprises an amino acid sequence as set forth in SEQ ID NO: 15, and said HPR comprises an amino acid sequence as set forth in SEQ ID NO: 12.

In some embodiments of the method, the fucosyltransferase comprises an amino acid sequence as set forth in any of SEQ ID NO: 16, 18, 20, 22 and 24.

In some embodiments of the method, the catalyst further comprises a fusion tag.

In some embodiments of the method, the catalyst comprises an amino acid sequence as set forth in any of SEQ ID NO: 16-25.

In some embodiments of the method, the Fuc* comprises the structure of Formula (II):

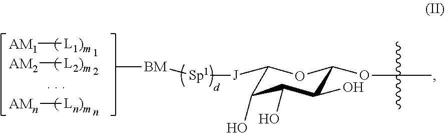

J is a jointer and is directly linked to the

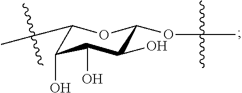

Sp¹ is a spacer moiety, d is 0 or 1; BM is a branching moiety; L₁ to Lₙ each independently is a linker, m₁ to mₙ each independently is 0 or 1; AM₁ to AMₙ each independently is an active molecule; and n is an integer from 2-10.

In some embodiments of the method, the J has a structure of:

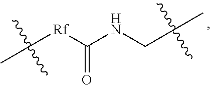

wherein R_{f} is —CH₂—, —NH— or —O—, and the right side of the structure is directly linked to the

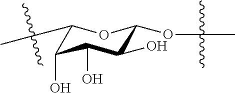

### of Formula (II).

In some embodiments of the method, the J is

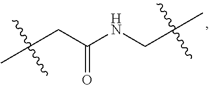 [SMILES: *CNC(=O)C*]

and the right side of the structure is directly linked to the

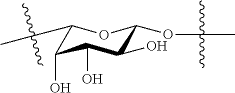

### of Formula (II).

In some embodiments of the method, the BM comprises

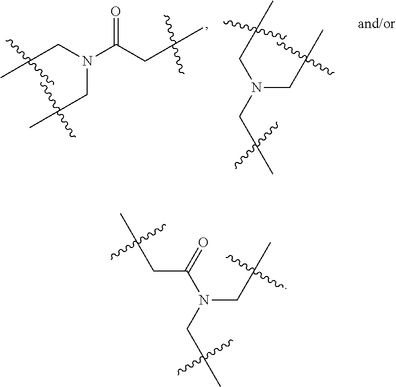 [SMILES: *CC(=O)N(C*)C* ; *CN(C*)C* ; *CC(=O)N(C*)C*]

In some embodiments of the method, n is 2, and said Fuc* comprises the structure of Formula (III):

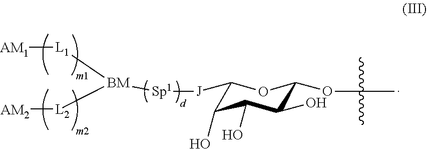

In some embodiments of the method, the BM is selected from the group consisting of:

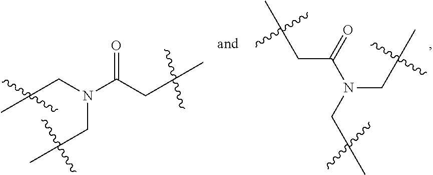 [SMILES: *CC(=O)N(C*)C* ; *CC(=O)N(C*)C*]

wherein the right side of the structure is directly linked to said Sp¹ or said J.

In some embodiments of the method, the Sp¹ is selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs², wherein each Rs¹ is independently selected from the group consisting of halogen, —OH, —NH₂ and —COOH, and each Rs² is independently selected from the group consisting of —O—, —S—,

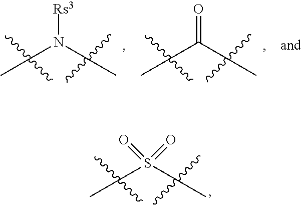

Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the method, the Sp¹ is selected from the group consisting of:

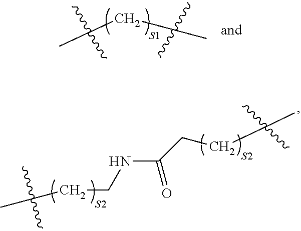

said S1 is independently an integer from 1-50, said S2 is independently an integer from 0-50, each said —CH₂—(—CH₂— in the parentheses) is independently optionally replaced by —O— with the proviso that two or more consecutive —CH₂— are not simultaneously replaced by —O—, the right side of the structure is linked to said J and the left side of the structure is linked to said BM.

In some embodiments of the method, each of L₁ to Lₙ is independently a linker of Formula (IV): (CL)_{y}-(FL)ₓ (IV), FL is a spacer moiety, x is 0 or 1; CL is a cleavable linker, y is 0 or 1; the right side of Formula (IV) is linked to said BM and the left side of Formula (IV) is linked to said AM.

In some embodiments of the method, the FL is a spacer moiety selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs², wherein each Rs¹ is independently selected from the group consisting of halogen, —OH, —NH₂ and —COOH, and each Rs² is independently selected from the group consisting of —O—, —S—,

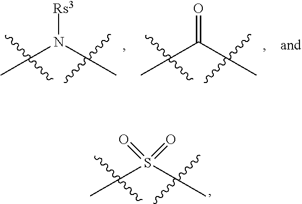

Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the method, the FL is a spacer moiety selected from the group consisting of:

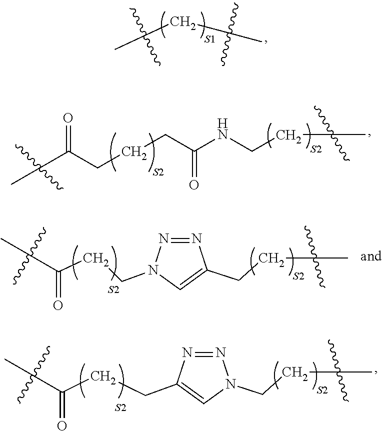

wherein said S1 is an integer from 1-50, each said S2 is independently an integer from 0-50, each said —CH₂—(—CH₂— in the parentheses) is independently optionally replaced by —O— with the proviso that two or more consecutive —CH₂— are not simultaneously replaced by —O—, the right side of the structure is linked to said BM, and the left side of the structure is linked to said CL or said AM.

In some embodiments of the method, the CL is an acid-labile linker, a redox-active linker, a photo-active linker and/or a proteolytically cleavable linker.

In some embodiments of the method, the CL is a vc-PAB-linker and/or a GGFG-linker.

In some embodiments of the method, each of AM₁ to AMₙ is independently a chemically active molecule, an enzymatically active molecule, a biologically active molecule, and/or a pharmaceutically active molecule.

In some embodiments of the method, the AM₁ to AMₙ independently comprises a chemically active molecule or enzymatically active molecule X_{F}.

In some embodiments of the method, the chemically or enzymatically active molecule X_{F} comprises a functional moiety capable of participating in a ligation reaction.

In some embodiments of the method, the X_{F} comprises a functional moiety capable of participating in a bioorthogonal ligation reaction.

In some embodiments of the method, the X_{F} comprises a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof:

In some embodiments of the method, the X_{F} comprises a functional moiety selected from the group consisting of:

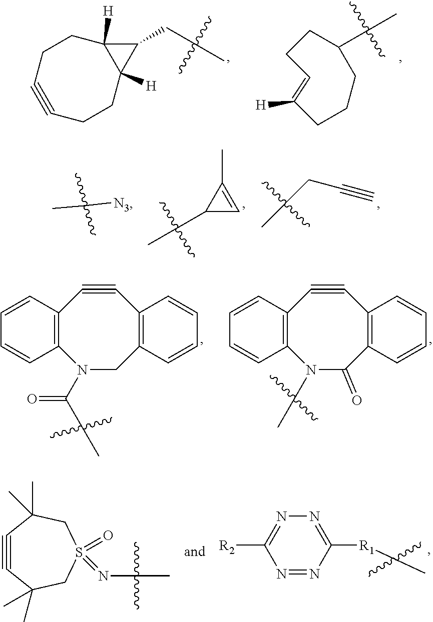

wherein R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group.

In some embodiments of the method, the X_{F} comprises a functional moiety selected from the group consisting of:

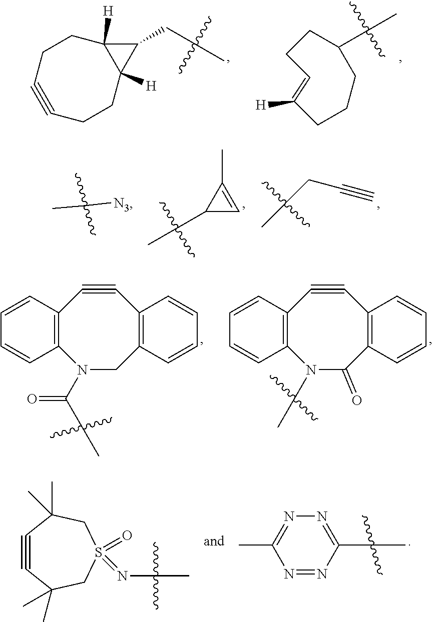

In some embodiments of the method, the AM₁ to AMₙ independently comprises a biologically active molecule and/or pharmaceutically active molecule P_{F}.

In some embodiments of the method, the P_{F} comprises a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof:

In some embodiments of the method, the P_{F} is a pharmaceutically active molecule.

In some embodiments of the method, the P_{F} comprises a cytotoxin, an agonist, an antagonist, an antiviral agent, an antibacterial agent, an oligonucleotide, a polypeptide or any combination thereof:

In some embodiments of the method, the P_{F} comprises a cytotoxin or an agonist.

In some embodiments of the method, the P_{F} comprises a DNA or RNA damaging agent, an RNA polymerase inhibitor, a topoisomerase inhibitor and/or a microtubule inhibitor.

In some embodiments of the method, the P_{F} comprises a pyrrolobenzodiazepine, auristatin, maytansinoids, duocarmycin, tubulysin, enediyene, doxorubicin, pyrrole-based kinesin spindle protein inhibitor, calicheamicin, amanitin, camptothecin and/or derivatives thereof:

In some embodiments of the method, the P_{F} comprises a MMAE, a DXd, T785 and/or derivatives thereof:

In some embodiments of the method, the GalX is linked to said GlcNAc through a β1,4 linkage.

In some embodiments of the method, the GalX is a galactose.

In some embodiments of the method, the GalX is a substituted galactose, and the hydroxyl group at one or more positions selected from the C2 position, the C3 position, the C4 position and the C6 position of the galactose, is substituted.

In some embodiments of the method, the GalX is a substituted galactose, wherein the hydroxyl group at the C2 position of the galactose is substituted.

In some embodiments of the method, the GalX is a monosaccharide.

In some embodiments of the method, the GalX is substituted by

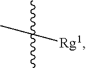

and said Rg¹ is selected from the group consisting of hydrogen, halogen, —NH₂, —SH, —N₃, —COOH, —CN, C₁-C₂₄ alkyl, C₃-C₂₄ cycloalkyl, C₂-C₂₄ alkenyl, C₅-C₂₄ cycloalkenyl, C₂-C₂₄ alkynyl, C₆-C₂₄ cycloalkynyl, C₂-C₂₄ (hetero)aryl, C₃-C₂₄ alkyl(hetero)aryl, C₃-C₂₄ (hetero)arylalkyl and any combination thereof, wherein each of said alkyl, cycloalkyl, alkenyl, cycloalkenyl, alkynyl, cycloalkynyl, (hetero)aryl, alkyl(hetero)aryl and (hetero)arylalkyl is independently optionally substituted by one or more Rs⁴ and/or is independently optionally interrupted by one or more Rs⁵, wherein each Rs⁴ is independently selected from the group consisting of halogen, —OH, —NH₂, —SH, —N₃, —COOH and —CN, and each Rs⁵ is independently selected from the group consisting of —O—, —S—,

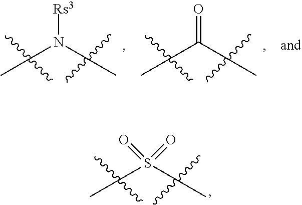

and Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the method, the GalX is substituted by

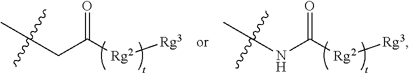

wherein t is 0 or 1, Rg² is selected from the group consisting of C₁-C₂₄ alkylene, C₃-C₂₄ cycloalkylene, C₂-C₂₄ alkenylene, C₅-C₂₄ cycloalkenylene, C₂-C₂₄ alkynylene, C₆-C₂₄ cycloalkynylene, C₂-C₂₄ (hetero)arylene, C₃-C₂₄ alkyl(hetero)arylene and C₃-C₂₄ (hetero)arylalkylene, wherein each of said alkylene, cycloalkylene, alkenylene, cycloalkenylene, alkynylene, cycloalkynylene, (hetero)arylene, alkyl(hetero)arylene and (hetero)arylalkylene is independently optionally substituted by one or more Rs⁴ and/or is independently optionally interrupted by one or more Rs⁵, Rg³ is selected from the group consisting of hydrogen, halogen, —OH, —NH₂, —SH, —N₃, —COOH, —CN, C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl, C₂-C₂₄ (hetero)aryl and any combination thereof, wherein each of said C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl and C₂-C₂₄(hetero)aryl is independently optionally substituted by one or more Rs⁴, each Rs⁴ is independently selected from the group consisting of halogen, —OH, —NH₂, —SH, —N₃, —COOH and —CN, and each Rs^{s} is independently selected from the group consisting of —O—, —S—,

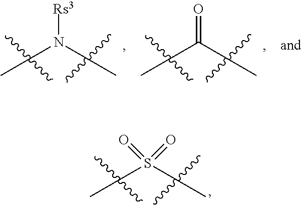

wherein Rs³ is selected from the group consisting of hydrogen, C₁-C₂₄ alkyl, C₂-C₂₄ alkenyl, C₂-C₂₄ alkynyl and C₃-C₂₄ cycloalkyl.

In some embodiments of the method, the GalX comprises a chemically active molecule and/or enzymatically active molecule X_{G}.

In some embodiments of the method, the X_{G} comprises a functional moiety capable of participating in a ligation reaction.

In some embodiments of the method, the X_{G} comprises a functional moiety capable of participating in a bioorthogonal ligation reaction.

In some embodiments of the method, X_{G} comprises a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof:

In some embodiments of the method, the X_{G} comprises a

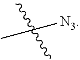 [SMILES: *N=[N+]=[N-]]

In some embodiments of the method, the GalX is selected from the group consisting of:

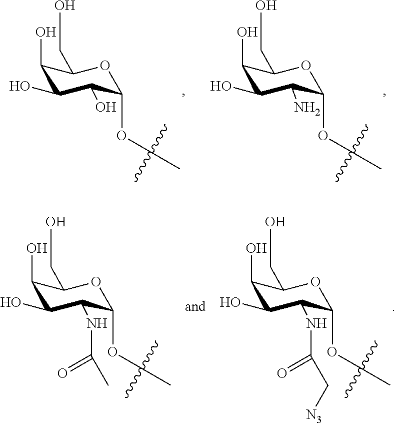

In some embodiments of the method, the protein comprises an antigen binding fragment and/or an Fc fragment.

In some embodiments of the method, the oligosaccharide is an N-linked oligosaccharide.

In some embodiments of the method, the oligosaccharide is linked to an Asparagine (Asn) residue of said protein.

In some embodiments of the method, the GlcNAc of Formula (VII) is directly linked to an Asn residue of said protein.

In some embodiments of the method, the GlcNAc of Formula (VII) is linked to a saccharide of said oligosaccharide.

In some embodiments of the method, the GlcNAc of Formula (VII) is linked to a mannose of said oligosaccharide, and optionally b is 0.

In some embodiments of the method, the protein comprises a Fc fragment, and said oligosaccharide is linked to said Fc fragment.

In some embodiments of the method, the protein comprises a Fc fragment, and said oligosaccharide is linked to the CH2 domain of said Fc fragment.

In some embodiments of the method, the protein comprises a Fc fragment, and said oligosaccharide is linked to the Asn297 of said Fc fragment, numbered according to the Kabat numbering system.

In some embodiments of the method, the protein is an antibody. In some embodiments, the protein is a monoclonal antibody. In some embodiments, the protein is an IgG antibody. In some embodiments, the protein is a humanized antibody.

In some embodiments of the method, said Fuc* is linked to said GlcNAc through an α1,3 linkage.

In some embodiments of the method, b is 1, and said Fuc is linked to said GlcNAc through an a1,6 linkage.

In some embodiments of the method, n is 2, said Fuc* comprises the AM₁ and the AM₂, both said AM₁ and said AM₂ comprises a X_{F}, the X_{F} of AM₁ and the X_{F} of AM₂ are identical or different.

In some embodiments of the method, n is 2, said Fuc* comprises the AM₁ and the AM₂, both said AM₁ and said AM₂ comprises a X_{F}, the X_{F} of AM₁ and the X_{F} of AM₂ is independently selected from the group consisting of:

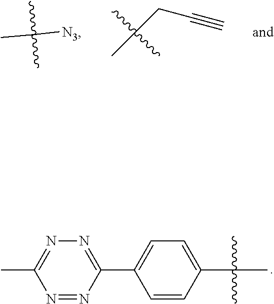

In some embodiments of the method, the X_{F} of AM₁ does not react bioorthogonally with the X_{F} of AM₂.

In some embodiments of the method, n is 2, said Fuc* comprises the AM₁ and the AM₂, the AM₁ comprises a X_{F} and the AM₂ comprises a P_{F}, or the AM₁ comprises a P_{F} and the AM₂ comprises a X_{F}.

In some embodiments of the method, n is 2, said Fuc* comprises the AM₁ and the AM₂, both said AM₁ and said AM₂ comprises a P_{F}, the P_{F} of AM₁ and the P_{F} of AM₂ are identical or different.

In some embodiments of the method, Q-Fuc* is of a structure selected from the followings:

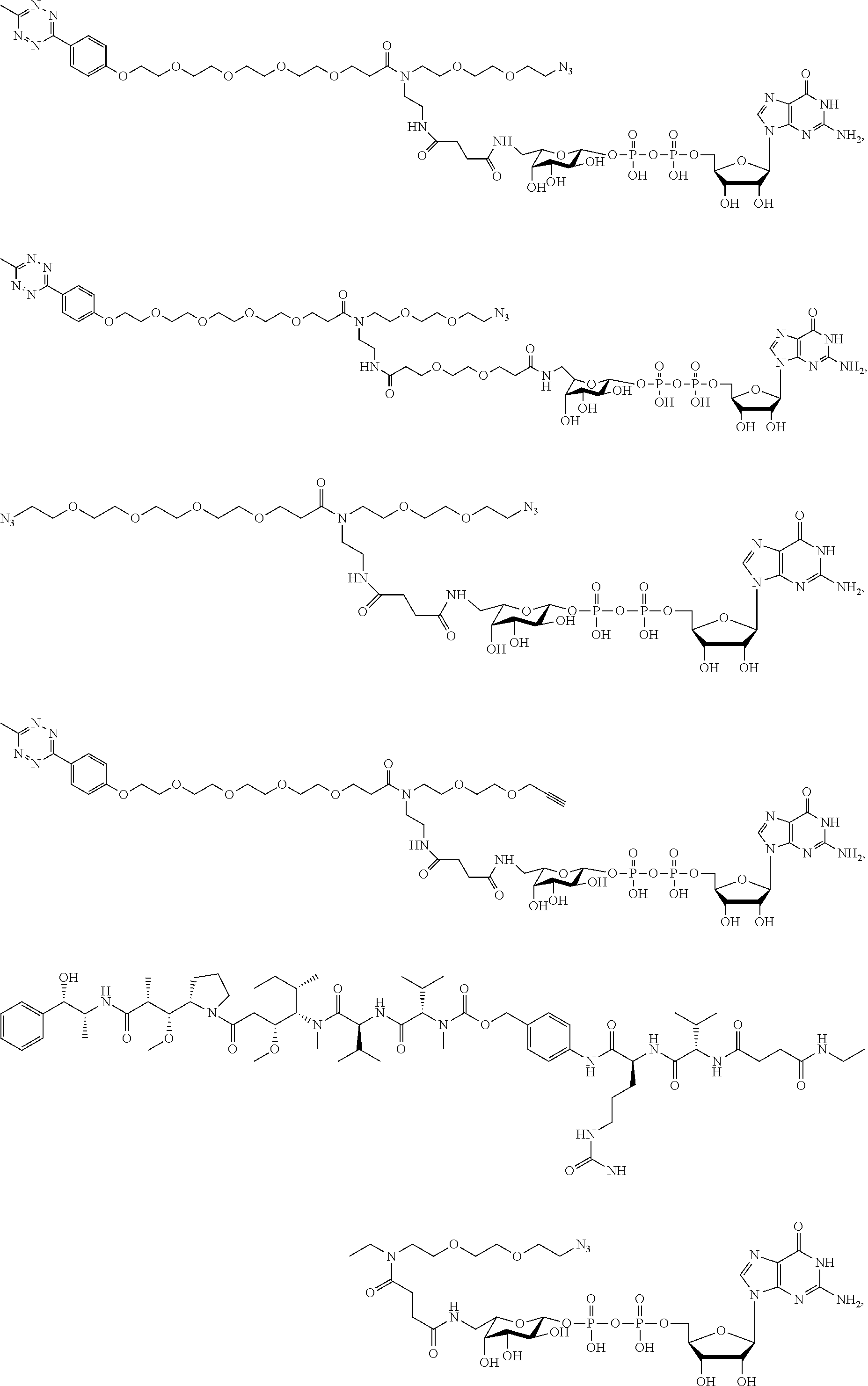

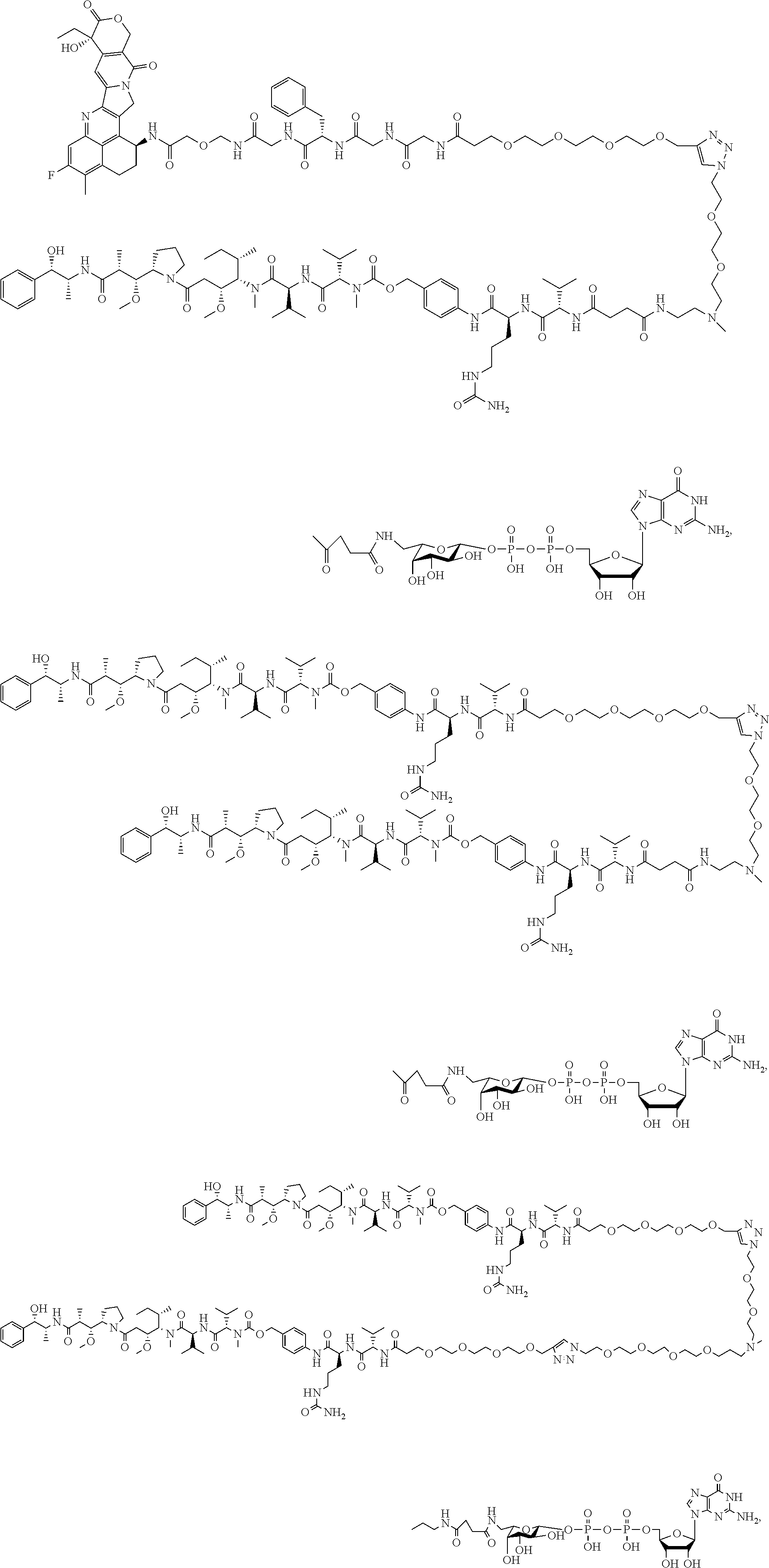

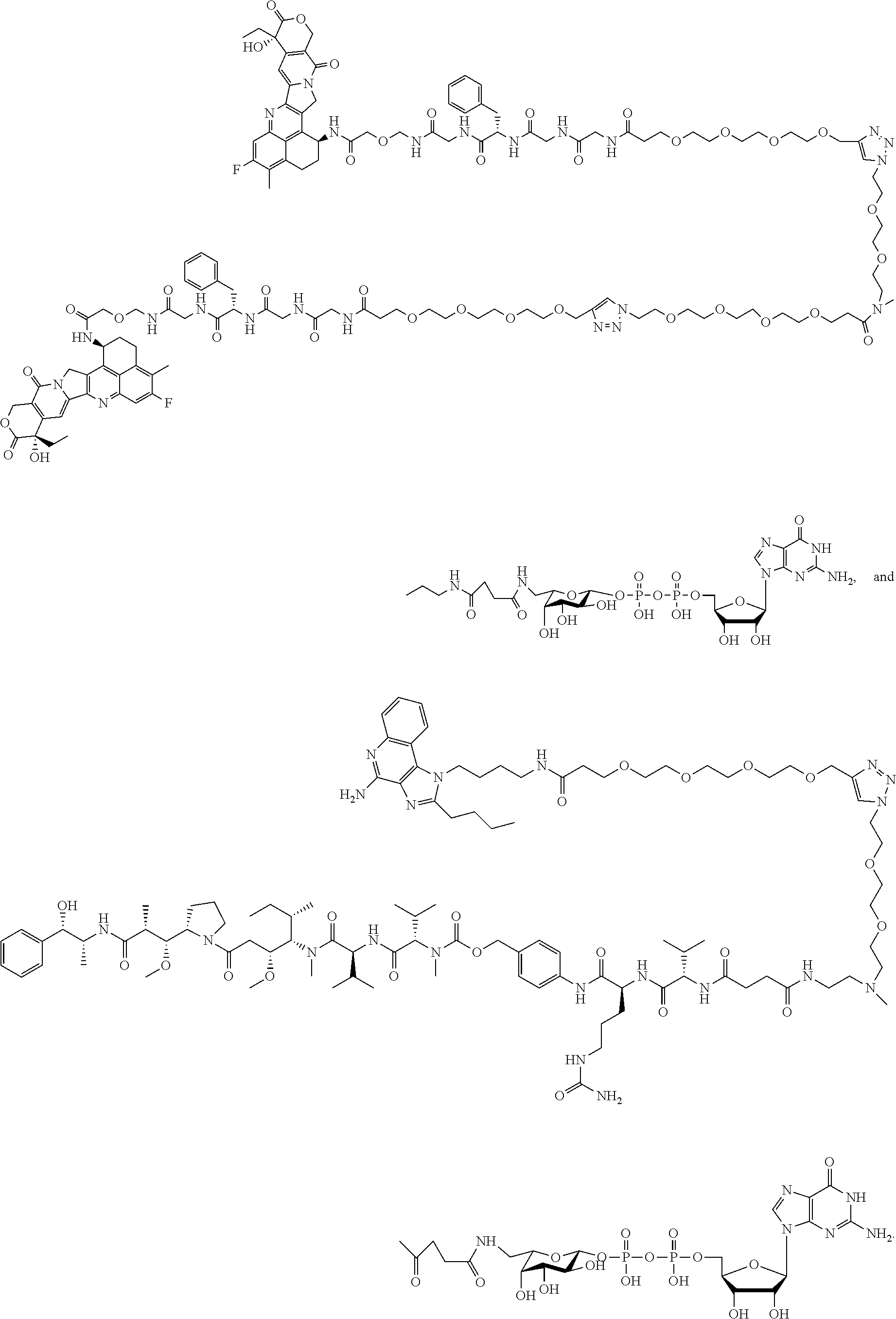

In some embodiments of the method, the protein comprises 1-20 of said structure of -GlcNAc(Fuc)_{b}-GalX (VII).

In some embodiments of the method, the protein comprises 2 or 4 of said structure of -GlcNAc(Fuc)_{b}-GalX (VII).

In some embodiments of the method, the protein comprises 2 of said structure of -GlcNAc(Fuc)_{b}-GalX (VII).

In some embodiments of the method, the protein comprising the oligosaccharide comprises a structure of Formula (VIII)

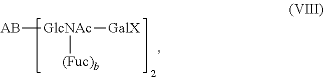

wherein said AB is an antibody comprising a Fc fragment or a Fc-fusion protein, the GlcNAc is directly linked to an Asn of the Fc fragment of the AB, the Fuc is linked to the GlcNAc through an α1,6 linkage, the GalX is linked to the GlcNAc through a β1,4 linkage, and b is 0 or 1.

In some embodiments, the method further comprises the steps of: i) modifying a glycosylated antibody comprising the Fc fragment or the Fc-fusion protein with an endoglycosidase to obtain a modified protein; and ii) contacting the modified protein with a UDP-GalX in the presence of a catalyst to obtain said protein comprising the structure of Formula (VIII); said b is 0 or 1.

In some embodiments, the method further comprises the steps of: i) modifying a glycosylated antibody comprising an Fc fragment or the Fc-fusion protein with an endoglycosidase and an α1,6 fucosidase to obtain a modified protein; and ii) contacting the modified protein with a UDP-GalX in the presence of a catalyst to obtain said protein comprising the structure of Formula (VIII); said b is 0.

In some embodiments of the method, the protein comprises 4 of said structure of -GlcNAc(Fuc)_{b}-GalX (VII).

In some embodiments of the method, the protein comprises the structure of Formula (IX):

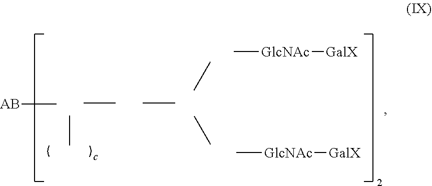

wherein said AB is an antibody comprising a Fc fragment or a Fc-fusion protein, [symbol] is a GlcNAc, [symbol] is a mannose, [symbol] is a fucose linked to the [symbol] through a α1,6 linkage, c is 0 or 1; said oligosaccharide is linked to an Asn of the Fc fragment of the AB through the [symbol] , and the GalX is linked to the GlcNAc through a β1,4 linkage.

In some embodiments, the method further comprises contacting an antibody comprising an Fc fragment or the Fc-fusion protein having a glycoform of G₀(F)_{0,1}, G₁(F)_{0,1} and/or G₂(F)_{0,1} with a UDP-GalX in the presence of a catalyst, to obtain said protein comprising the structure of Formula (IX).

In some embodiments, the method further comprises contacting an antibody comprising an Fc fragment or the Fc-fusion protein having a glycoform of G₀(F)_{0,1} with a UDP-GalX in the presence of a catalyst, to obtain said protein comprising the structure of Formula (IX).

In some embodiments of the method, b is 0.

In some embodiments, the method comprises contacting the protein conjugate of the present disclosure with one or more Y-(L′)e-P_{F}′, wherein said Y comprises a functional moiety capable of reacting with said X_{F} and/or said X_{G}, L′ is a linker, e is 0 or 1, and said P_{F}′ is a biologically active molecule and/or a pharmaceutically active molecule.

In another aspect, the present disclosure provides a use of the Q-Fuc* according to the present disclosure in preparation of a protein conjugate.

In another aspect, the present disclosure provides a protein conjugate, obtained with the method of the present disclosure.

In another aspect, the present disclosure provides a composition, comprising the protein conjugate of the present disclosure.

In some embodiments of the composition, the protein conjugates therein have at least 2 average MARs, and each of the average MARs is 1.6-2.0.

In some embodiments of the composition, the protein conjugates therein have at least 2 average MARs, and each of the average MARs is 3.2-4.0.

In some embodiments, the composition comprises a pharmaceutical composition.

In some embodiments, the composition further comprises a pharmaceutically acceptable carrier.

In another aspect, the present disclosure provides a method for preventing or treating a disease, comprising administrating the protein conjugate and/or the composition of the present disclosure.

In another aspect, the present disclosure provides use of the protein conjugate or the composition of the present disclosure in the preparation of a medicament for preventing or treating a disease.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWING

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “FIG.”, “Fig.” and “FIG.” herein), of which:

FIG. 1 illustrates an exemplary preparation method of the protein conjugate of the present disclosure. [symbol] is a GlcNAc, [symbol] is an α1,6 fucose, ⊗ is a galactose or substituted galactose, [symbol] is a protein comprising a Fc domain (e.g. antibody, Fc-fusion protein). Fuc* is according to the formula

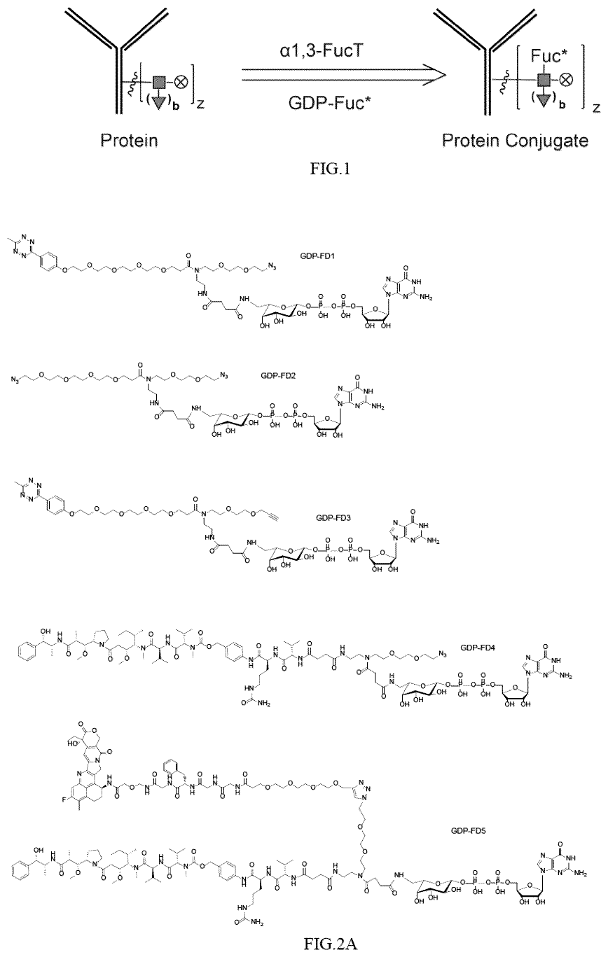

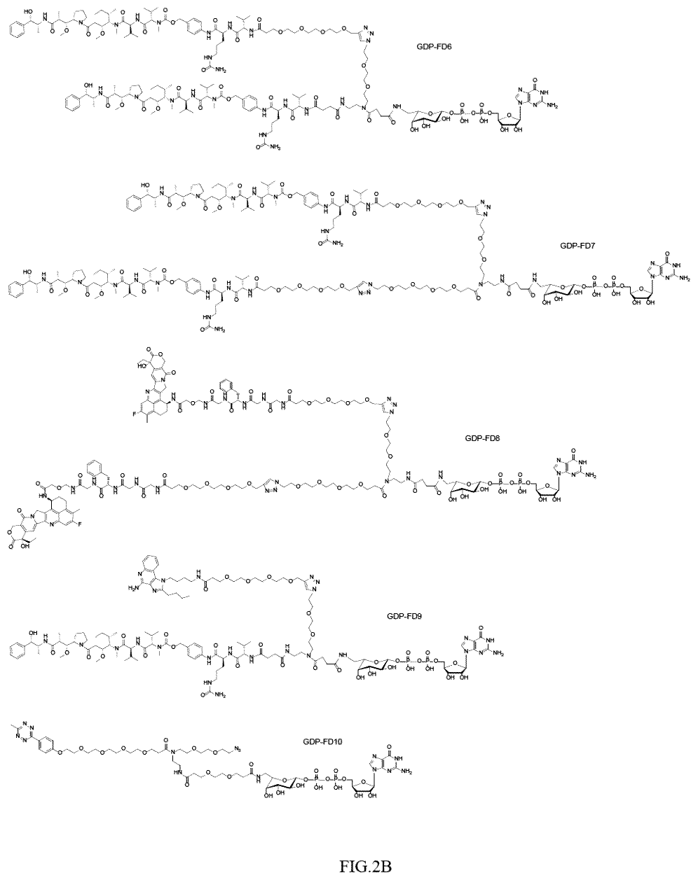

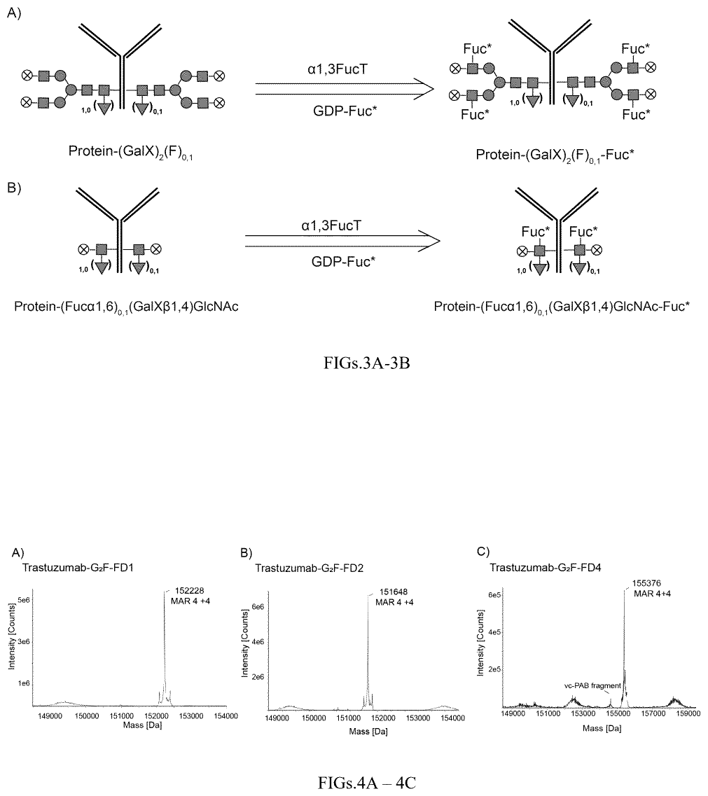

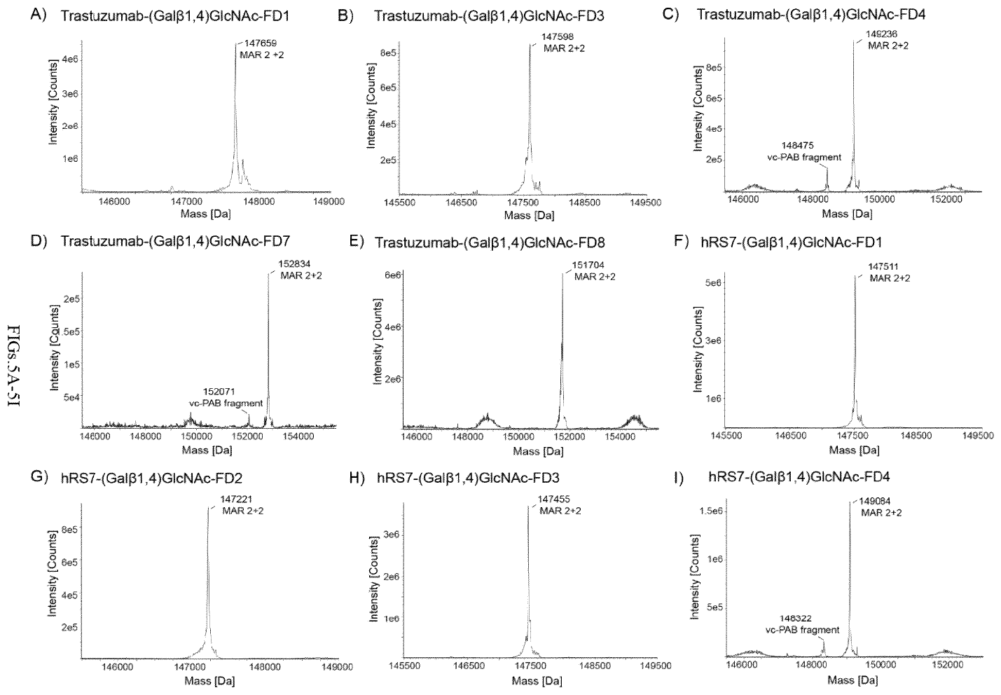

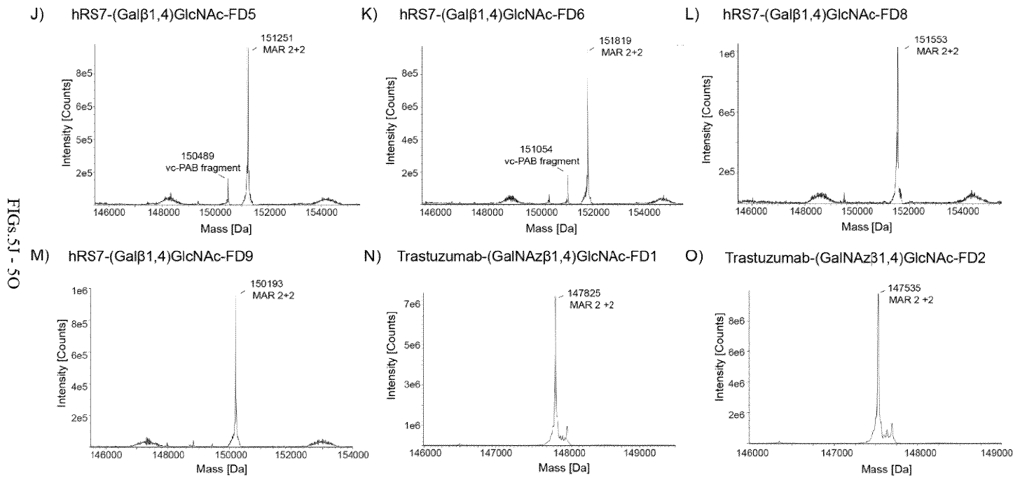

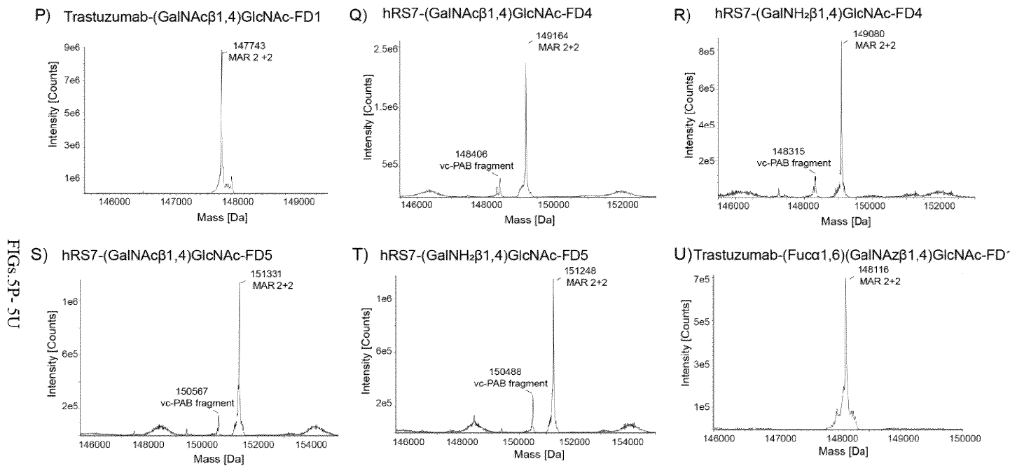

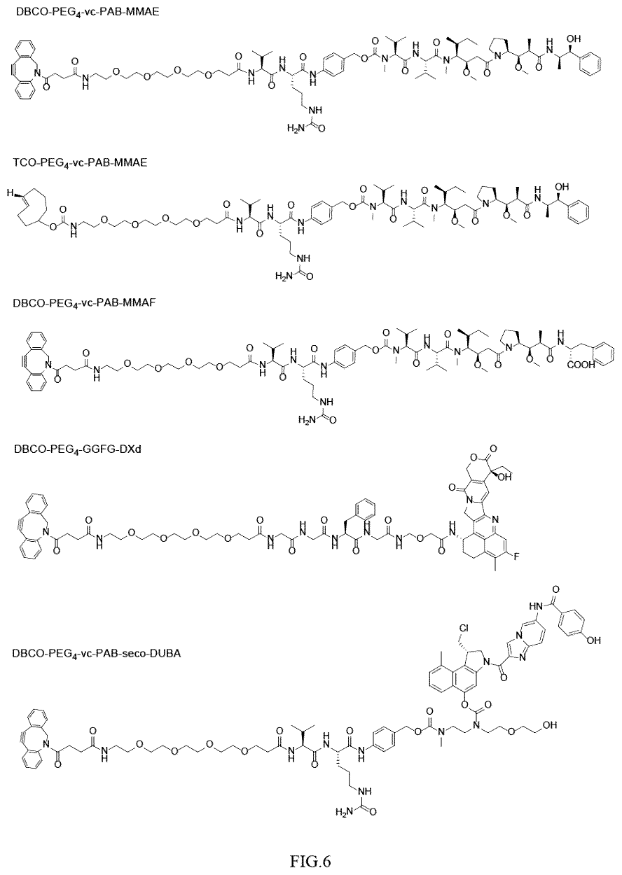

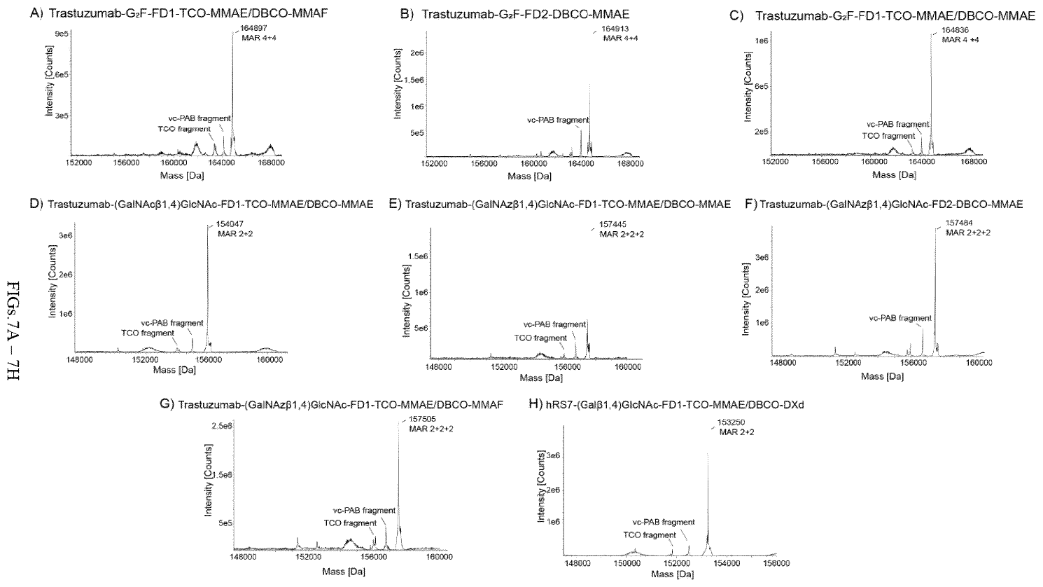

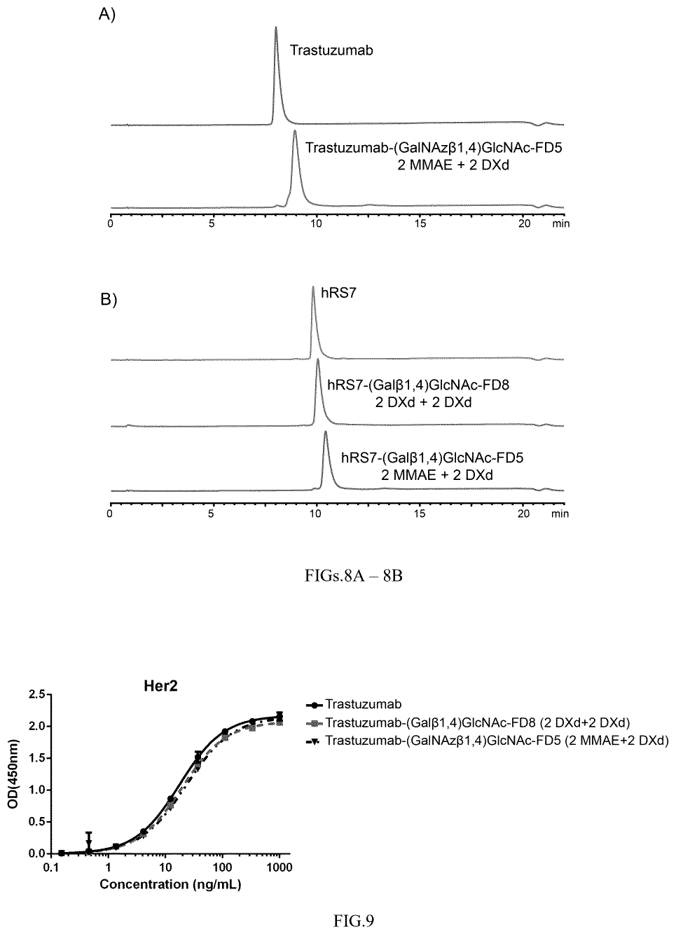

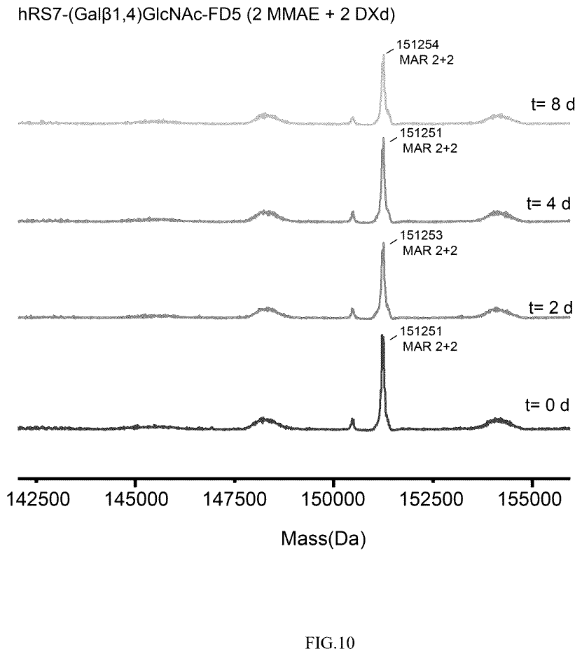

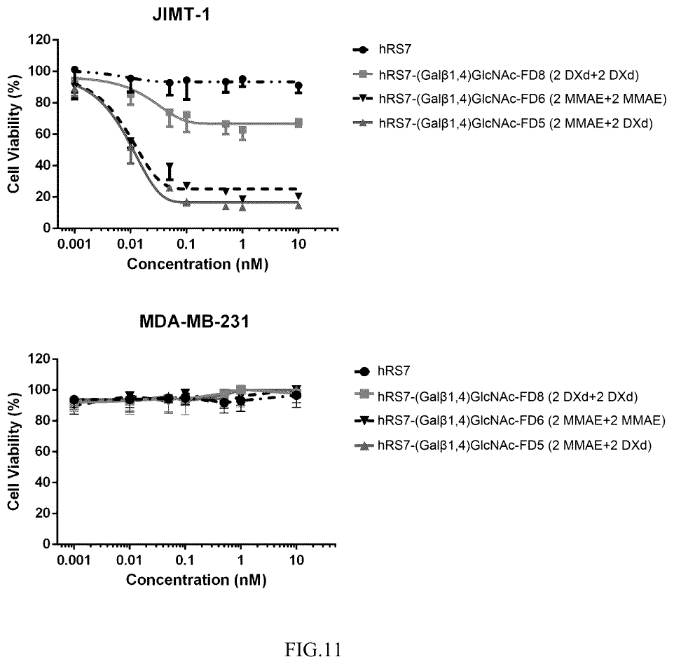

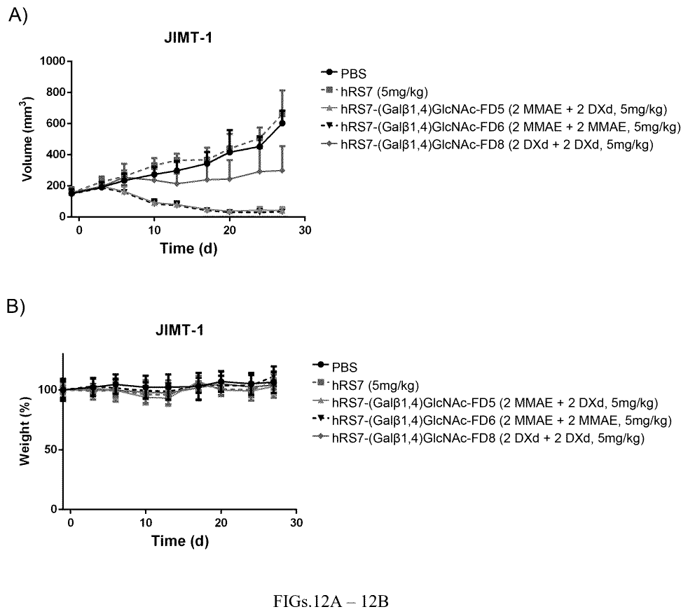

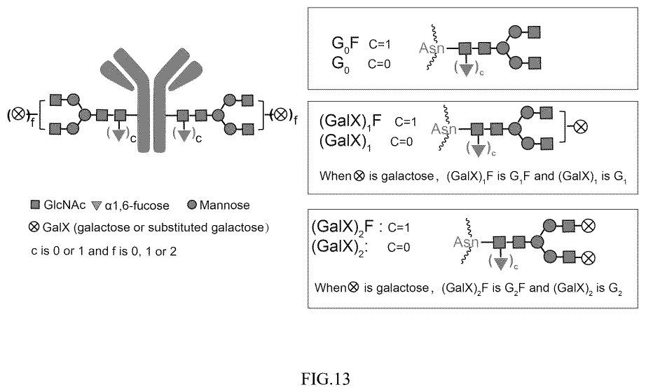

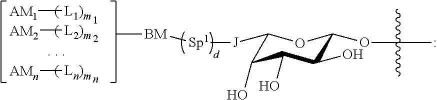

wherein J is a jointer and is directly linked to the

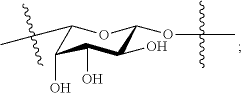

Sp¹ is a spacer moiety, d is 0 or 1; BM is a branching moiety; L₁ to Lₙ each independently is a linker, m₁ to mₙ each independently is 0 or 1; AM₁ to AMₙ each independently is an active molecule; and n is an integer from 2-10.

FIGS. 2A-2B illustrate the molecular structure of exemplary Q-Fuc* of the present disclosure.

FIGS. 3A-3B illustrate exemplary preparation method of the protein conjugates of the present disclosure. [symbol] is a GlcNAc, [symbol] is an α1,6 fucose, ⊗ is a galactose or substituted galactose, [symbol] is a protein comprising a Fc domain (e.g. antibody, Fc-fusion protein). Fuc* is according to the formula

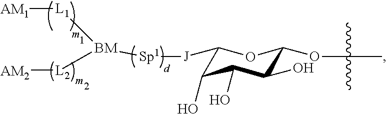

J is a jointer and is directly linked to the

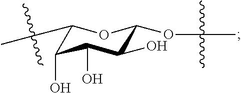

Sp¹ is a spacer moiety, d is 0 or 1; BM is a branching moiety; L₁ and L₂ each independently is a linker, m₁ and m₂ each independently is 0 or 1; AM₁ and AM₂ each independently is an active molecule.

FIGS. 4A-4C illustrate the MS analysis results of exemplary protein conjugates of the present disclosure.

FIGS. 5A-5U illustrate the MS analysis results of exemplary protein conjugates of the present disclosure.

FIG. 6 illustrates the molecular structure of exemplary Y-(L′)ₑ-P_{F} of the present disclosure.

FIGS. 7A-7H illustrate the MS analysis results of exemplary protein conjugates of the present disclosure.

FIGS. 8A-8B illustrate the HIC-HPLC analysis results of exemplary protein conjugates of the present disclosure.

FIG. 9 illustrates the binding (ELISA analysis) of exemplary protein conjugates of the present disclosure to their antigen.

FIG. 10 illustrates the stability of exemplary protein conjugates of the present disclosure in human plasma.

FIG. 11 illustrates the in vitro cytotoxicity of exemplary protein conjugates of the present disclosure.

FIGS. 12A-12B illustrate the in vivo efficacy of exemplary protein conjugates of the present disclosure.

FIG. 13 illustrates the glycoforms of a protein (e.g., an antibody comprising a Fc fragment or a Fc-fusion protein) of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “conjugate”, as used herein, generally refers to any substance formed from the joining together of separate parts. In the conjugate, the separate parts may be joined at one or more active site with each other. For example, the separate parts may be covalently or non-covalently associated with, or linked to, each other and exhibit various stoichiometric molar ratios. The conjugate may comprise peptides, polypeptides, proteins, drugs, prodrugs, polymers, nucleic acid molecules, small molecules, binding agents, mimetic agents, synthetic drugs, inorganic molecules, organic molecules and radioisotopes.

The term “Fc fragment” or “Fc domain”, as used herein, generally refers to a portion of an antibody constant region. For example, the term Fc fragment may refer to a protease (e.g., papain) cleavage product encompassing the paired CH2, CH3 and hinge regions of an antibody. In the context of this disclosure, the term Fc fragment or Fc refers to any polypeptide (or nucleic acid encoding such a polypeptide), regardless of the means of production, that includes all or a portion of the CH2, CH3 or hinge region of an immunoglobulin polypeptide.

The term “antigen binding fragment”, as used herein, generally refers to a peptide fragment capable of binding antigen. The antigen binding fragment may be a fragment of an immunoglobulin molecule. An antigen-binding fragment may comprise one light chain and part of a heavy chain with a single antigen-binding site. An antigen-binding fragment may be obtained by papain digestion of an immunoglobulin molecule. For example, an antigen-binding fragment may be composed of one constant and one variable domain of each of the heavy and the light chain. The variable domain may contain the paratope (the antigen-binding site), comprising a set of the complementarity determining regions, at the amino-terminal end of the immunoglobulin molecule. For example, the antigen binding fragment may be a Fab, a F(ab)₂, F(ab′), a F(ab′)₂, a ScFv, and/or a nanobody.

The term “antibody”, as used herein, generally refers to a polypeptide or a protein complex that specifically binds an epitope of an antigen or mimotope thereof: An antibody includes an intact antibody, or a binding fragment thereof that competes with the intact antibody for specific binding and includes chimeric, humanized, fully human, and bispecific antibodies. Binding fragments include, but are not limited to, Fab, Fab′, F(ab′)₂, Fv, single-chain antibodies, nanobodies and disulfide-linked Fvs (sdFv) fragments. In some embodiments, an antibody is referred to as an immunoglobulin and include the various classes and isotypes, such as IgA (IgA1 and IgA2), IgD, IgE, IgM, and IgG (IgG1, IgG3 and IgG4) etc. In some embodiments the term “antibody” as used herein refers to polyclonal and monoclonal antibodies and functional fragments thereof. An antibody includes modified or derivatized antibody variants that retain the ability to specifically bind an epitope. Antibodies are capable of selectively binding to a target antigen or epitope. In some embodiments, the antibody is from any origin, such as mouse or human, including a chimeric antibody thereof. In some embodiments, the antibody is humanized.

The term “monoclonal antibody”, as used herein, generally refers to an antibody obtained from a population of substantially homogeneous antibodies, i.e., the individual antibodies within the population are identical except for possible naturally occurring mutations and/or post-translational modifications (e.g., isomerizations, amidations) that may be present in minor amounts.

The term “IgG”, as used herein, generally refers to immunoglobulin G. Those skilled in the art will appreciate that immunoglobulin heavy chains are classified as gamma, mu, alpha, delta, or epsilon, (γ, μ, α, δ, ε) with some subclasses among them (e.g., γ₁-γ₄ or α₁-α₂). It is the nature of this chain that determines the “isotype” of the antibody as IgG, IgM, IgA IgG, or IgE, respectively. The immunoglobulin subclasses (subtypes) e.g., IgG1, IgG2, IgG3, IgG4, IgA1, IgA2, etc. are well characterized and are known to confer functional specialization. Human IgG is typically characterized by glycosylation at position Asn297 (numbering according to Kabat numbering system) in the heavy chain CH2 region of the Fc region.

In the present disclosure, “Asn297” and “N297” can be used interchangeably, and generally refers to the Asparagine at site 297 (numbered according to the Kabat numbering system, (Kabat et al., Sequences of Proteins of Immunological Interest, Vol. 1, 5th Ed. U.S. Public Health Service, National Institutes of Health. NIH Publication No. 91-3242; Copyright 1991)) of an antibody Fc fragment. Asn297 of an antibody or antibody fragment may be attached with one or more oligosaccharide.

The term “humanized antibody”, as used herein, generally refers to an antibody with some or all CDRs from a non-human species, while the framework region and constant region thereof contain amino acid residues derived from a human antibody.

The term “Fc-fusion protein”, as used herein, generally refers to a protein which are composed of the Fc fragment of an immunoglobulin genetically linked to a peptide or protein of interest. In some embodiment, the protein conjugate of the present disclosure is a Fc-fusion protein conjugate.

In the present disclosure, the term “GlcNAc” and “N-acetylglucosamine” can be used interchangeably, and generally refers to an amide derivative of the monosaccharide glucose.

Glycosylation generally refers to a reaction wherein a carbohydrate, i.e., a glycosyl donor, is attached to a hydroxyl or other functional group of another molecule (a glycosyl acceptor). In some embodiments, glycosylation mainly refers to the enzymatic process that attaches glycans to proteins, or other organic molecules. The glycosylation in protein can be modified in glycosylation linkage, glycosylation structure, glycosylation composition and/or glycosylation length. Glycosylation can comprise N-linked glycosylation, O-linked glycosylation, phosphoserine glycosylation, C-mannosylation, formation of GPI anchors (glypiation), and/or chemical glycosylation. Correspondingly, a glycosylated oligosaccharide of a protein can be a N-linked oligosaccharide, O-linked oligosaccharide, phosphoserine oligosaccharide, C-mannosylated oligosaccharide, glypiated oligosaccharide, and/or chemical oligosaccharide.

The term “N-linked oligosaccharide”, as used herein, generally refers to the attachment of an oligosaccharide to a nitrogen atom. In some embodiments, the oligosaccharide comprises a carbohydrate consisting of several sugar molecules, sometimes also referred to as glycan. In some embodiments, the nitrogen atom is an amide nitrogen of an amino acid residue of a protein, for example, an asparagine (Asn) of a protein.

The term “directly linked” as used herein, generally refers to that a moiety is linked to another moiety without any intermediate moiety or linker. For example, a GlcNAc directly linked to an amino acid residue of an antibody generally refers to that the GlcNAc is attached via a covalent bond to an amino acid residue of the antibody, for example, via an N-glycosidic bond to an amide nitrogen atom in a side chain of an amino acid (e.g., an asparagine amino acid) of the antibody. In the present disclosure, when a GlcNAc is “indirectly linked” to an amino acid of the protein, there are usually at least one monosaccharide moiety between the GlcNAc and the amino acid of the protein.

The term “active molecule” or AM, as used herein, generally refers to a molecule with a desired characteristic. The desired characteristic may be a physical characteristic or a chemical characteristic, for example, reactive activity, stability, solubility, binding activity, inhibiting activity, toxicity or degradability. An AM may comprise any substances possessing a desired biological activity and/or a reactive functional moiety that may be used to incorporate a drug into the protein conjugate of the disclosure. For example, an AM may be a chemically active molecule, an enzymatically active molecule, a biologically active molecule, and/or a pharmaceutically active molecule. For example, the active molecule may be a therapeutical agent, a diagnosis agent, a pharmacological agent and/or a biological agent, e.g., a cytotoxin, a cytostatic agent, a radioisotope or radionuclide, a metal chelator, an oligonucleotide, an antibiotic, a fluorophore, a biotin tag, a polypeptide, or any combination thereof: In some cases, an active molecule could be a chemically active molecule. For example, a chemically active molecule may be a chemically functional moiety that could react with another chemically functional moiety to form a covalent bond. For example, a chemically active molecule may be able to participate in a ligation reaction. In some cases, an active molecule could be an enzymatically active molecule that could react with a correspondingly complementary functional moiety to form a covalent bond in the presence of an enzyme.

The term “functional moiety” as used herein, generally refers to a group capable of reacting with another group. A functional moiety can be used to incorporate an agent (e.g., an agent without a reactive activity or with a low reactive activity) into a protein or a protein conjugate. For example, the agent may be a pharmaceutically active molecule (e.g. a cytotoxin). A functional moiety may be a chemical group or a residue having chemical and/or enzymatic reactivity. In some embodiments, a functional moiety may be a group capable of reacting in a ligation reaction.

The term “ligation reaction” as used herein, generally refers to a chemical and/or an enzymatic reaction in which a molecule is capable of being linked to another molecule. This binding may be driven by the functional moiety of the reacting molecules.

The term “bioorthogonal ligation reaction” as used herein, generally refers to a chemical reaction for obtaining a protein conjugate of the present disclosure that occurs specifically between a first functional moiety at specific positions on the protein (e.g. located in the oligosaccharide of the protein) and a second correspondingly complementary functional moiety linked to a molecule to be introduced under a suitable condition. The first functional moiety and the second complementary functional moiety are a bioorthogonal ligation reaction pair. Generally, the first functional moiety at the specific positions on the protein would be easily distinguished from other groups on the other parts of the protein. Generally, the second complementary functional moiety would not react with the other parts of the protein except for the first functional moiety at the specific positions. For example, an azido group is a functional moiety capable of participating in a bioorthogonal ligation reaction. A complementary DBCO or BCN group could specifically react with the azido group without cross-reacting with other groups on the protein. In another example, a —NH₂ group may not be a functional moiety capable of participating in a bioorthogonal ligation reaction in the present disclosure, as there are many —NH₂ groups at different sites of a protein, which cannot be distinguished from each other by using a N-hydroxysuccinimide ester reagent. A skilled person in the art will understand that if a—NH₂ group at a specific position of the protein could be easily distinguished from the other —NH₂ groups on the other part of the protein, for example, under certain conditions, then the —NH₂ group at such specific positions of the protein may also be a functional moiety capable of participating in a bioorthogonal ligation reaction. Many chemically reactive functional moieties with suitable reactivity, chemo selectivity and/or biocompatibility can be used in a bioorthogonal ligation reaction. A functional moiety capable of participating in a bioorthogonal ligation reaction could be selected from, but not limited to, the following: azido groups, terminal alkynyl groups, cyclic alkynyl groups, tetrazinyl groups, 1,2,4-trazinyl groups, terminal alkenyl groups, cyclic alkenyl groups, ketone groups, aldehyde groups, hydroxyl amino groups, sulfydryl groups, N-maleimide groups and their functional derivatives (see Bertozzi C. R., et. al Angew. Chem. Int. Ed., 2009, 48, 6974; Chin J. W., et. al ACS Chem. Biol. 2014, 9, 16; van Del F. L., et. al Nat. Commun., 2014, 5, 5378; Prescher J. A., et. al Acc. Chem. Res. 2018, 51, 1073; Devaraj NK. ACS Cent. Sci. 2018, 4, 952; Liskamp R. M. J., et. al Chem. Sci., 2020, 11, 9011). The functional derivatives of the above functional moiety may retain similar or higher reactivities of the functional moiety that they derive from in a bioorthogonal ligation reaction.

As used herein, the term “functional variant” of a parent polypeptide or protein generally refers to a polypeptide or protein having substantial or significant sequence identity or similarity to a parent polypeptide or protein, and retains at least one of the functions of the parent polypeptide or protein of which it is a variant. For example, a functional variant of an enzyme retains the enzymatic activity to a similar extent, the same extent, or to a higher extent, as the parent enzyme. In reference to the parent polypeptide or protein, the functional variant can, for instance, be about 80% or more, about 90% or more, about 95% or more, about 96% or more, about 97% or more, about 98% or more, or about 99% or more identical in amino acid sequence. In some cases, the functional variant may be a polypeptide different from the parent protein or polypeptide by at least one amino acid. For example, the functional variant may be a polypeptide different from the parent polypeptide or protein by an addition, deletion or substitution of one or more amino acid, such as 1-200, 1-100, 1-50, 1-40, 1-30, 1-20, 1-15, 1-14, 1-13, 1-12, 1-11, 1-10, 1-9, 1-8, 1-7, 1-6, 1-5, 1-4, 1-3, or 1-2 amino acid.

As used herein, the term “functional fragment” of a parent protein or polypeptide generally refers to a protein or polypeptide (including, but not limited to, an enzyme), which contains at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino acid residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, at least 250 contiguous amino acid residues or at least 350 contiguous amino acid residues of the parent polypeptide or protein, and retains at least one of the functions of the parent protein or polypeptide. For example, a “functional fragment” of a parent enzyme retains the enzymatic activity to a similar extent, the same extent, or to a higher extent, as the parent enzyme.

The term “fucosyltransferase”, as used herein, generally refers to an enzyme or a functional variant thereof that can transfer a L-fucose sugar from a fucose donor substrate (such as, guanosine diphosphate-fucose) to an acceptor substrate. The acceptor substrate can be another sugar such as a sugar comprising a GlcNAc-Gal (LacNAc), as in the case of N-glycosylation, or in the case of O-linked glycosylation. Examples of fucosyltransferase include α-1,3 fucosyltransferase. The term “fucosyltransferase” may comprise any functional fragments (e.g. a catalytic region thereof), or functional variants (e.g., mutant) of a parent enzyme (e.g., a wildtype fucosyltransferase). A “fucosyltransferase” of the present disclosure may be derived from any species, such as mammals (e.g., human), bacteria, nematodes or trematodes. In some embodiments, the “fucosyltransferase” is derived from bacteria. In some embodiments, the “fucosyltransferase” is derived from Helicobacter pylori.

The term “fusion tag”, as used herein, generally refers to a peptide fragment fused to a protein of interest. There are different types of fusion tags that can be used for different applications, for example, epitope tags, affinity tags, and fluorescent tags. Epitope tags are usually short peptide sequences that can be used for immunological applications, such as western blot and co-immunoprecipitation. Affinity tags are generally longer and are used for protein purification or increasing protein solubility (e.g., Hisx6 tags). Fluorescent tags can be used in both live and dead cells and are largely used for imaging studies, such as cellular localization and co-expression experiments.

The term “Fuc”, as used herein, generally refers to a fucose linked to a GlcNAc, wherein the GlcNAc is directly linked to an amino acid of a protein (e.g., an antibody or a fragment thereof). Preferably, the “Fuc” may be linked to the GlcNAc through an α1,6 linkage. In the present disclosure, the term Fuc is different from the term “Fuco”, as comprised in Fuc* of the present disclosure. In the present disclosure, the term “Fuco” generally refers to the

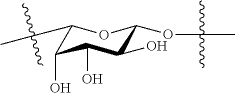

### of Fuc*.

The term “pharmaceutically active molecule”, as used herein, generally refers to a substance that is pharmaceutically effective. In the present disclosure, a fluorescent label may not be a pharmaceutically active molecule. For example, a pharmaceutically active molecule may be an agent capable of alleviating, treating, preventing a disease, or delaying the progress of a disease. The disease may be a disease associated with abnormal cell proliferation and/or cellular dysfunction. The disease may be a tumor and/or an immune disease. In some cases, the pharmaceutically active molecule comprises a cytotoxin. A cytotoxin may comprise any agent capable of interfering with cell proliferation and/or differentiation. A cytotoxin may have a cytotoxic effect on tumors including the depletion, elimination and/or the killing of tumor cells.

The term “corresponding antibody”, as used herein, generally refers to an antibody from which a protein conjugate can be obtained after certain modifications, e.g., glycosylation, ligation reaction or conjugation, especially after performing the method of the present disclosure. A protein conjugate may be capable of binding to the same antigen or the same antigen epitope with its corresponding antibody. A corresponding antibody can be conjugated with an active molecule to become a protein conjugate. For a given protein conjugate, if the protein conjugate can be obtained from an antibody by one or more steps of glycosylation, deglycosylation, and conjugation with an active molecule, the antibody may be the corresponding antibody of the protein conjugate derived therefrom. The “corresponding antibody” and the protein conjugate may have different glycoforms. For example, the “corresponding antibody” may be an antibody comprising heterogenous glycoforms (e.g. a mixture of G₂(F), G₁(F) and G₀(F)).

The term “spacer moiety”, as used herein, generally refers to a chemical structure capable of i) linking two parts together, ii) tuning the distance between the two parts that it links with, iii) tuning the hydrophilicity of the molecule comprising it, and/or iv) tuning the conformation of the molecule comprising it.

In the present disclosure,

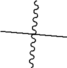

generally refers to a covalent bond linked to another moiety.

Unless otherwise specified, “a”, “an”, “the” and “at least one” are used interchangeably and refer to one or more than one.

In the present disclosure, the term “comprise” also encompasses “is”, “has” and “consist of”. For example, “a composition comprising X and Y” may be understood to encompass a composition that comprises at least X and Y. It also discloses a composition that only comprises X and Y (i.e., a composition consisting of X and Y).

### Protein Conjugate

In one aspect, the present disclosure provides a protein conjugate. The protein conjugate comprises a protein and an oligosaccharide comprising a structure of Formula (I):

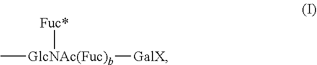

wherein: the GlcNAc is directly or indirectly linked to an amino acid of the protein; the GalX is a galactose or a substituted galactose (i.e. optionally, the galactose may be substituted, referred to as optionally substituted galactose); the Fuc is a fucose, and b is 0 or 1; the Fuc* is a fucose derivative comprising two or more active molecules (AM).

The GalX may be linked to the GlcNAc through a β1,4 linkage. For example, the C1 position of the GalX is linked to the C4 position of the GlcNAc through a —O—.

The Fuc may be linked to the GlcNAc through an α1,6 linkage. For example, the C1 position of the Fuc is linked to the C6 position of the GlcNAc through a —O—.

The Fuc* may be linked to the GlcNAc through an α1,3 linkage. For example, the C1 position of the Fuc* is linked to the C3 position of the GlcNAc through a —O—.

In some cases the Fuc* comprises the structure of Formula (II):

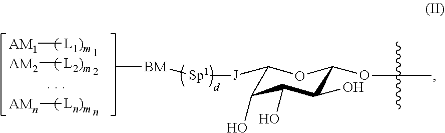

wherein: J is a jointer; Sp¹ is a spacer moiety, d is 0 or 1; BM is a branching moiety; L₁ to Lₙ each independently is a linker, m₁ to mₙ each independently is 0 or 1; AM₁ to AMₙ each independently is an active molecule; and n is an integer from 2-10. The various AMs (i.e., AM₁, AM₂ . . . , AMₙ) may be the same or may be different from each other. The various linkers (i.e., L₁, L₂, . . . , Lₙ) may be the same or may be different from each other. The J may be directly linked to the

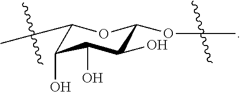

A branching moiety is a chemical structure capable of linking more than two parts together. The branching moiety BM may comprise

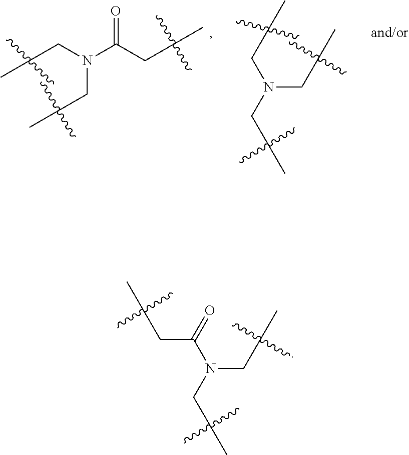 [SMILES: *CC(=O)N(C*)C* ; *CN(C*)C* ; *CC(=O)N(C*)C*]

For example, the BM may comprise one or more structures selected from

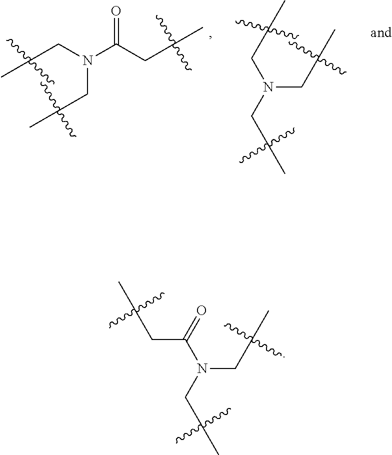 [SMILES: *CC(=O)N(C*)C* ; *CN(C*)C* ; *CC(=O)N(C*)C*]

The right side of the structure of BM may be linked (e.g., directly linked) to the Sp¹ or J. For example, when d is 0, the right side of the structure of BM is linked (e.g., directly linked) to J. When d is 1, the right side of the structure of BM is linked (e.g., directly linked) to Sp¹ and Sp¹ is in turn linked (e.g., directly linked) to J.

In some cases, n may be 2, and the Fuc* may comprise the structure of Formula (III):

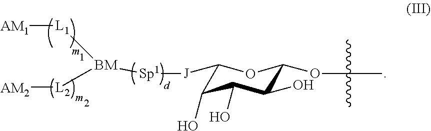

For example, the BM is selected from the group consisting of:

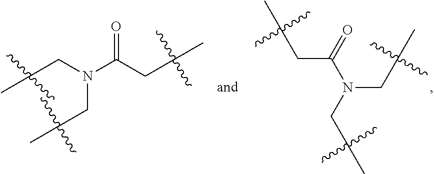 [SMILES: *CC(=O)N(C*)C* ; *CC(=O)N(C*)C*]

wherein the right side of the structure is directly linked to the Sp¹ or J. The jointer J may have a structure of:

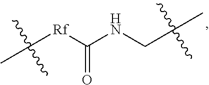

herein R_{f} is —CH₂—, —NH— or —O—. In some cases, the jointer J is

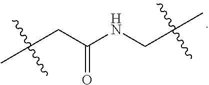 [SMILES: *CNC(=O)C*]

The right side of the structure of J may be linked (e.g., directly linked) to the left side of:

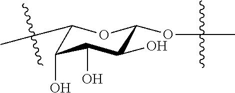

### of Formula (II).

The Sp¹ may be a structure selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs². For example, each of the alkylene, cycloalkylene, alkenylene, cycloalkenylene, alkynylene, cycloalkynylene, (hetero)arylene, (hetero)arylalkylene and alkyl(hetero)arylene may independently be substituted with one or more Rs¹. In some cases, one or more Rs² may be inserted in the alkylene, cycloalkylene, alkenylene, cycloalkenylene, alkynylene, cycloalkynylene, (hetero)arylene, (hetero)arylalkylene and/or alkyl(hetero)arylene. For example, an alkylene may be inserted by one or more —O— to become a -PEG-.

Each Rs¹ may independently be selected from the group consisting of halogen, —OH, —NH₂ and —COOH.

Each Rs² may independently be selected from the group consisting of —O—, —N—

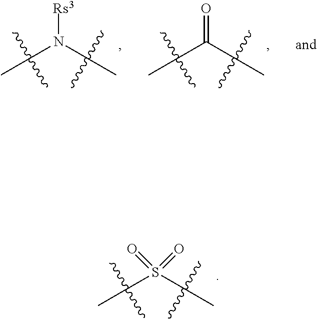

Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the Sp¹ may be selected from the group consisting of:

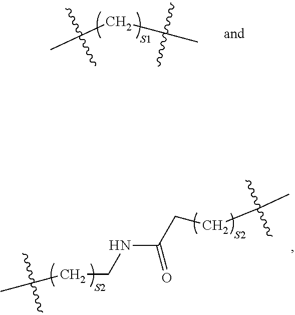

S1 may be an integer from 1-50 (for example, 1-40, 1-30, 1-20, 1-15, 1-14, 1-13, 1-12, 1-11, 1-10, 1-9, 1-8, 1-7, 1-6, 1-5, 1-4, 1-3, 1-2 or 1), each S2 may independently be an integer from 0-50 (for example, 0-40, 0-30, 0-20, 0-15, 0-14, 0-13, 0-12, 0-11, 0-10, 0-9, 0-8, 0-7, 0-6, 0-5, 0-4, 0-3, 0-2, 0-1 or 0). Each said —CH₂—(—CH₂— in the parentheses) may independently be replaced by a —O—, with the proviso that two or more consecutive —CH₂— are not simultaneously replaced by —O—. Accordingly, when one —CH₂— is replaced by a —O—, its immediate neighboring —CH₂— to the left and to the right may not be replaced by —O—. For example, the

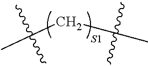

may be —(CH₂OCH₂)_{S1′}—, and the S1′ may be 0-20 (e.g., 0-15, 0-14, 0-13, 0-12, 0-11, 0-10, 0-9, 0-8, 0-7, 0-6, 0-5, 0-4, 0-3, 0-2, 0-1 or 0).

The right side of the structure of the Sp¹ may be linked to the J and the left side of the structure of the Sp¹ may be linked to the BM.

In some cases, the Sp¹ may be

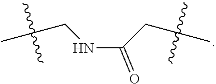 [SMILES: *CNC(=O)C*]

In some cases, the Sp¹ may be

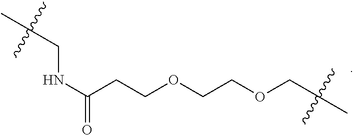 [SMILES: *CNC(=O)CCOCCOC*]

The right side of the structure of the Sp¹ may be linked to the J and the left side of the structure of the Sp¹ may be linked to the BM.

In some cases, d is 0 (meaning that the Sp¹ is absent), and the BM is directly linked to the J. For example, the FD4, FD5 and FD6 of FIG. 2 comprise a J of:

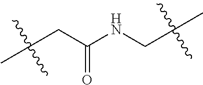 [SMILES: *CNC(=O)C*]

### and a BM of:

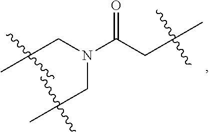 [SMILES: *CC(=O)N(C*)C*]

and the BM is directly linked to the J.

In the protein conjugate of the present disclosure, each of L₁ to Lₐ may independently be a linker of Formula (IV): (CL)_{y}-(FL)ₓ (IV). The various L (i.e., L₁, L₂ . . . , Lₙ) may be the same or may be different from each other. FL is a spacer moiety, x is 0 or 1, CL is a cleavable linker, y is 0 or 1, the right side of Formula (IV) is linked to said BM and the left side of Formula (IV) is linked to said AM. For example, when x is 1 and y is 1, the FL side is linked to the BM and the CL side is linked to the AM.

The FL may be a spacer moiety selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs². For example, one or more of the alkylene, cycloalkylene, alkenylene, cycloalkenylene, alkynylene, cycloalkynylene, (hetero)arylene, (hetero)arylalkylene and alkyl(hetero)arylene may be substituted by one or more Rs¹. In some cases, one or more Rs² may be inserted in the alkylene, cycloalkylene, alkenylene, cycloalkenylene, alkynylene, cycloalkynylene, (hetero)arylene, (hetero)arylalkylene and/or alkyl(hetero)arylene. For example, an alkylene may be inserted by one or more —O— to become a -PEG-.

Each Rs¹ may independently be selected from the group consisting of halogen, —OH, —NH₂ and —COOH, and each Rs² may independently be selected from the group consisting of —O—, —S—,

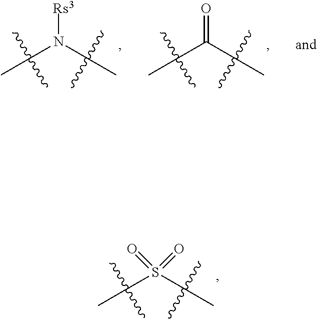

Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the FL is a spacer moiety selected from the group consisting of:

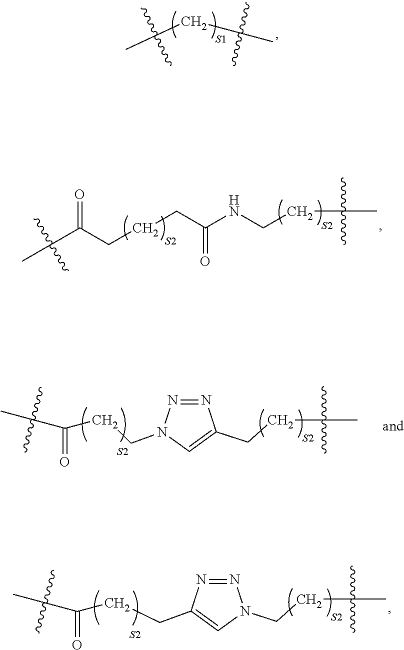

wherein said S1 may be independently an integer from 1-50, said S2 may be independently an integer from 0-50. Each said —CH₂—(—CH₂— in the parentheses) may independently be replaced by a —O—, with the proviso that two or more consecutive —CH₂— are not simultaneously replaced by —O—. Accordingly, when one —CH₂— is replaced by a —O—, its immediate neighboring —CH₂— to the left and to the right may not be replaced by —O—. The right side of the structure of the FL may be linked to the BM, and the left side of the structure of the FL may be linked to the CL or the AM.

In some cases, the FL is a spacer moiety selected from the group consisting of:

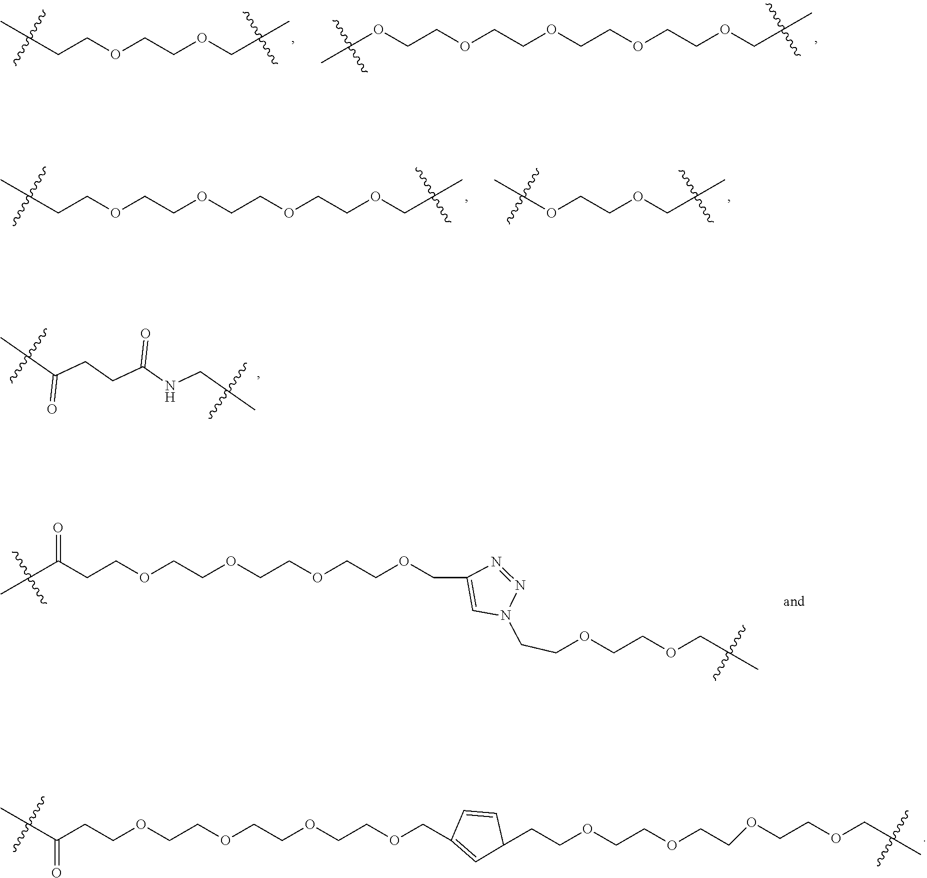

The right side of the structure of the FL may be linked to the BM, and the left side of the structure of the FL may be linked to the CL or the AM.

In some cases (e.g., for some L, i.e., L₁, L₂ . . . , and/or Lₙ), x is 0 (meaning that FL is absent), and y is 1, the CL is linked (e.g., directly linked) to the corresponding AM and the BM. For example, the right side of CL is linked to the BM and the left side of the CL is linked to the AM.

In some cases (e.g., for some L, i.e., L₁, L₂ . . . , and/or Lₙ), y is 0 (meaning that CL is absent), and x is 1, the FL is linked (e.g., directly linked) to the corresponding AM and the BM. For example, the right side of FL is linked to the BM and the left side of the FL is linked to the AM.

In some cases, both x and y are 0, meaning that the specific L is absent, and the corresponding AM may be directly linked to the BM.

In some cases, both x and y are 1, the FL is (directly) linked to the CL and the BM, and the CL is in turn (directly) linked to the corresponding AM. For example, the right side of FL is linked to the BM and the left side of the CL is linked to the AM.

The CL may be an acid-labile linker, a redox-active linker, a photo-active linker and/or a proteolytically cleavable linker. In some cases, the CL may be a vc-PAB-linker and/or a GGFG-linker.

In the present disclosure, each of AM₁ to AMₙ may independently be a chemically active molecule, an enzymatically active molecule, a biologically active molecule, and/or a pharmaceutically active molecule. For example, one AM may be the same as another AM, or different AMs may be different from each other. Each AM may independently be a chemically active molecule, an enzymatically active molecule, a biologically active molecule, or a pharmaceutically active molecule.

In some cases, the AM₁ to AMₙ independently comprises a chemically or enzymatically active molecule X_{F}. For example, AM₁ to AMₙ may comprise one or more X_{F}. The chemically or enzymatically active molecule X_{F} may comprise a functional moiety capable of participating in a ligation reaction. For example, the X_{F} may comprise a functional moiety capable of participating in a bioorthogonal ligation reaction.

In some cases, the X_{F} may comprise a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof:

In some cases, the X_{F} may comprise a functional moiety selected from the group consisting of:

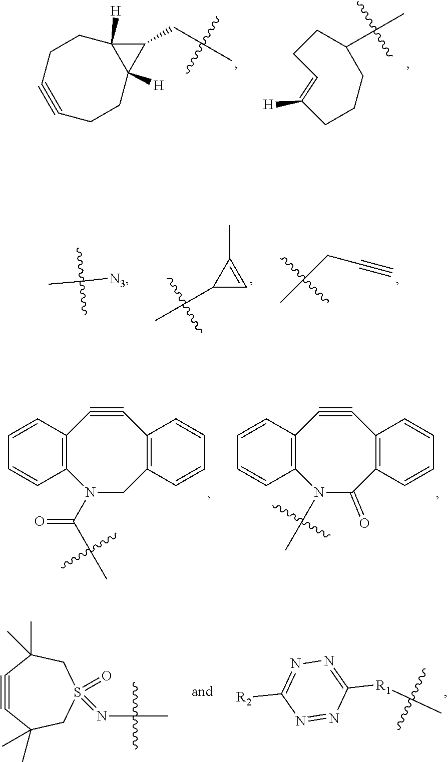

Wherein R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group.

In some cases, the X_{F} comprises a functional moiety selected from the group consisting of:

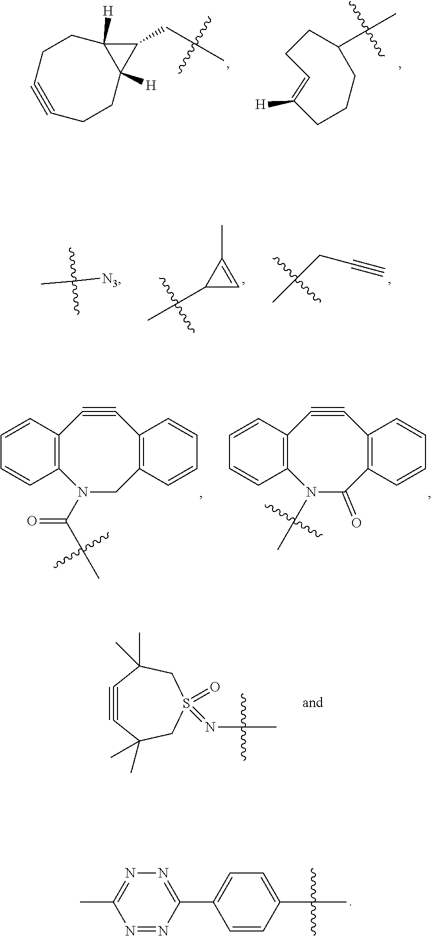

In some cases, the AM₁ to AMₙ independently comprises a biologically active molecule and/or pharmaceutically active molecule P_{F}. For example, AM₁ to AMₙ may comprise one or more P_{F}. The P_{F} may comprise a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof:

In some cases, the P_{F} is a pharmaceutically active molecule.

For example, the P_{F} may comprise a cytotoxin, an agonist, an antagonist, an antiviral agent, an antibacterial agent, an oligonucleotide, a polypeptide or any combination thereof: In some cases, the P_{F} comprises a cytotoxin or an agonist (such as a sting agonist, or a toll like receptor (such as TLR7/8) agonist).

In some cases, the P_{F} comprises a DNA or RNA damaging agent, an RNA polymerase inhibitor, a topoisomerase inhibitor and/or a microtubule inhibitor.

In some cases, the P_{F} comprises a pyrrolobenzodiazepine, an auristatin, a maytansinoids, a duocarmycin, a tubulysin, an enediyene, a doxorubicin, a pyrrole-based kinesin spindle protein inhibitor, a calicheamicin, an amanitin, a camptothecin and/or derivatives thereof:

In some cases, the P_{F} comprises an MMAE, a DXd, T785 and/or their derivatives thereof:

In the present disclosure, the GalX may be a galactose, or a substituted galactose. In some cases, the GalX is a monosaccharide (e.g., after substitution, the substituted GalX is still a monosaccharide, for example, the substituted GalX only comprise one monosaccharide unit, for example, GalNAz is a monosaccharide).

In some cases, the GalX may be a galactose.

In some cases, the GalX may be a substituted galactose, and the hydroxyl group at one or more positions selected from the C2 position, the C3 position, the C4 position and the C6 position of the galactose is substituted. For example, the GalX may be a substituted galactose, wherein the hydroxyl group at the C2 position of the galactose is substituted.

In some cases, the GalX may be a galactose substituted by

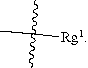

The Rg¹ may be selected from the group consisting of hydrogen, halogen, —NH₂, —SH, —N₃, —COOH, —CN, C₁-C₂₄ alkyl, C₃-C₂₄ cycloalkyl, C₂-C₂₄ alkenyl, C₅-C₂₄ cycloalkenyl, C₂-C₂₄ alkynyl, C₆-C₂₄ cycloalkynyl, C₂-C₂₄ (hetero)aryl, C₃-C₂₄ alkyl(hetero)aryl, C₃-C₂₄ (hetero)arylalkyl and any combination thereof: Each of the alkyl, cycloalkyl, alkenyl, cycloalkenyl, alkynyl, cycloalkynyl, (hetero)aryl, alkyl(hetero)aryl and (hetero)arylalkyl may independently be substituted by one or more Rs⁴ and/or may independently be interrupted by one or more Rs⁵. For example, one or more of the alkyl, cycloalkyl, alkenyl, cycloalkenyl, alkynyl, cycloalkynyl, (hetero)aryl, alkyl(hetero)aryl and (hetero)arylalkyl may independently be substituted by one or more Rs⁴. In some cases, one or more Rs⁵ may be inserted in the alkyl, cycloalkyl, alkenyl, cycloalkenyl, alkynyl, cycloalkynyl, (hetero)aryl, alkyl(hetero)aryl or (hetero)arylalkyl. For example, the alkyl may be inserted by one or more —O— to become a -PEG.

Each Rs⁴ may independently be selected from the group consisting of halogen, —OH, —NH₂, —SH, —N₃, —COOH and —CN. Each Rs⁵ may independently be selected from the group consisting of —O—, —S—,

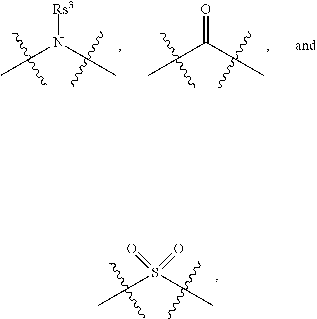

and N and Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the GalX may be a galactose substituted by

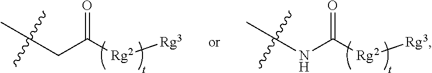

wherein t is 0 or 1, Rg² is selected from the group consisting of C₁-C₂₄ alkylene, C₃-C₂₄ cycloalkylene, C₂-C₂₄ alkenylene, C₅-C₂₄ cycloalkenylene, C₂-C₂₄ alkynylene, C₆-C₂₄ cycloalkynylene, C₂-C₂₄ (hetero)arylene, C₃-C₂₄ alkyl(hetero)arylene and C₃-C₂₄ (hetero)arylalkylene. Each of the alkylene, cycloalkylene, alkenylene, cycloalkenylene, alkynylene, cycloalkynylene, (hetero)arylene, alkyl(hetero)arylene and (hetero)arylalkylene may independently be substituted by one or more Rs⁴ and/or may independently be interrupted by one or more Rs⁵. For example, one or more of the alkylene, cycloalkylene, alkenylene, cycloalkenylene, alkynylene, cycloalkynylene, (hetero)arylene, alkyl(hetero)arylene and (hetero)arylalkylene may independently be substituted by one or more Rs⁴. In some cases, one or more Rs⁵ may be inserted in the alkylene, cycloalkylene, alkenylene, cycloalkenylene, alkynylene, cycloalkynylene, (hetero)arylene, (hetero)arylalkylene or alkyl(hetero)arylene. For example, alkylene may be inserted by one or more —O— to become a -PEG-.

Rg³ may be selected from the group consisting of hydrogen, halogen, —OH, —NH₂, —SH, —N₃, —COOH, —CN, C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl, C₂-C₂₄ (hetero)aryl and any combination thereof, wherein each of the C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl and C₂-C₂₄ (hetero)aryl may independently be substituted by one or more Rs⁴.

Each Rs⁴ may independently be selected from the group consisting of halogen, —OH, —NH₂, —SH, —N₃, —COOH and —CN.

Each Rs⁵ may independently be selected from the group consisting of —O—, —S—,

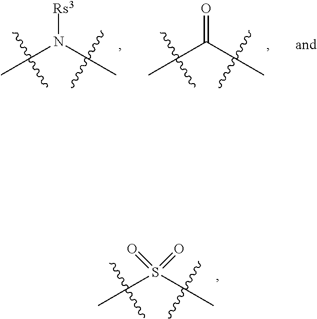

wherein Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the GalX may comprise a chemically and/or enzymatically active molecule X_{G}. The X_{G} may comprise a functional moiety capable of participating in a ligation reaction. The X_{G} may comprise a functional moiety capable of participating in a bioorthogonal ligation reaction. The X_{G} may comprise a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof. For example, the X_{G} may comprise a

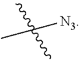 [SMILES: *N=[N+]=[N-]]

In some cases, when one or more of the AMs comprises a X_{F}, and the GalX comprises a X_{G}, the X_{G} does not substantially react with any X_{F}. For example, the X_{G} may comprise a

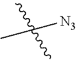 [SMILES: *N=[N+]=[N-]]

and the X_{F} may comprise a

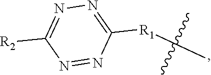

R₁ and R₂ are as defined in the present disclosure. In some cases, the X_{G} and the X_{F} may comprise the same functional moiety. For example, the X_{G} may comprise a

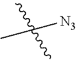 [SMILES: *N=[N+]=[N-]]

and the X_{F} may comprise a

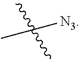 [SMILES: *N=[N+]=[N-]]

In some cases, the GalX is selected from the group consisting of:

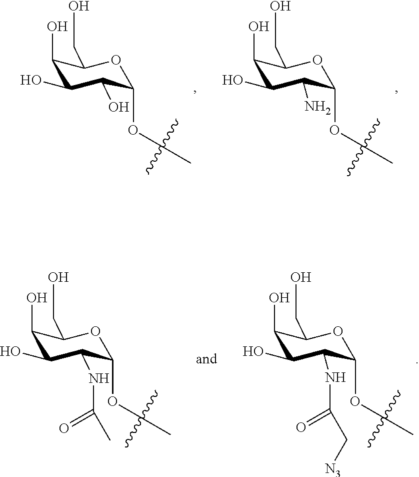

In the protein conjugate of the present disclosure, the protein may comprise an antigen binding fragment and/or an Fc fragment. The Fc fragment may be an IgG Fc fragment. In some cases, the oligosaccharide may be linked to the Fc fragment. For example, the oligosaccharide may be linked to the CH₂ domain of the Fc fragment.

The oligosaccharide of the protein conjugate may be an N-linked oligosaccharide. The oligosaccharide may be linked to an Asparagine (Asn) residue of the protein. For example, the GlcNAc of Formula (I) may be directly linked to an Asn residue of the protein.

In some cases, the oligosaccharide may be linked to the Asn297 of the Fc fragment, numbered according to the Kabat numbering system.

In some cases, the GlcNAc of Formula (I) may be linked to a saccharide of the oligosaccharide. For example, the GlcNAc of Formula (I) may be linked to a mannose of the oligosaccharide, and b may be 0.

The protein of the present disclosure may be an antibody. For example, the protein of the present disclosure may be a monoclonal antibody. In some cases, the protein of the present disclosure may be an IgG antibody. In some cases, the protein of the present disclosure may be a humanized antibody. For example, the protein of the present disclosure may be a nanobody, ScFv, Fab, F(ab)₂, F(ab′) and/or F(ab′)₂.

In the present disclosure, the protein of the protein conjugate may comprise a Fc fragment and an antigen binding fragment. The protein may be an antibody or a fragment thereof. For example, the antibody may recognize a target antigen. In some embodiments, the target antigen is a tumor antigen and may be localized to a tumor cell's surface. In some cases, the antibody bound to the target antigen can be internalized after binding to the tumor cell. When the antibody is covalently linked to an active molecule, the active molecule can be released into the cell after internalization. For example, when the functionalized antibody is linked to a cytotoxic drug, the cytotoxic drug can be released into the cell after internalization, resulting in cell death. In some cases, the target antigen displays differential expression between normal cells and tumor cells, displaying increased expression on tumor cells. For example, the target antigen may be Her2, Her3, Trop2, EGFR, BCMA, Nectin-4, MUC1, c-Met, PSMA, GD2, GPC3, CEA, CD20, ErbB3, ErbB4, PD-L1 and/or EpCAM. For example, the target antigen may be Trop2 or Her2.

In the present disclosure, the protein may be an antibody or a fragment thereof. For example, the antibody could be but not limited to trastuzumab, bevacizumab, rituximab, durvalumab, pertuzumab, raxibacumab, dinutuximab, ixekizumab, labetuzumab, odesivimab. risankizumab, dinutuximab, adalimumab, cetuximab, daratumumab, tocilizumab and hRS7. For example, the antibody may be trastuzumab or hRS7.

For example, the heavy chain of trastuzumab may comprise the amino acid sequence as set forth in SEQ ID NO: 9, and the light chain of trastuzumab may comprise the amino acid sequence as set forth in SEQ ID NO. 8. For example, the heavy chain of hRS7 may comprise the amino acid sequence as set forth in SEQ ID NO: 11, and the light chain of hRS7 may comprise the amino acid sequence as set forth in SEQ ID NO: 10.

In the present disclosure, the protein conjugate may have the similar binding affinity towards an antigen, compared to the corresponding antibody. For example, the protein may be an antibody, and the protein conjugate may have a comparable binding activity towards an antigen, compared to the corresponding antibody.

For example, the binding activity or binding affinity to an antigen of the protein conjugate in the present disclosure may be about 0.1% to about 100000% (e.g., about 1%-10000%, about 10%-100⁰%, or about 50%-200%) of the binding activity or binding affinity of the corresponding antibody. The binding activity to an antigen may be compared by a quantitative or a non-quantitative method. In some cases, the binding activity or binding affinity can be qualitative. For example, by ELISA, under a condition that allows the protein conjugate and/or its corresponding antigen to bind to a target (e.g., an antigen), contact the protein conjugate and/or its corresponding antigen with the target (e.g,, the antigen), determine whether a complex is formed between the protein conjugate and the target (e.g., an antigen), and determine whether a complex is formed between the corresponding antibody and the target (e.g., an antigen). For example, the binding activity or binding affinity to a target (e.g., an antigen) may be quantified by a value. For example, the value may be a Kd value. For example, the value is an OD value. For example, the value is an absorbance value. The binding affinity can be qualified by the value (e.g., OD value, KD value, or absorbance value) after statistical analysis, in which the binding affinity of the corresponding antibody may be set as 100%.

In the present disclosure, a variety of methods can be used to determine the binding activity of the protein conjugate and its corresponding antigen. The binding activity can be determined by, for example, ELISA, isothermal titration calorimetry, surface plasmon resonance, and/or biolayer interferometry. For example, the binding activity of the corresponding antibody can be set as 100%.

In some cases, the protein of the protein conjugate is a Fc-fusion protein. The Fc-fusion protein may comprise a Fc fragment and a biologically active protein or polypeptide. For example, the biologically active protein or polypeptide may be therapeutically effective. For example, the biologically active protein may be derived from a non-immunoglobulin protein. For example, the biologically active protein may be a cytokine, a complement, and/or an antigen, or a fragment thereof:

In some cases, in the protein conjugate of the present disclosure, n is 2, the Fuc* comprises AM₁ and AM₂, both the AM₁ and the AM₂ comprises a X_{F}, the X_{F} of the AM₁ and the X_{F} of the AM₂ may be identical or different.

In some cases, in the protein conjugate of the present disclosure, n is 2, the Fuc* comprises AM₁ and AM₂, both the AM₁ and the AM₂ comprises a X_{F}, the X_{F} of the AM₁ and the X_{F} of the AM₂ is independently selected from the group consisting of:

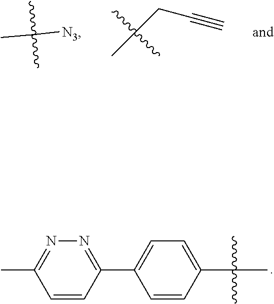

However, the X_{F} of AM₁ may not react bioorthogonally with the X_{F} of AM₂. For example, when the X_{F} of the AM₁ is

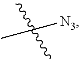 [SMILES: *N=[N+]=[N-]]

the X_{F} of the AM₂ shall not be

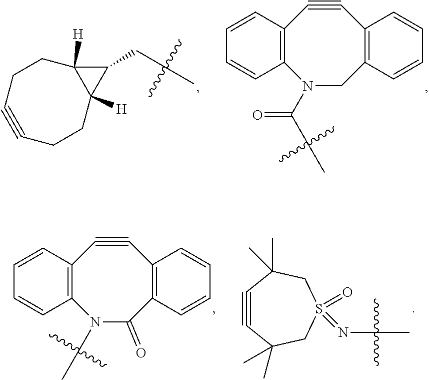

In another example, when the X_{F} of the AM₁ is

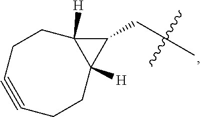 [SMILES: *C[C@H]1[C@H]2CCC#CCC[C@@H]12]

the X_{F} of the AM₂ shall not be

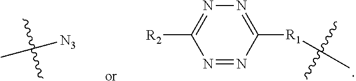

In another example, when the X_{F} of the AM₁ is

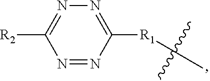

the X_{F} of the AM₂ shall not be

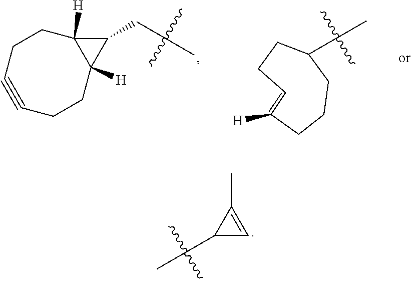

In some cases, in the protein conjugate of the present disclosure, n is 2, the Fuc* comprises AM₁ and AM₂, the AM₁ may comprise a X_{F} and the AM₂ may comprise a P_{F}; or the AM₁ may comprise a P_{F} and the AM₂ may comprise a X_{F}.

In some cases, in the protein conjugate of the present disclosure, n is 2, the Fuc* comprises AM₁ and AM₂, both the AM₁ and the AM₂ comprises a P_{F}, the P_{F} of AM₁ and the P_{F} of AM₂ are identical or different.

In some cases, the protein conjugate of the present disclosure comprises 1-20 (e.g., 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, or 1-20) of the structure of:

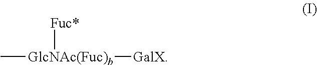

In some cases, the protein conjugate of the present disclosure comprises 2 or 4 of the structure of:

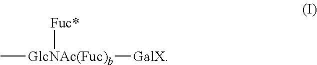

In some cases, the protein conjugate of the present disclosure comprises 2 of the structure of:

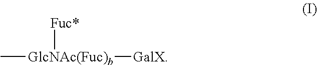

In some cases, the protein conjugate of the present disclosure has a structure of Formula (V):

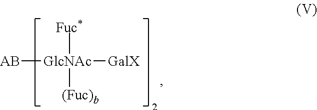

wherein AB is an antibody comprising a Fc fragment or a Fc-fusion protein, the GlcNAc is directly linked to an Asn of the Fc fragment of the AB, the Fuc is linked to the GlcNAc through an α-1,6 linkage, the GalX is linked to the GlcNAc through a β1,4 linkage, the Fuc* is linked to the GlcNAc through an α1,3 linkage and b is 0 or 1. In some case, b is 0. For example, when b is 0, the structure of Formula (V)

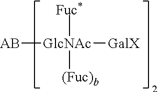

may be

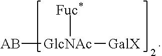

In some case, b is 1. In some case, the GlcNAc is directly linked to the N297 of the Fc fragment of the AB.

In some cases, the protein conjugate of the present disclosure comprises 4 of the structure of:

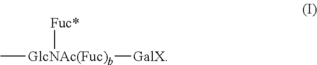

In some cases, the protein conjugate of the present disclosure has a structure of Formula (VI):

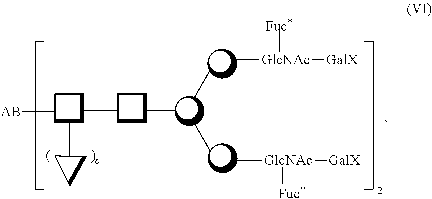

wherein AB is an antibody comprising a Fc fragment or a Fc-fusion protein, [symbol] is a GlcNAc, [symbol] is a mannose, [symbol] is a fucose linked to the [symbol] through a α1,6 linkage, c is 0 or 1; the oligosaccharide

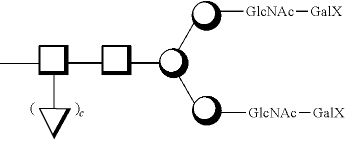

is linked to an Asn of the Fc fragment of the AB through the [symbol] , the GalX is linked to the neighboring GlcNAc through a β1,4 linkage, and the Fuc* is linked to the GlcNAc through an α1,3 linkage. In some cases, the oligosaccharide

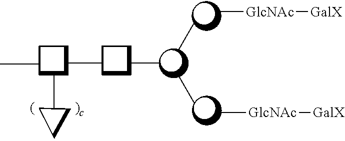

is linked to the N297 Fc fragment of the AB through the

[symbol]

In the present disclosure the Fuc* may be selected from the group consisting of:

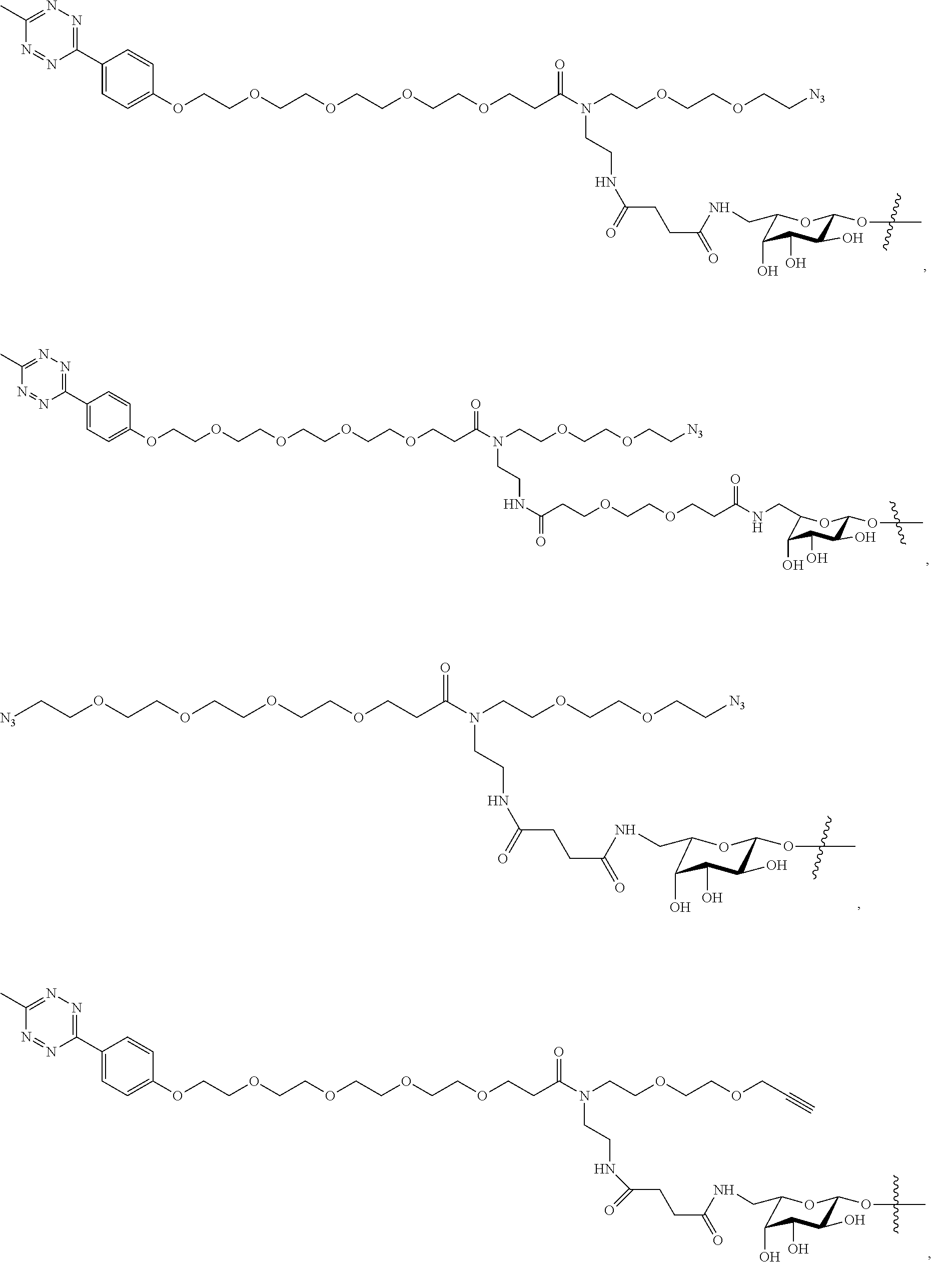

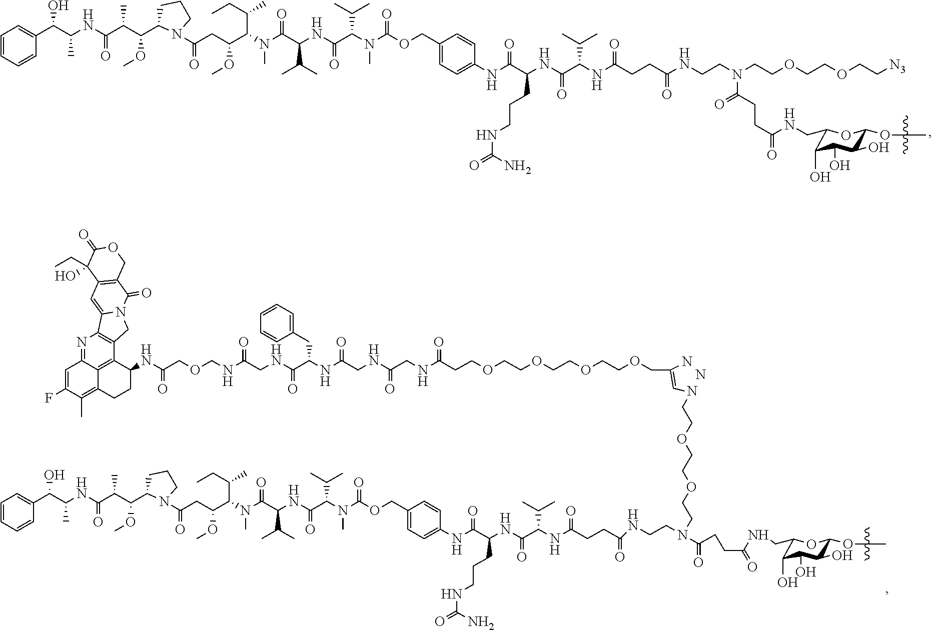

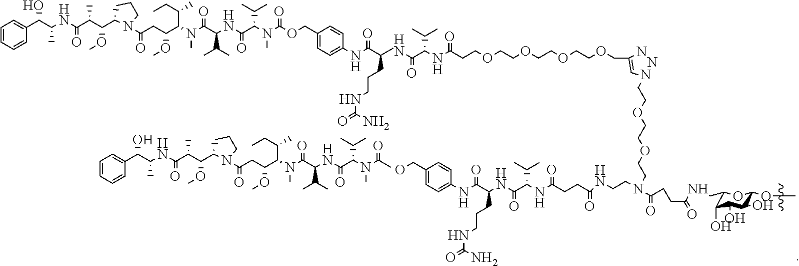

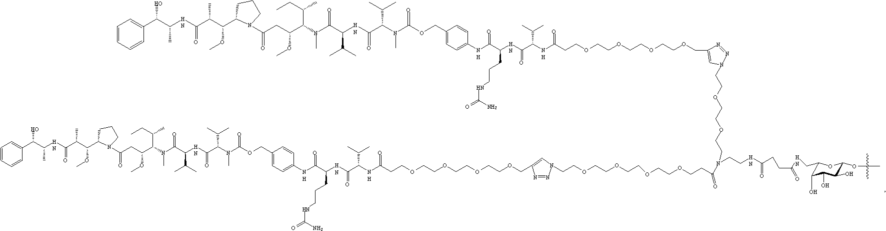

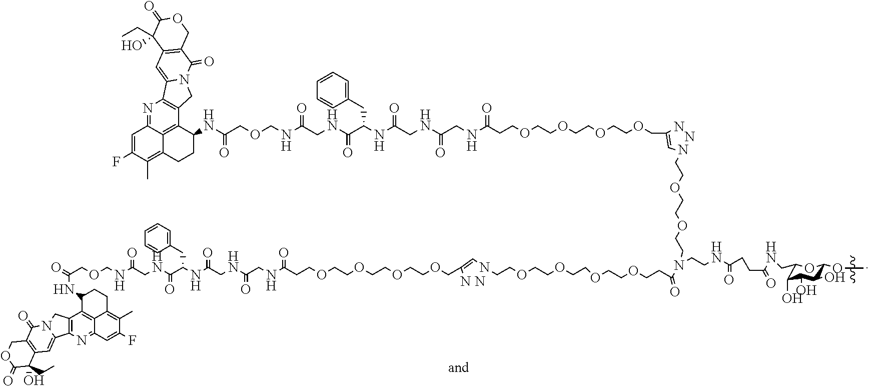

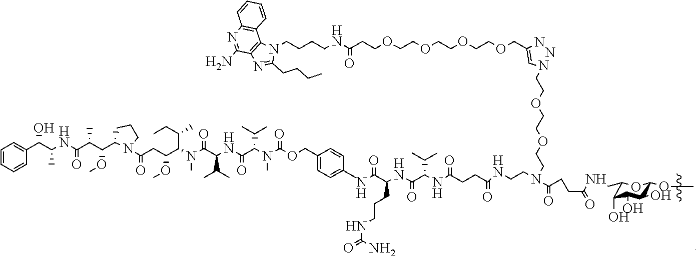

In another aspect, the present disclosure provides a protein conjugate, which could be obtained by reacting the protein conjugate of the present disclosure with one or more Y-(L′)ₑ-P_{F}′, wherein the Y comprises a functional moiety capable of reacting with the X_{F} and/or the X_{G}, L′ is a linker, e is 0 or 1, and the P_{F}′ is a biologically active molecule and/or a pharmaceutically active molecule. L′ is a linker that links the Y to the P_{F}′.

The P_{F}′ may be the same or different as the P_{F} of the present disclosure. The L′ may be the same or different as any of the L₁ to Lₙ of the present disclosure. For example, in the present disclosure, the P_{F}′ may be a different molecule than the P_{F}, but they can be selected from the same group of molecules. Similarly, the L′ may be a different linker structure than any of the L₁ to Lₙ.

For example, The P_{F}′ may comprise a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof: In some cases, the P_{F}′ is a pharmaceutically active molecule. For example, the P_{F}′ may comprise a cytotoxin, an agonist, an antagonist, an antiviral agent, an antibacterial agent, an oligonucleotide, a polypeptide or any combination thereof: In some cases, the P_{F}′ comprises a cytotoxin or an agonist (such as a sting agonist, or a toll like receptor (such as TLR7/8) agonist). In some cases, the P_{F}′ comprises a DNA or RNA damaging agent, an RNA polymerase inhibitor, a topoisomerase inhibitor and/or a microtubule inhibitor. In some cases, the P_{F}′ comprises a pyrrolobenzodiazepine, an auristatin, a maytansinoids, a duocarmycin, a tubulysin, an enediyene, a doxorubicin, a pyrrole-based kinesin spindle protein inhibitor, a calicheamicin, an amanitin, a camptothecin and/or derivatives thereof. In some cases, the P_{F}′ comprises an MMAE, a DXd, T785 and/or their derivatives thereof:

For example, the L′ may be a linker of Formula (X): (FL′)_{x′}, —(CL′)_{y′} (IV), the FL′ is a spacer moiety, the CL′ is a cleavable linker, x′ and y′ are independently 0 or 1. The right side of Formula (X) is linked to the Y, and the left side of Formula (X) is linked to the P_{F}′. For example, the FL′ side is linked to the Y, the CL′ side is linked to the P_{F}′.

The FL′ may be a spacer moiety selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs². For example, one or more of the alkylene, cycloalkylene, alkenylene, cycloalkenylene, alkynylene, cycloalkynylene, (hetero)arylene, (hetero)arylalkylene and alkyl(hetero)arylene may be substituted by one or more Rs¹. In some cases, one or more Rs² may be inserted in the alkylene, cycloalkylene, alkenylene, cycloalkenylene, alkynylene, cycloalkynylene, (hetero)arylene, (hetero)arylalkylene and/or alkyl(hetero)arylene. For example, an alkylene may be inserted by one or more —O— to become a -PEG-.

Each Rs¹ may independently be selected from the group consisting of halogen, —OH, —NH₂ and —COOH, and each Rs² may independently be selected from the group consisting of —O—, —S—,

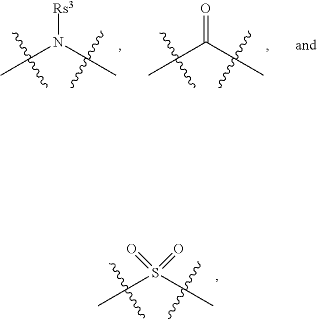

Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the FL′ is a spacer moiety selected from the group consisting of:

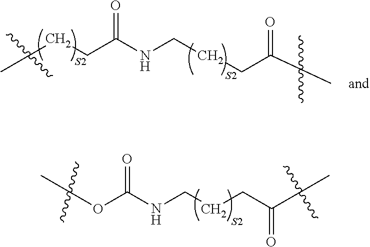

wherein each S2 may independently be 0-50 (for example, 0-40, 0-30, 0-20, 0-15, 0-14, 0-13, 0-12, 0-11, 0-10, 0-9, 0-8, 0-7, 0-6, 0-5, 0-4, 0-3, 0-2, or 0-1). Each said —CH₂—(—CH₂— in the parentheses) may independently be replaced by a —O—, with the proviso that two or more consecutive —CH₂— are not simultaneously replaced by —O—. Accordingly, when one —CH₂— is replaced by a —O—, its immediate neighboring —CH₂— to the left and to the right may not be replaced by —O—. The left side of the structure of the FL′ may be linked to the Y, and the right side of the structure of the FL may be linked to the CL or the P_{F}′.

In some cases, the FL′ is a spacer moiety selected from the group consisting of:

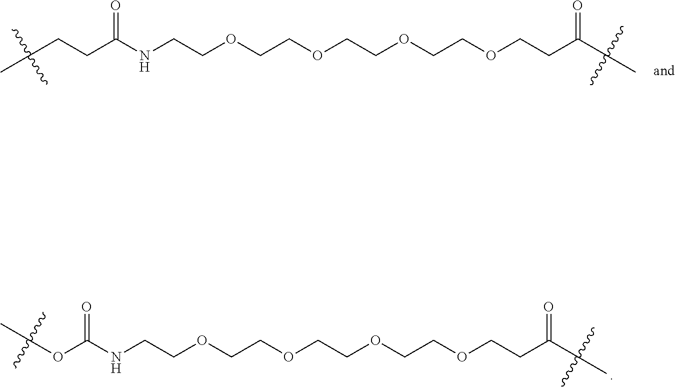 [SMILES: *CCC(=O)NCCOCCOCCOCCOCCC(*)=O ; *OC(=O)NCCOCCOCCOCCOCCC(*)=O]

The left side of the structure of the FL′ may be linked to the Y, and the right side of the structure of the FL may be linked to the CL or the P_{F}′.

The CL′ may be an acid-labile linker, a redox-active linker, a photo-active linker and/or a proteolytically cleavable linker. In some cases, the CL may be a vc-PAB-linker and/or a GGFG-linker.

In the present disclosure, Y may comprise a functional moiety capable of bioorthogonally reacting with the X_{F} and/or the X_{G} of the present disclosure. In some cases, Y is a functional moiety capable of bioorthogonally reacting with the X_{F} and/or the X_{G} of the present disclosure. In some cases, the Y only bioorthogonally reacts with the X_{F}. In some cases, the Y only bioorthogonally reacts with the X_{G}. In some cases, the Y bioorthogonally reacts with both the X_{F} and the X_{G}.

For example, Y may comprise a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. The functional derivatives may retain similar or higher activities as the above functional moieties in a bioorthogonal ligation reaction.

In some cases, the Y may comprise a functional moiety selected from the group consisting of:

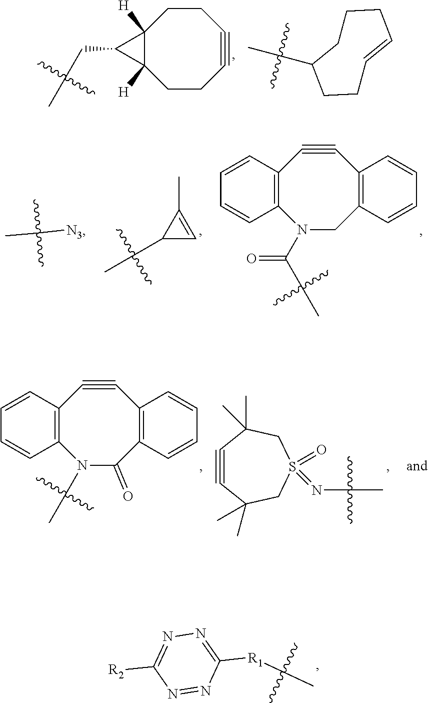

wherein each of R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group.

In some cases, the Y may comprise a functional moiety selected from the group consisting of:

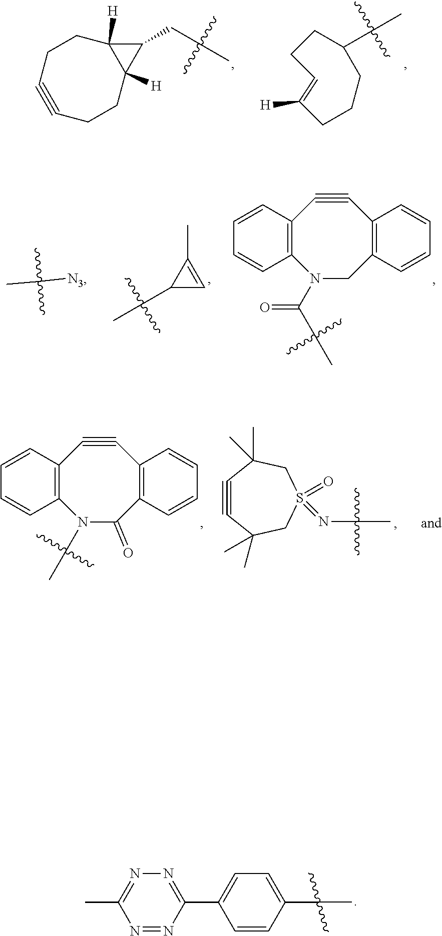

When the protein conjugate of the present disclosure reacts with multiple Y-(L′)ₑ-P_{F}′, the Y, L′, e, and P_{F}′ in different Y-(L′)ₑ-P_{F}′ may independently be identical or different. For example, some of the Y may be capable of only bioorthogonally reacting with X_{F}, some of the Y may be capable of only bioorthogonally reacting with the X_{G}, and some of the Y may be capable of bioorthogonally reacting with both the X_{F} and the X_{G}.

For example, when the X_{F} and/or the X_{G} comprise

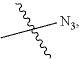 [SMILES: *N=[N+]=[N-]]

Y may comprise

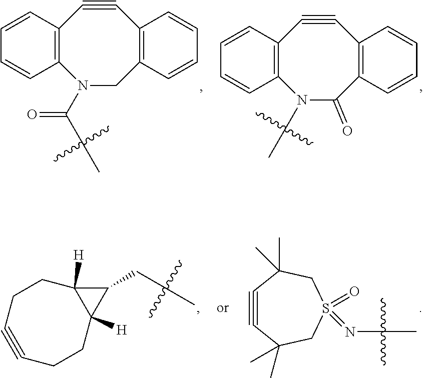

For example, when the X_{F} and/or the X_{G} comprise

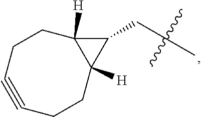 [SMILES: *C[C@H]1[C@H]2CCC#CCC[C@@H]12]

Y may comprise the

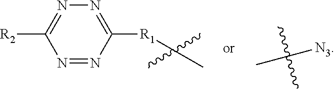

For example, when the X_{F} and/or the X_{G} comprise

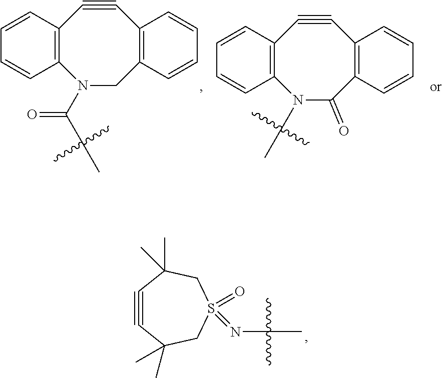

Y may comprise

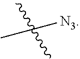 [SMILES: *N=[N+]=[N-]]

For example, when the X_{F} and/or the X_{G} comprise

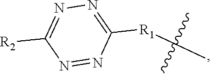

Y may comprise

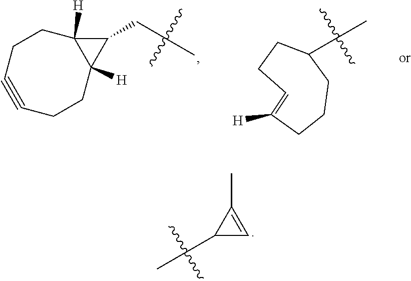

For example, when the X_{F} and/or the X_{G} comprise

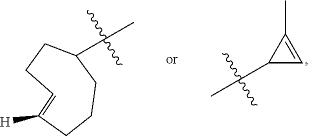

Y may comprise the

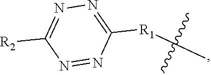

The R₂ and R₁ are as defined above in the present disclosure.

When there are multiple X_{F}, the above definitions regarding the X_{F} and the Y apply, if at least one X_{F} satisfies the above requirement.

For example, in the protein conjugate of hRs7-(Galβ1,4)GlcNAc-FD1, the Fuc* comprises a

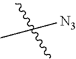 [SMILES: *N=[N+]=[N-]]

and a

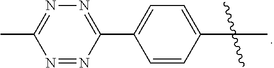 [SMILES: *c1ccc(-c2nnc(C)nn2)cc1]

The hRs7-(Galβ1,4)GlcNAc-FD1 (FIG. 5F) was then reacted with DBCO-PEG₄-GGFG-Dxd and TCO-PEG₄-vc-PAB-MMAE to obtain a protein conjugate comprising two Dxd and two MMAE linked to the Fuc* (MAR 2+2) (FIG. 7H and Example 42).

In another example, in the protein conjugate of trastuzumab-(GalNAzβ1,4)GlcNAc-FD1, the Fuc* comprises a

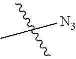 [SMILES: *N=[N+]=[N-]]

and a

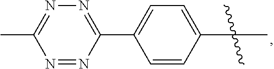 [SMILES: *c1ccc(-c2nnc(C)nn2)cc1]

the GalX comprises a

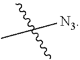 [SMILES: *N=[N+]=[N-]]

The trastuzumab-(GalNAzβ1,4)GlcNAc-FD1 (FIG. 5N) was then reacted with DBCO-PEG₄-vc-PAB-MMAF and TCO-PEG₄-vc-PAB-MMAE to obtain a protein conjugate comprising two MMAE and two MMAF linked to the Fuc* and two MMAF linked to the GalX (MAR 2+2+2) (FIG. 7G and Example 40).

FIG. 7 shows the MS analysis of some exemplary protein conjugates obtained by reacting the protein conjugate of the present disclosure with one or more Y-(L′)ₑ-P_{F}′.

FIG. 6 shows the molecular structure of some exemplary Y-(L′)ₑ-P_{F}′.

The protein conjugate of the present disclosure may have one or more of the following properties: having at least 2 MARs (active molecule to antibody ratio), and each of the MARs is about 2; having at least 2 MARs (active molecule to antibody ratio), and each of the MARs is about 4; being capable of binding to an antigen; being capable of binding to an antigen, with a similar binding affinity as the corresponding antibody; being stable in human plasma for at least 1 day; with the linkage between the Fuc* and the GlcNAc of Formula (I) being stable in human plasma for at least 1 day; being capable of participating in a bioorthogonal ligation reaction; being capable of inhibiting tumor growth and/or tumor cell proliferation.

In the present disclosure, the protein conjugate may have a first AM-to-antibody ratio (M₁AR), which is a ratio of the first active molecule (AM₁) in the Fuc* to the protein (e.g., the antibody). In the present disclosure, the protein conjugate may have a second AM-to-antibody ratio (M₂AR), which is a ratio of the second active molecule (AM₂) in the Fuc* to the protein (e.g., the antibody). In some cases, the protein conjugate may have a n^{th} AM-to-antibody ratio (MₙAR), which is a ratio of the n^{th} active molecule (AMₙ) in the Fuc* to the protein (e.g., the antibody), n is as defined in the present disclosure. In some cases, the protein conjugate may have a n+1^{th} AM-to-antibody ratio (Mₙ₊₁AR), which is a ratio of the n+1^{th} active molecule in the GalX to the protein (e.g., the antibody). When the active molecule is a pharmaceutically active molecule (e.g., comprising a drug, such as a cytotoxin or an agonist), the MAR may also be referred to as DAR (i.e., drug to antibody ratio).

### Preparation Method

In another aspect, the present disclosure provides a method for preparing a protein conjugate of the present application.

In one aspect, the present disclosure provides a method for preparing a protein conjugate, comprising step (a): contacting a fucose derivative donor Q-Fuc* with a protein comprising an oligosaccharide in the presence of a catalyst, wherein the oligosaccharide comprises Formula (VII): -GlcNAc(Fuc)_{b}-GalX (VII), to obtain a protein conjugate comprising the structure of Formula (I):

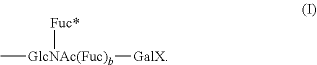

The GcNAc is directly orindirectly linkedto an amino acid ofthe protein. The GalX may be a galactose or a substituted galactose (i.e. the galactose may optionally be substituted). The Fuc is a fucose, and b is 0 or 1. Q is a diphosphate ribonucleotide, and the Fuc* is a fucose derivative comprising two or more active molecules AM. In some cases, b is 0. In some cases, b is 1.

The Q may be a uridine diphosphate (UDP), a guanosine diphosphate (GDP) or a cytidine diphosphate (CDP). In some cases, the Q-Fuc* is a GDP-Fuc*.

The catalyst may comprise a fucosyltransferase. For example, the fucosyltransferase may be an α-1,3-fucosyltransferase or a functional variants or fragments thereof: In some cases, the fucosyltransferase (such as the α-1,3-fucosyltransferase) may be derived from bacteria. For example, the fucosyltransferase (such as the α-1,3-fucosyltransferase) may be derived from Helicobacter pylori, such as Helicobacter pylori 26695.

For example, the fucosyltransferase may be the enzyme of GenBank Accession No. AAB81031.1, GenBank Accession No. AAD07447.1, GenBank Accession No. AAD07710.1, and/or their functional variants or fragments. A functional variant or fragment of the enzymes, may be its truncated form or a said enzyme with one or more (e.g., 1-2, 1-3, 1-4, 1-5, 1-6, 1-7. 1-8, 1-9, 1-10 or more) addition, deletion and/or substitutions.

For example, the fucosyltransferase may be the enzyme of GenBank Accession No. AAD07710.1, and/or a functional variant or fragment thereof: For example, the wild type fucosyltransferase of GenBank Accession No. AAD07710.1 comprises a catalytic domain, 10 Heptad Repeat Regions (HPRs) and a C-terminal tail. A functional variant or fragment of the wild type fucosyltransferase of GenBank Accession No. AAD07710.1, may be a truncated form thereof or a form with site-directed mutations. For example, a functional variant or fragment may comprise (or consist of) a catalytic domain and 1-10 HPRs (for example, 1, 2, 3, 4, 5, 6, 7, 8, 9 or 10 HPRs). In some embodiments, a functional variant or fragment thereof has an amino acid mutation at position C169 of the catalytic domain (e.g., with position C169 of SEQ ID NO: 14 being substituted by another amino acid). In some cases, the functional variant or fragment has the mutation C169S in its catalytic domain (such as in the catalytic domain of the fucosyltransferase of GenBank Accession No. AAD07710.1), accordingly, said variant may comprise a catalytic domain having an amino acid sequence as set forth in SEQ ID NO: 15.

In some cases, the fucosyltransferase may comprise a catalytic region and 1-10 Heptad Repeat Region (HPR) (for example, 1, 2, 3, 4, 5, 6, 7, 8, 9 or 10 HPRs), the catalytic region may be located N terminal to the HPRs. For example, the C terminus of the catalytic region may be ligated to the HPRs (e.g., the N terminus of the HPRs). The catalytic region may comprise an amino acid sequence as set forth in SEQ ID NO: 13, and X may be any amino acid residue. The catalytic region may comprise an amino acid sequence as set forth in SEQ ID NO: 14. The catalytic region may comprise an amino acid sequence as set forth in SEQ ID NO: 15. The HPR may comprise an amino acid sequence as set forth in SEQ ID NO: 12.

Comparing to the parent fucosyltransferase catalytic region (e.g., those as described above), the variant may comprise an amino acid sequence of catalytic region with a sequence identity of at least about 80% (e.g., at least about 82%, at least about 85%, at least about 88%, at least about 90%, at least about 92%, at least about 95%, at least about 98%, at least about 99% or more).

In some cases, the fucosyltransferase comprises an amino acid sequence as set forth in any of SEQ ID NOs: 16, 18, 20, 22 and 24.

In some cases, the catalyst of the present disclosure comprises a fucosyltransferase of present disclosure and a fusion tag (such as a His tag).

For exapmle, the catalyst may comprise an amino acid sequence as set forth in any of SEQ ID NO: 17, 19, 21, 23 and 25.

The Fuc* may comprise the structure of Formula (II):

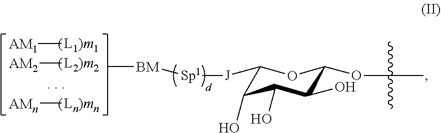

wherein: J is a jointer, and the J may be directly linked to the

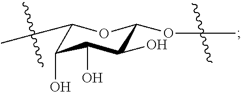

Sp¹ is a spacer moiety, d is 0 or 1; BM is a branching moiety; L₁ to Lₙ each independently is a linker, m₁ to mₙ each independently is 0 or 1; AM₁ to AMₙ each independently is an active molecule; and n is an integer from 2-10. The various AMs (i.e., AM₁, AM₂ . . . , AMₙ) may be the same or may be different from each other. The various linkers (i.e., L₁, L₂, . . . , Lₙ) may be the same or may be different from each other.

The GalX may be linked to the GlcNAc through a β1,4 linkage. For example, the C1 position of the GalX is linked to the C4 position of the GlcNAc through a —O—. The Fuc may be linked to the GlcNAc through an α1,6 linkage. For example, the C1 position of the Fuc is linked to the C6 position of the GlcNAc through a —O—.

The Fuc* may be linked to the GlcNAc through an α1,3 linkage. For example, the C1 position of the Fuc* is linked to the C3 position of the GlcNAc through a —O—.

The branching moiety BM may comprise

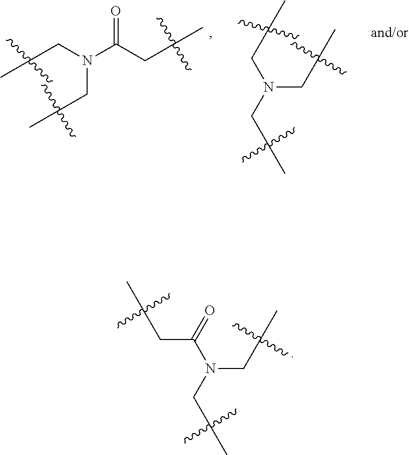 [SMILES: *CC(=O)N(C*)C* ; *CN(C*)C* ; *CC(=O)N(C*)C*]

For example, BM may comprise one or more structures selected from

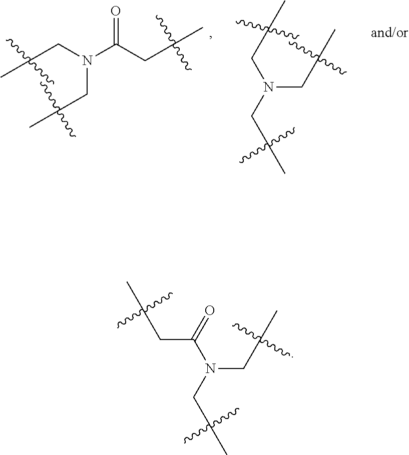 [SMILES: *CC(=O)N(C*)C* ; *CN(C*)C* ; *CC(=O)N(C*)C*]

The right side of the structure of BM may be linked (e.g., directly linked) to the Sp¹ or J. For example, when d is 0, the right side of the structure of BM is linked (e.g., directly linked) to J. When d is 1, the right side of the structure of BM is linked (e.g., directly linked) to Sp¹ and Sp¹ is in turn linked (e.g., directly linked) to J.

In some cases, n may be 2, and the Fuc* may comprise the structure of Formula (III):

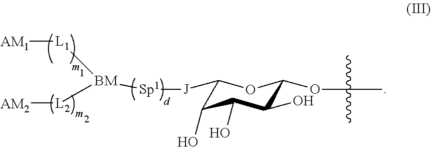

For example, BM is selected from the group consisting of:

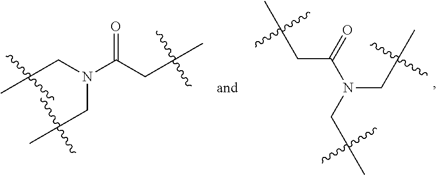 [SMILES: *CC(=O)N(C*)C* ; *CC(=O)N(C*)C*]

wherein the right side of the structure is directly linked to the Sp¹ or J.

The jointer J may have a structure of

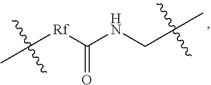

wherein R_{f} is —CH₂—, —NH— or —O—. In some cases, the jointer J is

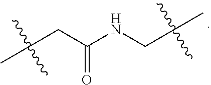 [SMILES: *CNC(=O)C*]

The right side of the structure of J may be linked (e.g., directly linked) to the left side of

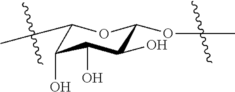

### of Formula (II).

In the present disclosure. the Q-Fuc* comprising different jointers may have different conversion efficiency on the antibodies comprising a Fc fragment or Fc-fusion proteins by using α1,3 fucosyltransferases. For example, the Q-Fuc* comprising a jointer of

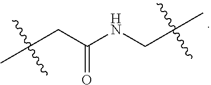 [SMILES: *CNC(=O)C*]

would have high conversion efficiency. For example, the Q-Fuc* comprising a jointer of

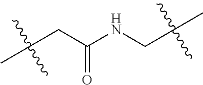 [SMILES: *CNC(=O)C*]

would have significant higher conversion efficiency than the Q-Fuc* comprising a jointer of

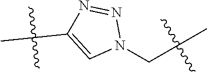 [SMILES: *Cn1cc(*)nn1]

on the antibodies comprising a Fc fragment or Fc-fusion proteins by using a Helicobacter pylori α1,3 fucosyltransferase. For example, example 32 shows the comparison of the conversion efficiency of Helicobacter pylori α1,3 fucosyltrasferase towards GDP-fucose derivatives with different jointers on antibody-G₂F, antibody-(Galβ1,4)GlcNAc, antibody-(Fucα1,6)(Galβ1,4)GlcNAc and antibody-(GalNAzβ1,4)GlcNAc, respectively.

Sp¹ may be a structure selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs². For example, each of the alkylene, cycloalkylene, alkenylene, cycloalkenylene, alkynylene, cycloalkynylene, (hetero)arylene, (hetero)arylalkylene and alkyl(hetero)arylene may independently be substituted with one or more Rs¹. In some cases, one or more Rs² may be inserted in the alkylene, cycloalkylene, alkenylene, cycloalkenylene, alkynylene, cycloalkynylene, (hetero)arylene, (hetero)arylalkylene and/or alkyl(hetero)arylene. For example, an alkylene may be inserted by one or more —O— to become a -PEG-.

Each Rs¹ may independently be selected from the group consisting of halogen, —OH, —NH₂ and —COOH.

Each Rs² may independently be selected from the group consisting of —O—, —S—,

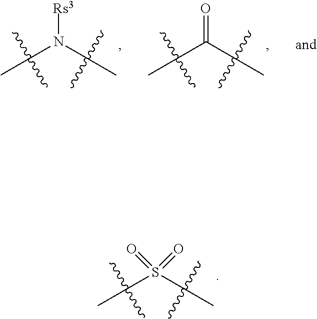

Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the Sp¹ may be selected from the group consisting of:

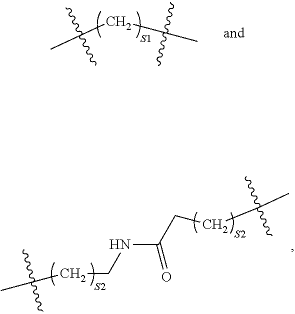

S1 may be an integer from 1-50 (for example, 1-40, 1-30, 1-20, 1-15, 1-14, 1-13, 1-12, 1-11, 1-10, 1-9, 1-8, 1-7, 1-6, 1-5, 1-4, 1-3, 1-2, or 1), each S2 may independently be an integer from 0-50 (for example, 0-40, 0-30, 0-20, 0-15, 0-14, 0-13, 0-12, 0-11, 0-10, 0-9, 0-8, 0-7, 0-6, 0-5, 0-4, 0-3, 0-2, 0-1 or 0). Each said —CH₂—(—CH₂— in the parentheses) may independently be replaced by a —O—, with the proviso that two or more consecutive —CH₂— are not simultaneously replaced by —O—. Accordingly, when one —CH₂— is replaced by a —O—, its immediate neighboring —CH₂— to the left and to the right may not be replaced by —O—. The right side of the structure of the Sp¹ may be linked to the J and the left side of the structure of the Sp¹ may be linked to the BM.

In some cases, the Sp¹ may be

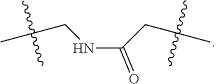 [SMILES: *CNC(=O)C*]

In some cases, the Sp¹ may be

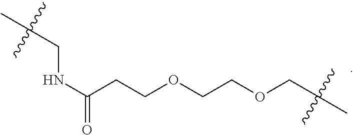 [SMILES: *CNC(=O)CCOCCOC*]

The right side of the structure of the Sp¹ may be linked to the J and the left side of the structure of the Sp¹ may be linked to the BM.

In some cases, d is 0 (meaning that the Sp¹ is absent). The BM is directly linked to the J. For example, the GDP-FD4, GDP-FD5 and GDP-FD6 in FIG. 2 comprise a J of

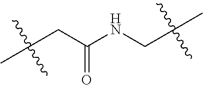 [SMILES: *CNC(=O)C*]

### and a BM of:

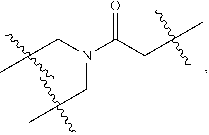 [SMILES: *CC(=O)N(C*)C*]

and the BM is directly linked to the J.

Each of L₁ to Lₙ may independently be a linker of Formula (IV): (CL)_{y}-(FL)ₓ (IV). The various L (i.e., L₁, L₂ . . . , Lₙ) may be the same or may be different from each other. FL is a spacer moiety, x is 0 or 1, CL is a cleavable linker, y is 0 or 1, the right side of Formula (IV) is linked to said BM and the left side of Formula (IV) is linked to said AM. For example, the FL side is linked to the BM and the CL side is linked to the AM.

The FL may be a spacer moiety selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs². For example, one or more of the alkylene, cycloalkylene, alkenylene, cycloalkenylene, alkynylene, cycloalkynylene, (hetero)arylene, (hetero)arylalkylene and alkyl(hetero)arylene may be substituted by one or more Rs¹. In some cases, one or more Rs² may be inserted in the alkylene, cycloalkylene, alkenylene, cycloalkenylene, alkynylene, cycloalkynylene, (hetero)arylene, (hetero)arylalkylene and/or alkyl(hetero)arylene. For example, an alkylene may be inserted by one or more —O— to become a -PEG-.

Each Rs¹ may independently be selected from the group consisting of halogen, —OH, —NH₂ and —COOH, and each Rs² may independently be selected from the group consisting of —O—, —S—,

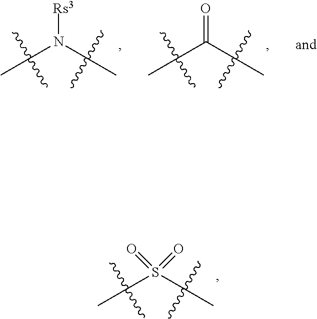

Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the FL is a spacer moiety selected from the group consisting of:

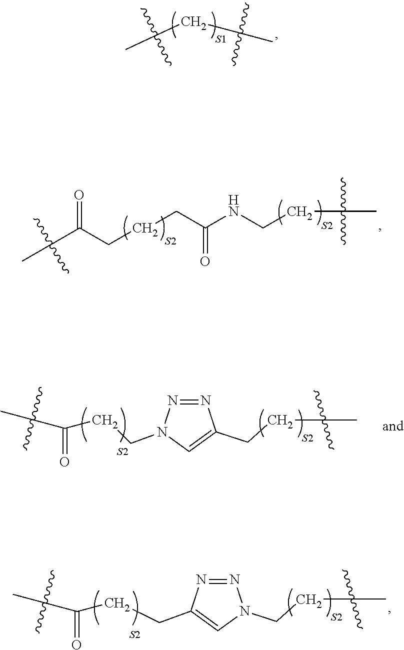

wherein said S1 may independently be an integer from 1-50 (for example, 1-40, 1-30, 1-20, 1-15, 1-14, 1-13, 1-12, 1-11, 1-10, 1-9, 1-8, 1-7, 1-6, 1-5, 1-4, 1-3, 1-2 or 1), each S2 may independently be an integer from 0-50 (for example, 0-40, 0-30, 0-20, 0-15, 0-14, 0-13, 0-12, 0-11, 0-10, 0-9, 0-8, 0-7, 0-6, 0-5, 0-4, 0-3, 0-2, 0-1 or 0). Each said —CH₂—(—CH₂— in the parentheses) may independently be replaced by a —O—, with the proviso that two or more consecutive —CH₂— are not simultaneously replaced by —O—. Accordingly, when one —CH₂— is replaced by a —O—, its immediate neighboring —CH₂— to the left and to the right may not be replaced by —O—. The right side of the structure of the FL may be linked to the BM, and the left side of the structure of the FL may be linked to the CL or the AM.

In some cases, the FL is a spacer moiety selected from the group consisting of:

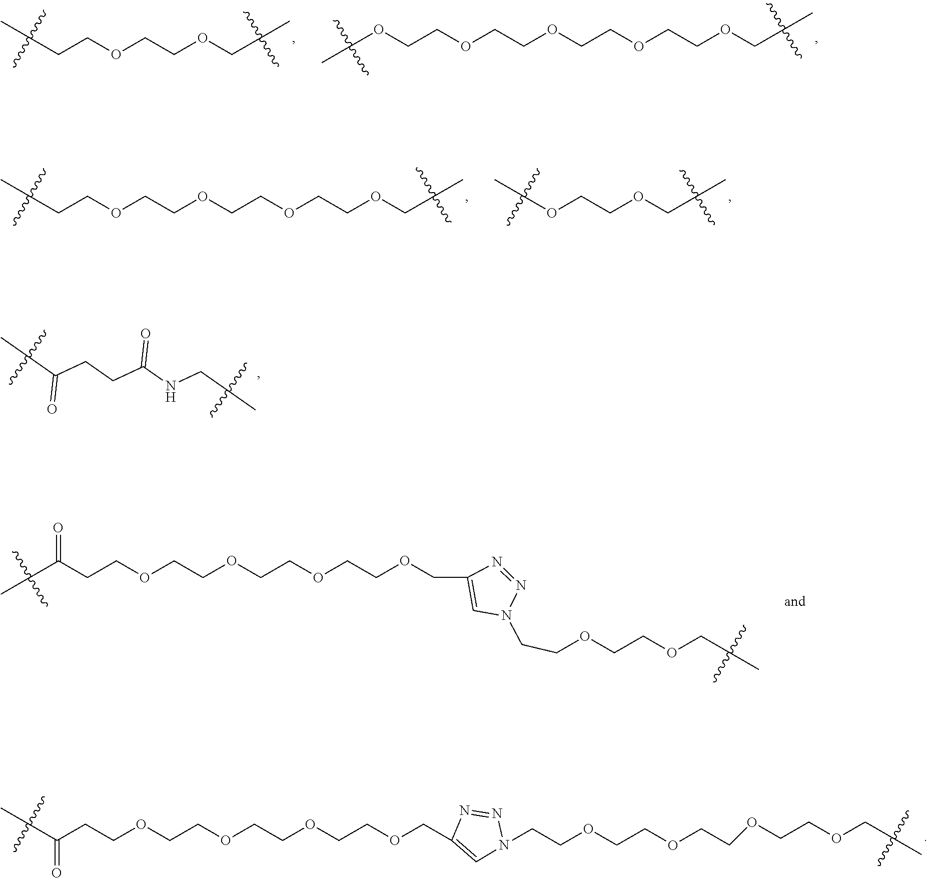

The right side of the structure of the FL may be linked to the BM, and the left side of the structure of the FL may be linked to the CL or the AM.

In some cases (e.g., for some of the L), x is 0 (meaning that FL is absent), and y is 1, the CL is linked (e.g., directly linked) to the corresponding AM and the BM. For example, the right side of CL is linked to the BM and the left side of the CL is linked to the AM. In some cases (e.g., for some of the L), y is 0 (meaning that CL is absent), and x is 1, the FL is linked (e.g., directly linked) to the corresponding AM and the BM. For example, the right side of FL is linked to the BM and the left side of the FL is linked to the AM. In some cases, both x and y are 0, meaning that the specific L is absent, and the corresponding AM may be directly linked to the BM.

In some cases, both x and y are 1, the FL is (directly) linked to the CL and the BM, and the CL is in turn (directly) linked to the corresponding AM. For example, the right side of FL is linked to the BM and the left side of the CL is linked to the AM. The CL may be an acid-labile linker, a redox-active linker, a photo-active linker and/or a proteolytically cleavable linker. In some cases, the CL may be a vc-PAB-linker and/or a GGFG-linker.

In the present disclosure, each of AM₁ to AMₙ may independently be a chemically active molecule, an enzymatically active molecule, a biologically active molecule, and/or a pharmaceutically active molecule. For example, one AM may be the same as another AM, or different AMs may be different from each other. Each AM may independently be a chemically active molecule, an enzymatically active molecule, a biologically active molecule, or a pharmaceutically active molecule.

In some cases, the AM₁ to AMₙ independently comprises a chemically or enzymatically active molecule X_{F}. For example, AM₁ to AMₙ may comprise one or more X_{F}. The chemically or enzymatically active molecule X_{F} may comprise a functional moiety capable of participating in a ligation reaction. For example, the X_{F} may comprise a functional moiety capable of participating in a bioorthogonal ligation reaction.

In some cases, the X_{F} may comprise a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof:

In some cases, the X_{F} may comprise a functional moiety selected from the group consisting of

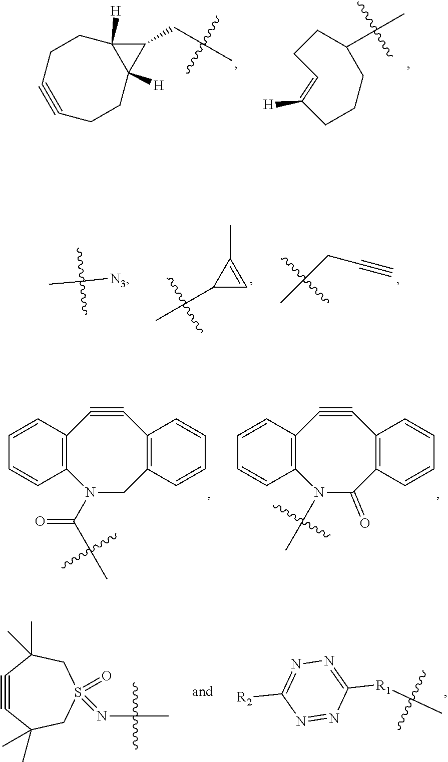

wherein R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group.

In some cases, the X_{F} comprises a functional moiety selected from the group consisting of:

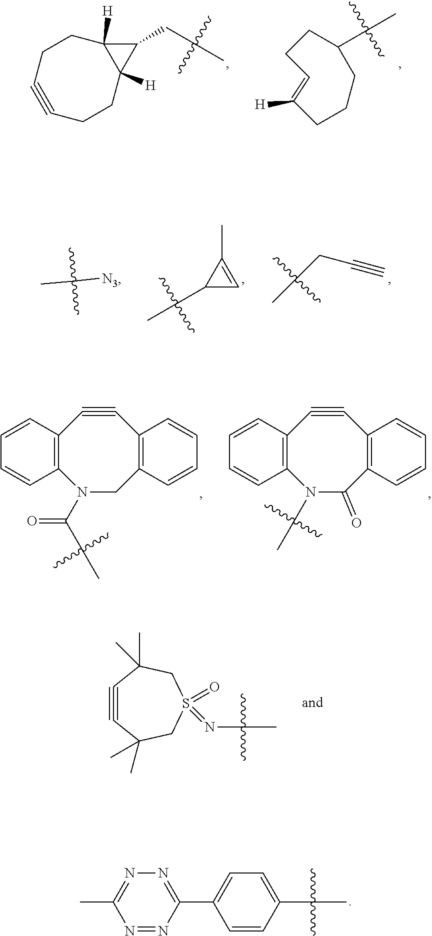

In some cases, the AM₁ to AMₙ independently comprises a biologically active molecule and/or pharmaceutically active molecule P_{F}. For example, AM₁ to AMₙ may comprise one or more P_{F}. The P_{F} may comprise a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof:

In some cases, the P_{F} is a pharmaceutically active molecule.

For example, the P_{F} may comprise a cytotoxin, an agonist, an antagonist, an antiviral agent, an antibacterial agent, an oligonucleotide, a polypeptide or any combination thereof: In some cases, the P_{F} comprises a cytotoxin or an agonist (such as a sting agonist, or a toll like receptor (such as TLR7/8) agonist).

In some cases, the P_{F} comprises a DNA or RNA damaging agent, an RNA polymerase inhibitor, a topoisomerase inhibitor and/or a microtubule inhibitor.

In some cases, the P_{F} comprises a pyrrolobenzodiazepine, an auristatin, a maytansinoids, a duocarmycin, a tubulysin, an enediyene, a doxorubicin, a pyrrole-based kinesin spindle protein inhibitor, a calicheamicin, an amanitin, a camptothecin and/or derivatives thereof:

In some cases, the P_{F} comprises an MMAE, a DXd, T785 and/or their derivatives thereof:

In the present disclosure, the GalX may be a galactose, or a substituted galactose. In some cases, the GalX is a monosaccharide (e.g., after substitution, the substituted GalX is still a monosaccharide, for example, the substituted GalX only comprises one monosaccharide unit, for example, GalNAz is a monosaccharide).

In some cases, the GalX may be a galactose.

In some cases, the GalX may be a substituted galactose, and the hydroxyl group at one or more positions selected from the C2 position, the C3 position, the C4 position and the C6 position of the galactose is substituted. For example, the GalX may be a substituted galactose, wherein the hydroxyl group at the C2 position of the galactose is substituted.

In some cases, the GalX may be a galactose substituted by

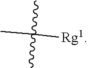

The Rg¹ may be selected from the group consisting of hydrogen, halogen, —NH₂, —SH, —N₃, —COOH, —CN, C₁-C₂₄ alkyl, C₃-C₂₄ cycloalkyl, C₂-C₂₄ alkenyl, C₅-C₂₄ cycloalkenyl, C₂-C₂₄ alkynyl, C₆-C₂₄ cycloalkynyl, C₂-C₂₄ (hetero)aryl, C₃-C₂₄ alkyl(hetero)aryl, C₃-C₂₄ (hetero)arylalkyl and any combination thereof: Each of the alkyl, cycloalkyl, alkenyl, cycloalkenyl, alkynyl, cycloalkynyl, (hetero)aryl, alkyl(hetero)aryl and (hetero)arylalkyl may independently be substituted by one or more Rs⁴ and/or may independently be interrupted by one or more Rs⁵. For example, one or more of the alkyl, cycloalkyl, alkenyl, cycloalkenyl, alkynyl, cycloalkynyl, (hetero)aryl, alkyl(hetero)aryl and (hetero)arylalkyl may independently be substituted by one or more Rs⁴. In some cases, one or more Rs⁵ may be inserted in the alkyl, cycloalkyl, alkenyl, cycloalkenyl, alkynyl, cycloalkynyl, (hetero)aryl, alkyl(hetero)aryl or (hetero)arylalkyl. For example, the alkyl may be inserted by one or more —O— to become a -PEG.

Each Rs⁴ may independently be selected from the group consisting of halogen, —OH, —NH₂, —SH, —N₃, —COOH and —CN. Each Rs⁵ may independently be selected from the group consisting of —O—, —S—,

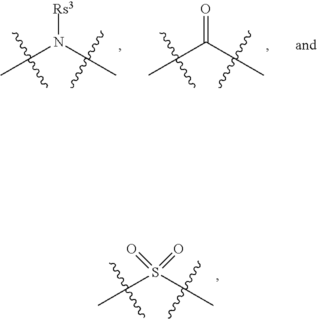

and Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the GalX may be a galactose substituted by

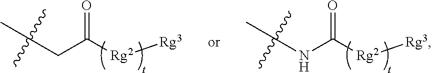

wherein t is 0 or 1, Rg² is selected from the group consisting of C₁-C₂₄ alkylene, C₃-C₂₄ cycloalkylene, C₂-C₂₄ alkenylene, C₅-C₂₄ cycloalkenylene, C₂-C₂₄ alkynylene, C₆-C₂₄ cycloalkynylene, C₂-C₂₄ (hetero)arylene, C₃-C₂₄ alkyl(hetero)arylene and C₃-C₂₄ (hetero)arylalkylene. Each of the alkylene, cycloalkylene, alkenylene, cycloalkenylene, alkynylene, cycloalkynylene, (hetero)arylene, alkyl(hetero)arylene and (hetero)arylalkylene may independently be substituted by one or more Rs⁴ and/or may independently be interrupted by one or more Rs⁵. For example, one or more of the alkylene, cycloalkylene, alkenylene, cycloalkenylene, alkynylene, cycloalkynylene, (hetero)arylene, alkyl(hetero)arylene and (hetero)arylalkylene may independently be substituted by one or more Rs⁴. In some cases, one or more Rs¹ may be inserted in the alkylene, cycloalkylene, alkenylene, cycloalkenylene, alkynylene, cycloalkynylene, (hetero)arylene, (hetero)arylalkylene or alkyl(hetero)arylene. For example, alkylene may be inserted by one or more —O— to become a -PEG-.

Rg³ may be selected from the group consisting of hydrogen, halogen, —OH, —NH₂, —SH, —N₃, —COOH, —CN, C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl, C₂-C₂₄ (hetero)aryl and any combination thereof, wherein each of the C₁-C₂₄ alkyl, C₃-C₂₄ cycloalkyl, C₂-C₂₄ alkyne, C₅-C₂₄ cycloalkyne, C₂-C₂₄ alkynyl, C₈-C₂₄ cycloalkynyl and C₂-C₂₄ (hetero)aryl may independently be substituted by one or more Rs⁴.

Each Rs⁴ may independently be selected from the group consisting of halogen, —OH, —NH₂, —SH, —N₃, —COOH and —CN.

Each Rs⁵ may independently be selected from the group consisting of —O—, —S—,

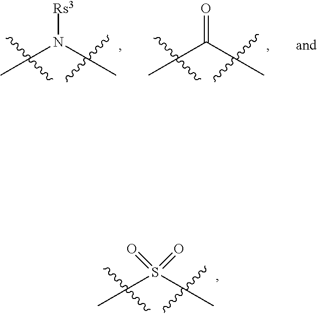

wherein Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the GalX may comprise a chemically and/or enzymatically active molecule X_{G}. The X_{G} may comprise a functional moiety capable of participating in a ligation reaction. The X_{G} may comprise a functional moiety capable of participating in a bioorthogonal ligation reaction. The X_{G} may comprise a functional moiety selected from the group consisting of azido, terminal alkynyl, cyclic alkynyl, tetrazinyl, 1,2,4-trazinyl, terminal alkenyl, cyclic alkenyl, ketone, aldehyde, hydroxyl amino, sulfhydryl, N-maleimide and functional derivatives thereof. For example, the X_{G} may comprise a

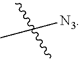 [SMILES: *N=[N+]=[N-]]

In some cases, when one or more of the AMs comprises a X_{F}, and the GalX comprises a X_{G}, the X_{G} does not substantially react with any X_{F}. For example, the X_{G} may comprise a

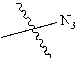 [SMILES: *N=[N+]=[N-]]

and the X_{F} may comprise a

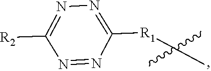

R₁ and R₂ are as defined in the present disclosure. In some cases, the X_{G} and the X_{F} may comprise the same functional moiety. For example, the X_{G} may comprise a

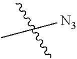 [SMILES: *N=[N+]=[N-]]

and the X_{F} may comprise a

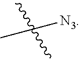 [SMILES: *N=[N+]=[N-]]

In some cases, the GalX is selected from the group consisting of

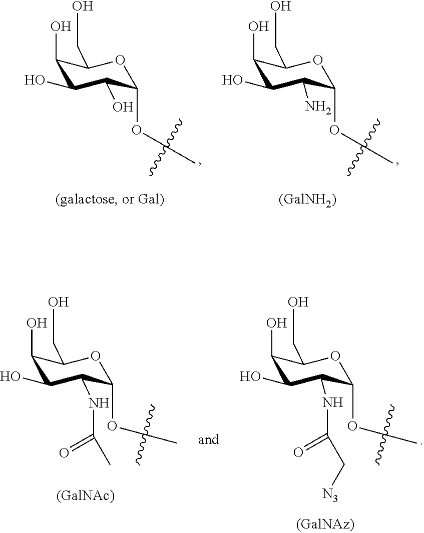

The protein may comprise an antigen binding fragment and/or an Fc fragment. The Fc fragment may be an IgG Fc fragment. In some cases, the oligosaccharide may be linked to the Fc fragment. For example, the oligosaccharide may be linked to the CH₂ domain of the Fc fragment.

The oligosaccharide of the protein may be an N-linked oligosaccharide. The oligosaccharide may be linked to an Asparagine (Asn) residue of the protein. For example, the GlcNAc of Formula (VII) may be directly linked to an Asn residue of the protein.

In some cases, the oligosaccharide may be linked to the Asn297 of the Fc fragment, numbered according to the Kabat numbering system.

In some cases, the GlcNAc of Formula (VII) may be linked to a saccharide of the oligosaccharide.

For example, the GlcNAc of Formula (VII) may be linked to a mannose of the oligosaccharide, and b may be 0.

The protein of the present disclosure may be an antibody. For example, the protein of the present disclosure may be a monoclonal antibody. In some cases, the protein of the present disclosure may be an IgG antibody. In some cases, the protein of the present disclosure may be a humanized antibody.

For example, the protein of the present disclosure may be a nanobody, ScFv, Fab, F(ab)₂, F(ab′) and/or F(ab′)₂.

In some cases, the protein may comprise a Fc fragment and an antigen binding fragment. The protein may be an antibody or a fragment thereof, as defined in the present disclosure. For example, the antibody may recognize a target antigen. For example, the target antigen may be Her2, Her3, Trop2, EGFR, BCMA, Nectin-4, MUC1, c-Met, PSMA, GD2, GPC3, CEA, CD20, ErbB3, ErbB4, PD-L1 and/or EpCAM. For example, the target antigen may be Trop2 or Her2.

In the present disclosure, the protein may be an antibody or a fragment thereof. For example, the antibody could be but not limited to trastuzumab, bevacizumab, rituximab, durvalumab, pertuzumab, raxibacumab, dinutuximab, ixekizumab, labetuzumab, odesivimab. risankizumab, dinutuximab, adalimumab, cetuximab, daratumumab, tocilizumab, hRS7 and etc. For example, the antibody may be trastuzumab or hRS7. The sequences of the antibodies or the fragment thereof are as defined in the present disclosure.

In some cases, the protein is a Fc-fusion protein. The Fc-fusion protein may comprise a Fc fragment and a biologically active protein or polypeptide. For example, the biologically active protein or polypeptide may be therapeutically effective. For example, the biologically active protein may be derived from a non-immunoglobulin protein. For example, the biologically active protein may be a cytokine, a complement, and/or an antigen, or a fragment thereof:

In some cases, n is 2, the Fuc* comprises AM₁ and AM₂, both the AM₁ and the AM₂ comprises a X_{F}, the X_{F} of the AM₁ and the X_{F} of the AM₂ may be identical or different.

In some cases, n is 2, the Fuc* comprises AM₁ and AM₂, both the AM₁ and the AM₂ comprises a X_{F}, the X_{F} of the AM₁ and the X_{F} of the AM₂ is independently selected from the group consisting of:

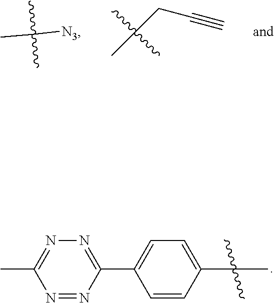

However, the X_{F} of AM₁ may not react bioorthogonally with the X_{F} of AM₂. For example, when the X_{F} of the AM₁ is

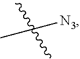 [SMILES: *N=[N+]=[N-]]

the X_{F} of the AM₂ shall not be

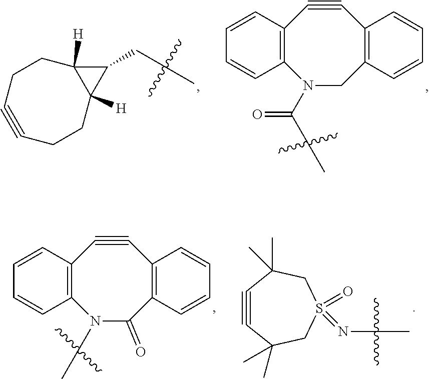

In another example, when the X_{F} of the AM₁

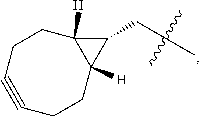 [SMILES: *C[C@H]1[C@H]2CCC#CCC[C@@H]12]

the X_{F} Of the AM₂ shall not be

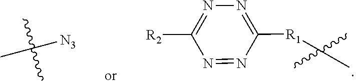

In another example, when the X_{F} of the AM₁ is

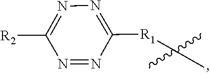

the X_{F} of the AM₂ shall not be

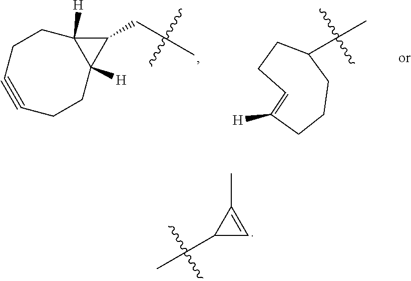

In some cases, n is 2, the Fuc* comprises AM₁ and AM₂, the AM₁ may comprise a X_{F} and the AM₂ may comprise a P_{F}; or the AM₁ may comprise a P_{F} and the AM₂ may comprise a X_{F}.

In some cases, n is 2, the Fuc* comprises AM₁ and AM₂, both the AM₁ and the AM₂ comprises a P_{F}, the P_{F} of AM₁ and the P_{F} of AM₂ are identical or different.

According to any aspect of the present disclosure, the Q-Fuc* may have a structure selected from the followings:

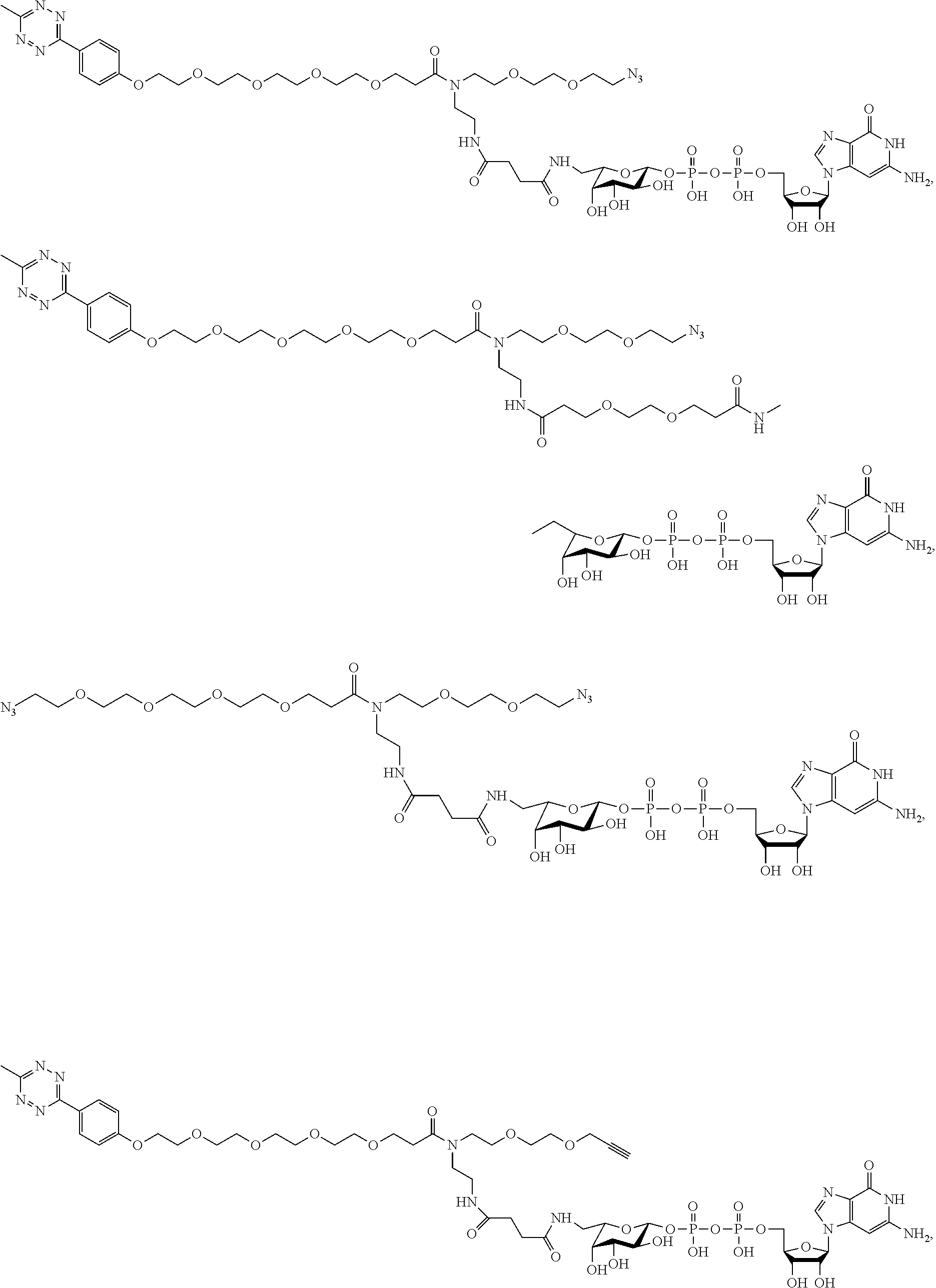

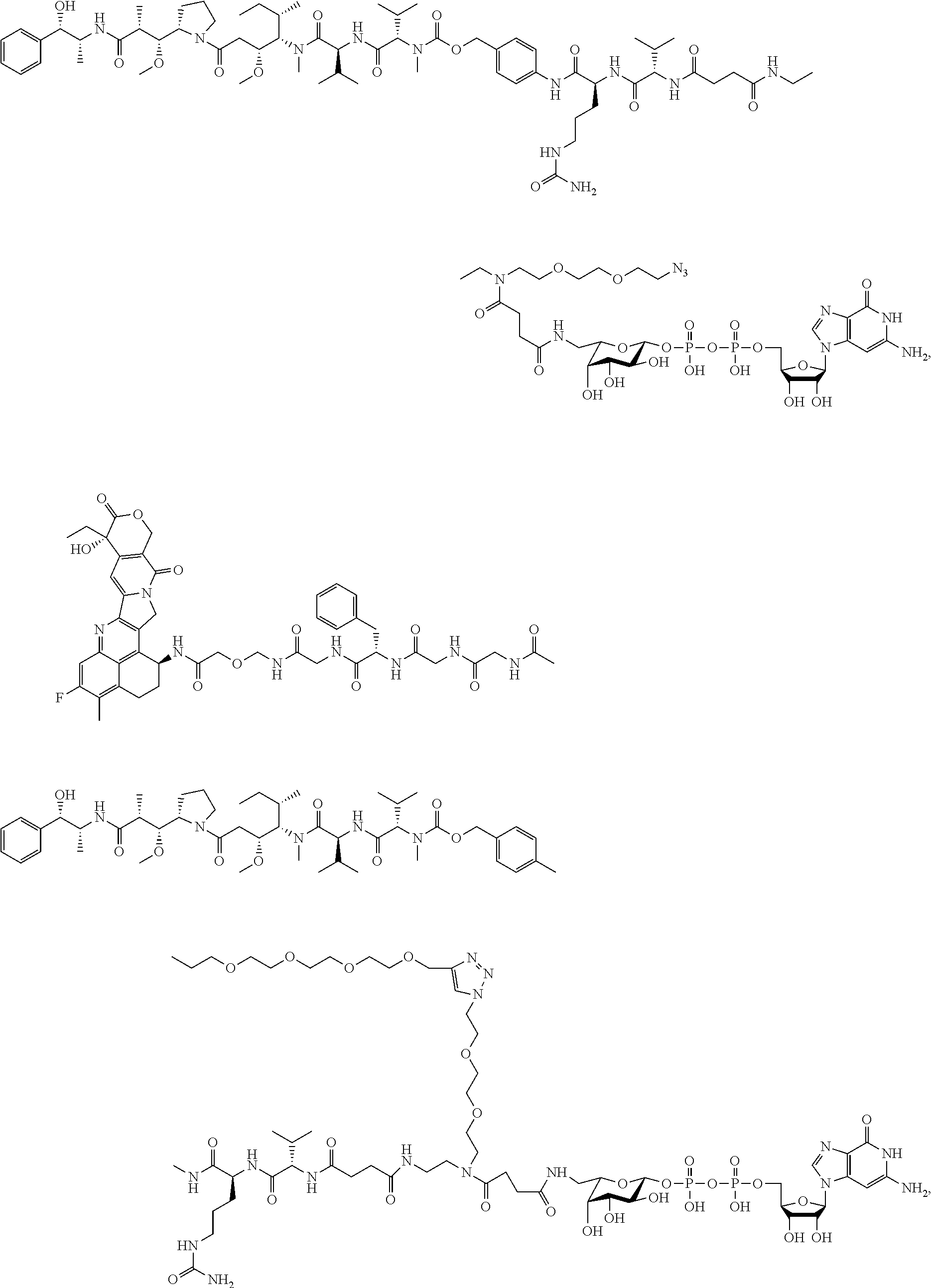

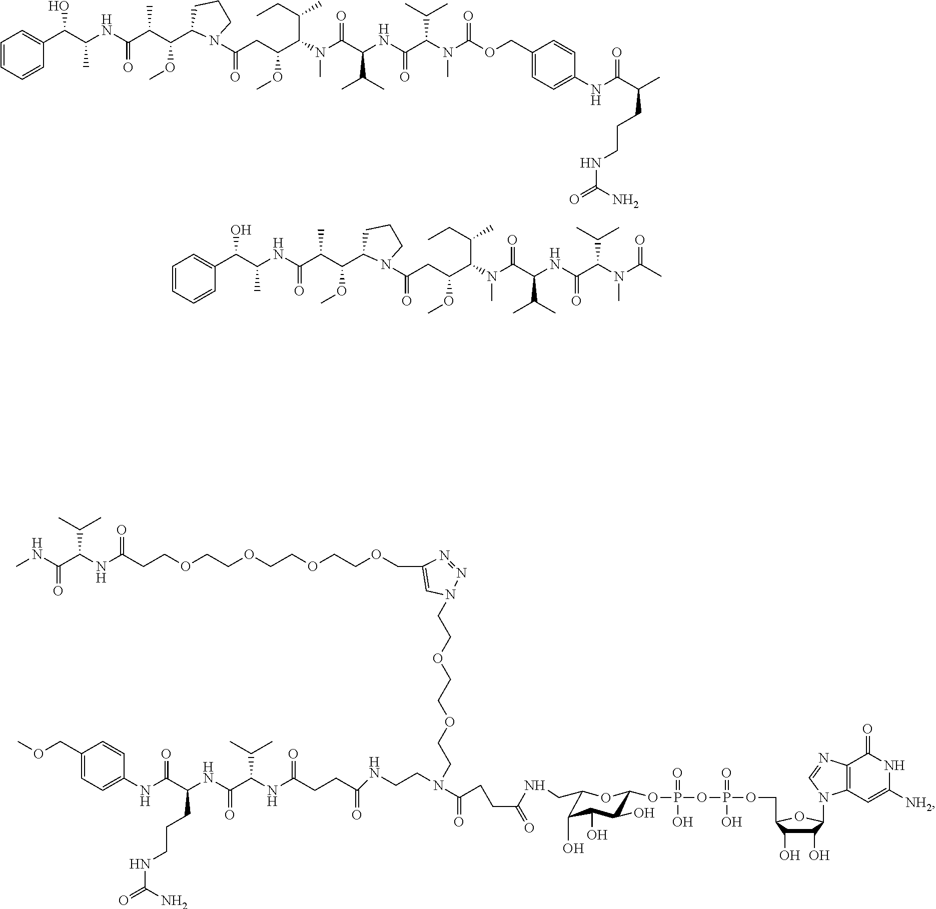

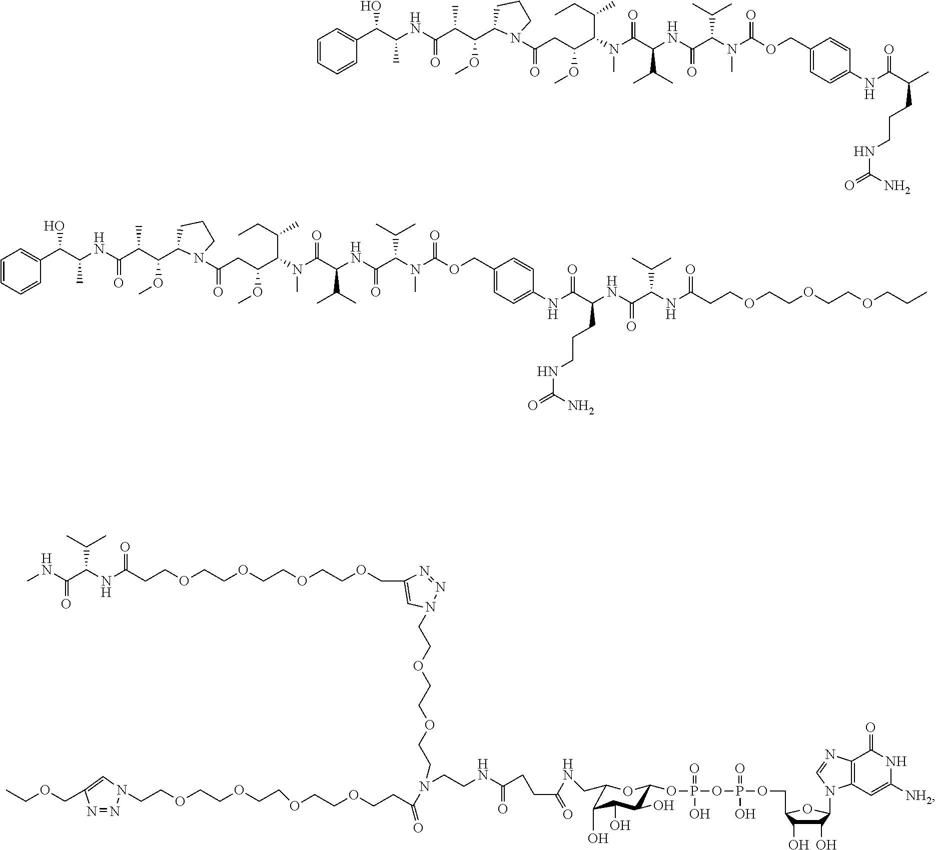

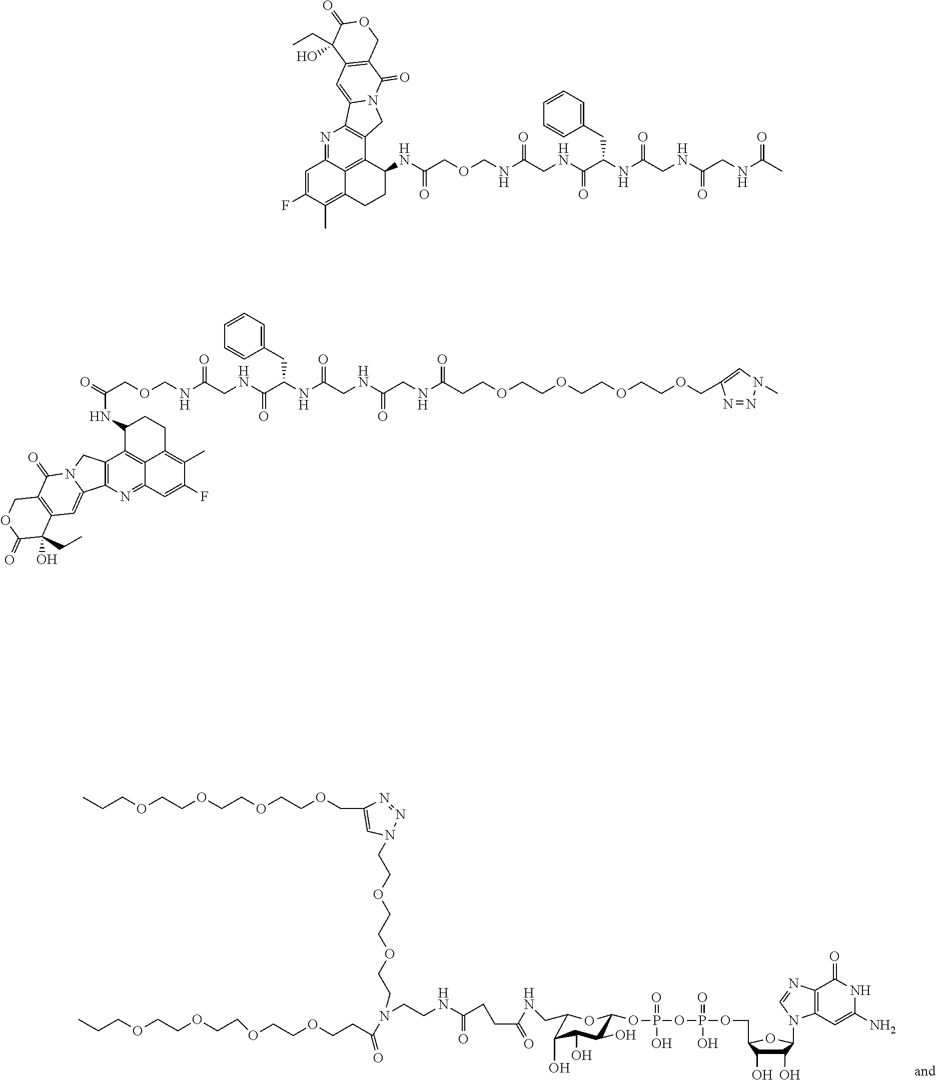

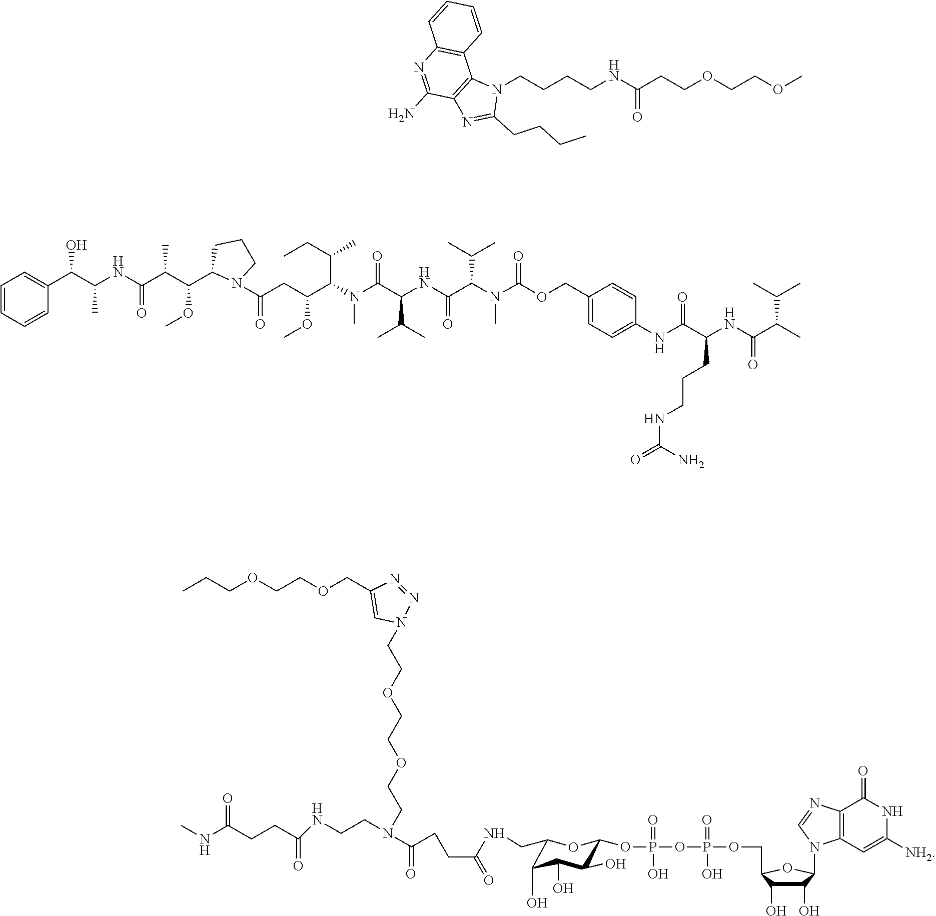

In the method of the present disclosure, the protein may comprise 1-20 (e.g., 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, or 1-20) of the structure of -GlcNAc (Fuc)_{b}-GalX (VII). In some cases, the protein comprises 2 or 4 of the structure of -GlcNAc (Fuc)_{b}-GalX (VII).

In some cases, the protein comprises 2 of the structure of -GlcNAc (Fuc)_{b}-GalX (VII). For example, the protein comprising the oligosaccharide may comprise a structure of Formula (VIII)

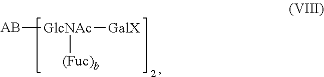

wherein AB is an antibody comprising a Fc fragment or a Fc-fusion protein, the GlcNAc is directly linked to an Asn of the Fc fragment of the AB, the Fuc is linked to the GlcNAc through an α1,6 linkage, the GalX is linked to the GlcNAc through a β1,4 linkage, and b is 0 or 1. In some cases, the GlcNAc is directly linked to an N297 of the Fc fragment of the AB. In some cases, b is 0. For example, when b is 0, the structure of Formula (VIII)

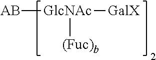

may be

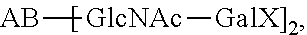

In some cases, b is 1.

In some cases, the method further comprises the steps of: i) modifying a glycosylated antibody comprising the Fc fragment or the Fc-fusion protein comprising an oligosaccharide with an endoglycosidase to obtain a modified protein; and ii) contacting the modified protein of i) with a UDP-GalX in the presence of a catalyst to obtain the protein comprising the structure of Formula (VIII), the b in Formula (VIII) may be 0 or 1. In some cases, b is 0. In some cases, b is 1. In some case, for example, when the glycosylated antibody comprises a core α-1,6 fucose, then b is 1. In some case, for example, when the glycosylated antibody doesn't comprise a core α-1,6 fucose, then b is 0.

In some cases, the method further comprises the steps of: i) modifying a glycosylated antibody comprising an Fc fragment or the Fc-fusion protein with an endoglycosidase and an α1,6 fucosidase to obtain a modified protein; and ii) contacting the modified protein of i) with a UDP-GalX in the presence of a catalyst to obtain the protein comprising the structure of Formula (VIII), the b in Formula (VIII) may be 0.

The endoglycosidase may be an Endo S, Endo S2, Endo A, Endo F, Endo M, Endo D, Endo H or their functional mutants or variants, or any combination thereof: For example, the endoglycosidase may be an EndoS. For example, the endoglycosidase may have an amino acid sequence as set forth in SEQ ID NO: 3 or 4, or a functional variant or fragment thereof:

The α1,6 fucosidase may be a BfFucH, a fucosidase O, an Alfe, a BKF, a fucosidase O or their functional mutants or variants, or any combination thereof. For example, the α1,6 fucosidase may be Alfc. For example, the α1,6 fucosidase may be an enzyme comprising an amino acid sequence as set forth in any one of SEQ ID NO: 5-6, or a functional variant or fragment thereof:

The catalyst employed in the step ii) may be a β1,4-galactosyltransferase, or a functional variant or fragment thereof: In some embodiments, the catalyst is a human β1,4-galactosyltransferase, a bovine β1,4-galactosyltransferase, or a functional variant or fragment thereof: In some embodiments, the catalyst comprises a catalytic region of bovine β(1,4)-GalT1 with an mutation of Y289L, Y289N, Y289I, Y289F, Y289M, Y289V, Y289G, Y289I or Y289A, or a catalytic region of human β(1,4)-GalT1 with an mutation of Y285L, Y285N, Y285I, Y285F, Y285M, Y285V, Y285G, Y285I or Y285A. In some embodiments, the catalyst comprises an amino acid sequence as set forth in any one of SEQ ID NO: 1-2.

In some cases, the protein comprises 4 of the structure of -GlcNAc(Fuc)_{b}-GalX (VII).

For example, in some cases, the protein comprises the structure of Formula (IX)

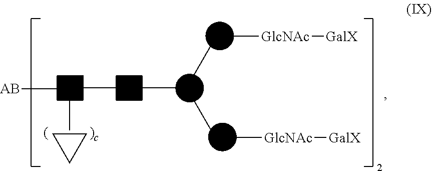

wherein AB is an antibody comprising a Fc fragment or a Fc-fusion protein, [symbol] is a GlcNAc, [symbol] is a mannose, [symbol] is a fucose linked to the [symbol] through a α1,6 linkage, c is 0 or 1; the oligosaccharide is linked to an Asn of the Fc fragment of the AB through the [symbol] , and the GalX is linked to the GlcNAc through a β1,4 linkage.

In some cases, the method further comprises contacting the antibody comprising an Fc fragment or the Fc-fusion protein having a glycoform of G₀(F)_{0,1}, G₁(F)_{0,1} and/or G₂(F)_{0,1} with a UDP-GalX in the presence of a catalyst, to obtain the protein comprising the structure of Formula (IX). In some cases, the method further comprises contacting the antibody comprising an Fc fragment or the Fc-fusion protein having a glycoform of G₀(F)_{0,1} with a UDP-GalX in the presence of a catalyst, to obtain the protein comprising the structure of Formula (IX). The catalyst may be a β1,4-galactosyltransferase, or a functional variant or fragment thereof: In some embodiments, the catalyst is a human β1,4-galactosyltransferase, a bovine β1,4-galactosyltransferase, or a functional variant or fragment thereof: In some embodiments, the catalyst comprises a catalytic region of bovine β(1,4)-GalT1 with an mutation of Y289L, Y289N, Y289I, Y289F, Y289M, Y289V, Y289G, Y289I or Y289A, or a catalytic region of human β(1,4)-GalT1 with an mutation of Y285L, Y285N, Y285I, Y285F, Y285M, Y285V, Y285G, Y285I or Y285A. In some embodiments, the catalyst comprises an amino acid sequence as set forth in any one of SEQ ID NO: 1-2.

In the present disclosure, in some embodiments, a protein comprising a -GlcNAc(Fuc)_{b}-GalX linked directly to the Asn of the Fc fragment may have much higher conversion efficiency compared to that comprising a -GlcNAc-GalX linked to a mannose of an oligosaccharide linked to the Asn of the Fc fragment in preparation of a protein conjugate by using an α1,3 fucosyltransferase (e.g. an α1,3 fucosyltransferase derivated from Helicobacter pylori). For example, a protein comprising the -GlcNAc(Fuc)_{b}-GalX linked directly to the N297 of the Fc fragment may have much higher conversion efficiency compared to that comprising a -GlcNAc-GalX linked to a mannose of an oligosaccharide linked to the Asn of the Fc fragment in preparation of a protein conjugate by using a Helicobacter pylori α1,3 fucosyltransferases (i.e. an α1,3 fucosyltransferase derivated from Helicobacter pylori). For example, an Fc-fusion protein or an antibody with a Fc fragment comprising a -GlcNAc-Gal may have much higher conversion efficiency compared to that comprising a -GlcNAc-Gal linked to a mannose of an oligosaccharide linked to the N297 of the Fc fragment in preparation of a protein conjugate by using Helicobacter pylori α1,3 fucosyltransferases. For example, in Example 33, the trastuzumab-(Galβ1,4)GlcNAc showed strikingly higher conversion efficiency than trastuzumab-G₂F.

In the present disclosure, a protein comprising a -GlcNAc-GalX may have higher conversion efficiency compared to that comprising a -GlcNAc(Fuc)-GalX in the preparation of a protein conjugate by using an α1,3 fucosyltransferase (e.g., an α1,3 fucosyltransferase derivated from Helicobacter pylori), wherein the -GlcNAc-GalX or -GlcNAc(Fuc)-GalX is directly to the N297 of the Fc fragment, and the Fuc is linked to the GlcNAc through an α1,6 linkage. For example, an Fc-fusion protein or an antibody with a Fc fragment comprising a -GlcNAc-Gal may have higher conversion efficiency compared to that comprising a -GlcNAc(Fuc)-Gal in preparation of a protein conjugate by using an Helicobacter pylori α1,3 fucosyltransferase. For example, in Example 33, the trastuzumab-(Galβ1,4)GlcNAc showed higher conversion efficiency than trastuzumab-(Fucα1,6)(Galβ1,4)GlcNAc when using Helicobacter pylori α1,3 fucosyltransferases.

In another aspect, the present disclosure provides a method for preparing a protein conjugate, comprising contacting a protein conjugate of the present disclosure with one or more Y-(L′)ₑ-P_{F}′, wherein the Y comprises a functional moiety capable of reacting with the X_{F} and/or the X_{G}, L′ is a linker, e is 0 or 1, and the P_{F}′ is a biologically active molecule and/or a pharmaceutically active molecule.

The P_{F}′ may be the same or different as the P_{F} of the present disclosure. The L′ may be the same or different as any of the L₁ to Lₙ of the present disclosure. For example, in the present disclosure, the P_{F}′ may be a different molecule than the P_{F}, but they can be selected from the same group of molecules. Similarly, the L′ may be a different linker structure than any of the L₁ to Lₙ.

For example, The P_{F}′ may comprise a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof: In some cases, the P_{F}′ is a pharmaceutically active molecule. For example, the P_{F}′ may comprise a cytotoxin, an agonist, an antagonist, an antiviral agent, an antibacterial agent, an oligonucleotide, a polypeptide or any combination thereof: In some cases, the P_{F}′ comprises a cytotoxin or an agonist (such as a sting agonist, or a toll like receptor (such as TLR7/8) agonist). In some cases, the P_{F}′ comprises a DNA or RNA damaging agent, an RNA polymerase inhibitor, a topoisomerase inhibitor and/or a microtubule inhibitor. In some cases, the P_{F}′ comprises a pyrrolobenzodiazepine, an auristatin, a maytansinoids, a duocarmycin, a tubulysin, an enediyene, a doxorubicin, a pyrrole-based kinesin spindle protein inhibitor, a calicheamicin, an amanitin, a camptothecin and/or derivatives thereof. In some cases, the P_{F}′ comprises an MMAE, a DXd, T785 and/or their derivatives thereof:

For example, the L′ may be a linker of Formula (X): (FL′)_{x′}—(CL′)_{y}, (IV), the FL′ is a spacer moiety, the CL′ is a cleavable linker, x′ and y′ are independently 0 or 1. The right side of Formula (X) is linked to the Y, and the left side of Formula (X) is linked to the P_{F}′. For example, the FL′ side is linked to the Y, the CL′ side is linked to the P_{F}′.

The FL′ may be a spacer moiety selected from the group consisting of: C₁-C₁₀₀ alkylene, C₃-C₁₀₀ cycloalkylene, C₂-C₁₀₀ alkenylene, C₅-C₁₀₀ cycloalkenylene, C₂-C₁₀₀ alkynylene, C₆-C₁₀₀ cycloalkynylene, C₂-C₁₀₀ (hetero)arylene, C₃-C₁₀₀ (hetero)arylalkylene, C₃-C₁₀₀ alkyl(hetero)arylene, their derivatives and any combination thereof, wherein each of said alkylene, cycloalkylene, alkenylene, cycloalkenylene, alkynylene, cycloalkynylene, (hetero)arylene, (hetero)arylalkylene and alkyl(hetero)arylene is independently optionally substituted by one or more Rs¹ and/or is independently optionally interrupted by one or more Rs². For example, one or more of the alkylene, cycloalkylene, alkenylene, cycloalkenylene, alkynylene, cycloalkynylene, (hetero)arylene, (hetero)arylalkylene and alkyl(hetero)arylene may be substituted by one or more Rs¹. In some cases, one or more Rs² may be inserted in the alkylene, cycloalkylene, alkenylene, cycloalkenylene, alkynylene, cycloalkynylene, (hetero)arylene, (hetero)arylalkylene and/or alkyl(hetero)arylene. For example, an alkylene may be inserted by one or more —O— to become a -PEG-.

Each Rs¹ may independently be selected from the group consisting of halogen, —OH, —NH₂ and —COOH, and each Rs² may independently be selected from the group consisting of —O—, —S—,

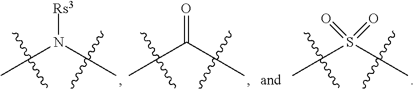

Rs³ may be selected from the group consisting of hydrogen, C₁-C₂₄ alkyl, C₂-C₂₄ alkenyl, C₂-C₂₄ alkynyl and C₃-C₂₄ cycloalkyl.

In some cases, the FL′ is a spacer moiety selected from the group consisting of:

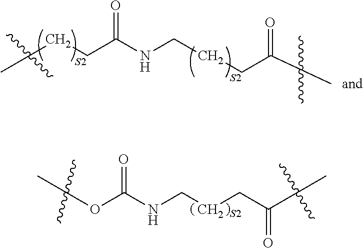

wherein said S2 may be independently 0-50. Each said —CH₂—(—CH₂— in the parentheses) may independently be replaced by a —O—, with the proviso that two or more consecutive —CH₂— are not simultaneously replaced by —O—. Accordingly, when one —CH₂— is replaced by a —O—, its immediate neighboring —CH₂— to the left and to the right may not be replaced by —O—. The left side of the structure of the FL′ may be linked to the Y, and the right side of the structure of the FL may be linked to the CL or the P_{F}′.

In some cases, the FL′ is a spacer moiety selected from the group consisting of:

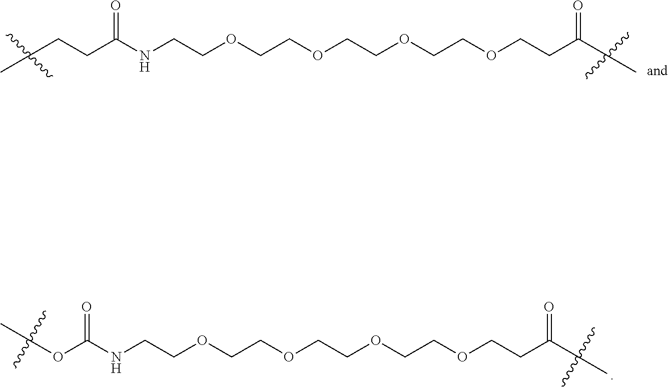 [SMILES: *CCC(=O)NCCOCCOCCOCCOCCC(*)=O ; *OC(=O)NCCOCCOCCOCCOCCC(*)=O]

The left side of the structure of the FL′ may be linked to the Y, and the right side of the structure of the FL may be linked to the CL or the P_{F}′.

The CL′ may be an acid-labile linker, a redox-active linker, a photo-active linker and/or a proteolytically cleavable linker. In some cases, the CL may be a vc-PAB-linker and/or a GGFG-linker.

In the present disclosure, Y may comprise a functional moiety capable of bioorthogonally reacting with the X_{F} and/or the X_{G} of the present disclosure. In some cases. Y is a functional moiety capable of bioorthogonally reacting with the X_{F} and/or the X_{G} of the present disclosure. In some cases, the Y only bioorthogonally reacts with the X_{F}. In some cases, the Y only bioorthogonally reacts with the X_{G}. In some cases, the Y bioorthogonally reacts with both the X_{F} and the X_{G}.

For example, Y may comprise a functional moiety selected from the group consisting of azido group, terminal alkynyl group, cyclic alkynyl group, tetrazinyl group, 1,2,4-trazinyl group, terminal alkenyl group, cyclic alkenyl group, ketone group, aldehyde group, hydroxyl amino group, sulfydryl group, N-maleimide group and their functional derivatives. The functional derivatives may retain similar or higher activities as the above functional moieties in a bioorthogonal ligation reaction.

In some cases, the Y may comprise a functional moiety selected from the group consisting of:

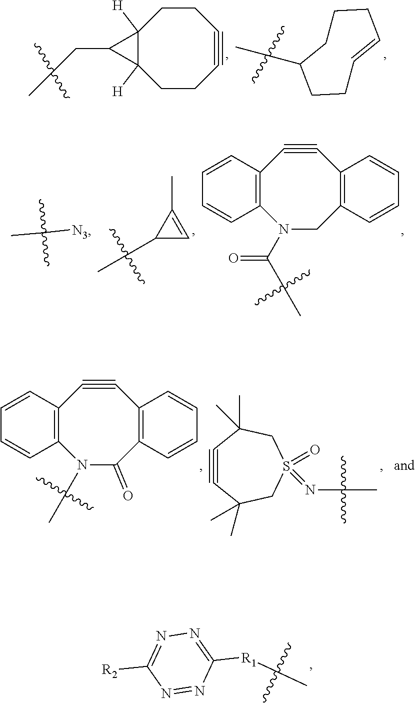

wherein R₁ and R₂ are as defined in present disclosure.

In some cases, the Y may comprise a functional moiety selected from the group consisting of

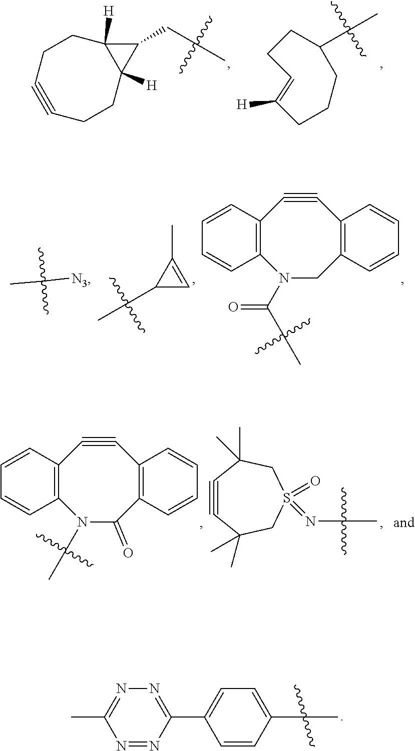

When the protein conjugate of the present disclosure reacts with multiple Y-(L′)ₑ-P_{F}′, the Y, L′, e, and P_{F}′ in different Y-(L′)ₑ-P_{F}′ may independently be identical or different. For example, some of the Y may be capable of only bioorthogonally reacting with X_{F}, some of the Y may be capable of only bioorthogonally reacting with the X_{G}, and some of the Y may be capable of bioorthogonally reacting with both the X_{F} and the X_{G}.

For example, when the X_{F} and/or the X_{G} comprise

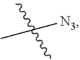 [SMILES: *N=[N+]=[N-]]

Y may comprise

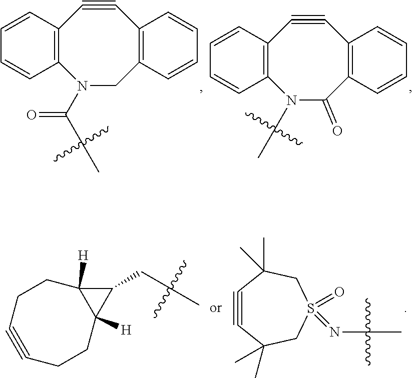

For example, when the X_{F} and/or the X_{G} comprise

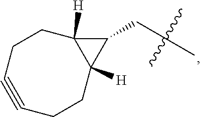 [SMILES: *C[C@H]1[C@H]2CCC#CCC[C@@H]12]

Y may comprise the

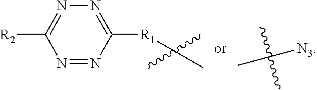

For example, when the X_{F} and/or the X_{G} comprise or

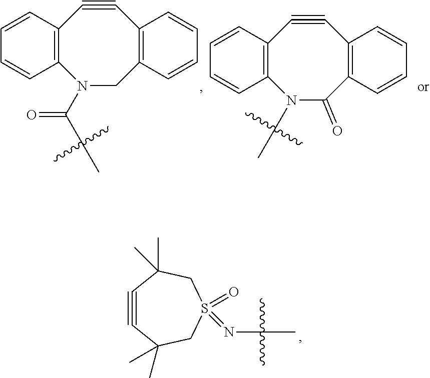

Y may comprise

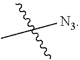 [SMILES: *N=[N+]=[N-]]

For example, when the X_{F} and/or the X_{G} comprise

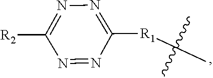

Y may comprise

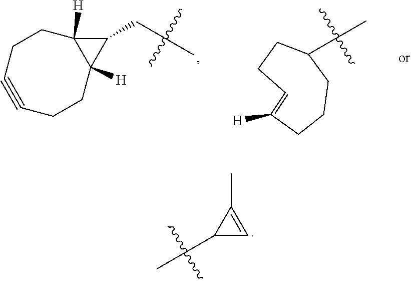

For example, when the X_{F} and/or the X_{G} comprise

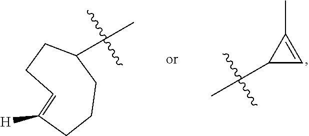

Y may comprise the

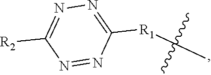

When there are multiple X_{F}, the above definitions regarding the X_{F} and the Y apply, if at least one X_{F} satisfies the above requirement.

Examples 34-42 provide some example for preparing protein conjugates by reacting the protein conjugate of present disclosure with Y-(L′)ₑ-P_{F}′.

The present disclosure also provides use of the Q-Fuc* of the present application in the preparation of a protein conjugate.

In the preparation method of the present disclosure, almost none-organic solvent would be necessary by using a Q-Fuc*, even when multiple highly hydrophobic active molecules are comprised in the Fuc*.

The present disclosure also provides a protein conjugate obtained with the method according to the present disclosure.

### Composition and Medical Use

In another aspect, the present disclosure provides a composition comprising the protein conjugate of the present disclosure.

In some embodiments, the protein conjugates comprised in the composition have at least 2 average MARs. The term “average MAR” (e.g., average MₙAR) generally refers to an average AM-to-antibody (such as AMₙ-to-antibody) ratio in a composition comprising two or more protein conjugates. The MAR and/or DAR may be measured by LC-MS or HIC-HPLC analysis.

In some embodiments, the protein conjugates comprised in the composition have at least 2 average MARs, and each of the average MARs is about 2. For example, each MAR (or DAR, when the AM comprises a pharmaceutically active molecule, such as a drug, e.g., a cytotoxin or an agonist) may be about 2 (e.g., 1.9-2, 1.8-2, 1.7-2, 1.6-2, 1.5-2, 1.2-2 or 1-2). For example, each average MAR may be 1.8-2. For example, each average MAR may be 1.6-2. For example, each average MAR may be 1.2-2.

In some embodiments, the protein conjugates comprised in the composition have at least 2 average MARs, and each of the average MAR is about 4. For example, each MAR (or DAR, when the AM comprises a pharmaceutically active molecule, such as a drug, e.g., a cytotoxin or an agonist) may be about 4 (e.g., 3.8-4, 3.6-4, 3.2-4, or 2.8-4). For example, each average MAR may be 3.6-4. For example, each average MAR may be 3.2-4. For example, each average MAR may be 2.8-4.

The composition may be a pharmaceutical composition.

In some cases, the composition may comprise a pharmaceutically acceptable carrier. The pharmaceutically acceptable carrier may be a carrier non-toxic to the cells or subjects exposed to them at an administrated dose and concentration. The composition may be used for preventing or treating a disease.

In another aspect, the present disclosure provides a method for preventing or treating a disease, comprising administrating to a subject in need thereof a protein conjugate or a composition of the present disclosure.

In another aspect, the present disclosure provides use of a protein conjugate or a composition of the present disclosure in the preparation of a medicament for preventing or treating a disease.

In the present disclosure, “preventing or treating a disease” may include killing or inhibiting the growth or replication of a tumor cell or cancer cell, treating cancer, treating a pre-cancerous condition, killing or inhibiting the growth or replication of a cell that expresses an auto-immune antibody, treating an autoimmune disease, treating an infectious disease, preventing the multiplication of a tumor cell or cancer cell, preventing cancer, preventing the multiplication of a cell that expresses an auto-immune antibody, preventing an autoimmune disease, and preventing an infectious disease.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., pl, picoliter(s); s or see, second(s); min, minute(s); h or hr, hour(s); aa, amino acid(s); nt, nucleotide(s); i.m., intramuscular(ly); i.p., intraperitoneal(ly); s.c., subcutaneous(ly); r.t., room temperature; and the like.

### Example 1 Synthesis of GDP-FAm

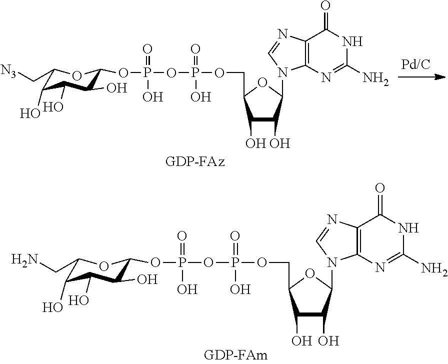

GDP-FAz was synthesized according to the reported procedure (Wu P., et al., Proc. Natl. Acad. Sci. USA 2009, 106, 16096), and purified through a Bio-Gel P-2 Gel column (Biorad). IRMS (ESI−) calculated for C₁₆H₂₄N₈O₁₅P₂(M−H⁺) 629.0764, found 629.0785.

To a clear solution of 100 mg (0.16 mmol) GDP-FAz in 8.75 mL MeOH/ddH₂O (1:1.5), 5 mg Pd/C (10%) was added. The air atmosphere was changed to H₂ by vacuum and refill, the H₂ pressure was kept at 0.28 MPa. The reaction was allowed for stirred 4 h and filtered through a 0.22 μM filter. Rotorvap and lyophilization give the product as a white solid (84.8 mg, yield 88%). IRMS (ESI−) calculated for C₁₆H₂₆N₆O₁₅P₂(M−H⁺) 603.0859, found 603.0874. ¹H NMR (400 MHz, D₂O) δ 8.10 (s, 1H), 5.92 (d, J=6.1 Hz, 1H), 4.97 (t, J=7.6, 1H), 4.76-4.73 (m, 1H), 4.51 (dd, J=5.2, 3.4 Hz, 1H), 4.37-4.34 (m, 1H), 4.23-4.21 (m, 2H), 3.96 (dd, J=9.6, 2.4 Hz, 1H), 3.92-3.91 (m, 1H), 3.70 (dd, J=10.0, 3.3 Hz, 1H), 3.63 (dd, J=10.0, 7.6 Hz, 1H), 3.31 (dd, J=13.4, 9.6 Hz, 1H), 3.24 (dd, J=13.4, 3.1 Hz, 1H).

### Example 2 Synthesis of GDP-FD1

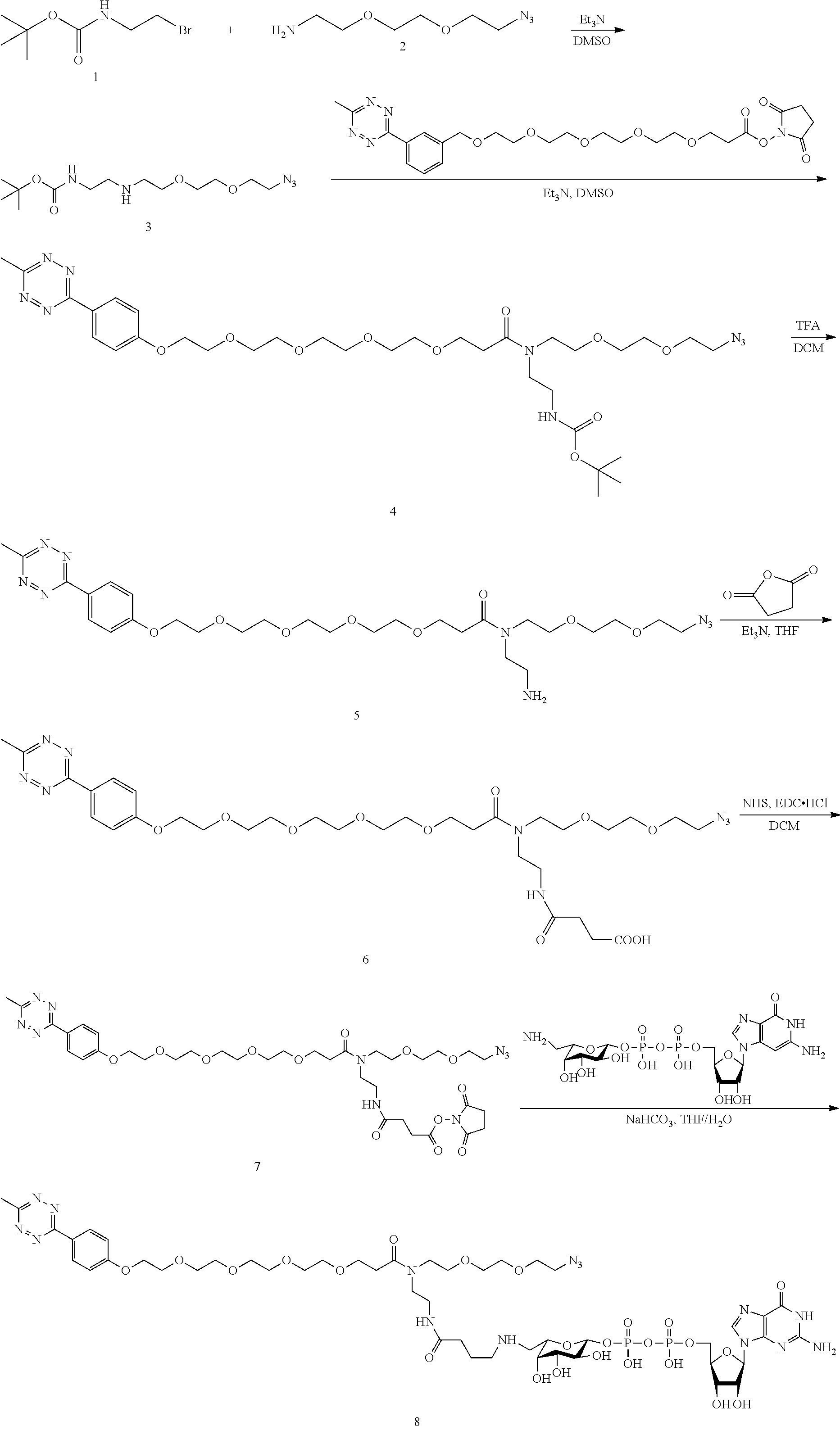

To a solution of 2 (2.2 g, 12.63 mmol) in 10 mL DMSO was added 1.75 ml Et₃N, and then 1 (1.41 g, 6.32 mmol) in 10 mL DMSO was added dropwise over 2 hours. The mixture was stirred at r.t. overnight. The product was further purified through a Prep-HPLC system to give 3 as a pale yellow oil (920.5 mg, 45.9%). HRMS (ESI+) calculated for C₁₃H₂₇N₅O₄ (M+H⁺) 318.2136, found 318.2128.

To a solution of 3 (469.5 mg, 1.48 mmol) in 7 mL DMSO was added 1.03 ml Et₃N, and then NHS-PEG₄-Tz (789.2 mg, 1.48 mmol) (Xi'an Dianhua Biotechnology Co., Ltd) in 3 mL DMSO was added. The mixture was stirred at r.t. for 5 h and monitored by TLC. The product was further purified through a Prep-HPLC system to give 4 as a pink solid (670 mg, yield 61.6%). HRMS (ESI+) calculated for C₃₃H₅₃N₉O₁₀ (M+Na⁺) 758.3808, found 758.3794.

To a solution of 4 (670 mg, 0.91 mmol) in 30 mL DCM was added 30 ml TFA. The mixture was stirred at r.t. for 1 h and monitored by TLC. The solvent was removed under reduced pressure to afford 5 as a crude product without further purification. HRMS (ESI+) calculated for C₂₈H₄₅N₉O₈ (M+H⁺) 636.3464, found 636.3462. To a solution of the crude product 5 in THF (30 mL) was added 126 μL TEA and succinic anhydride (273.2 mg, 2.73 mmol). The mixture was stirred at room temperature overnight and monitored by TLC. The product was further purified through a Prep-HPLC system to give 6 as a pink solid (567.3 mg, 84.8% in two steps). HRMS (ESI−) calculated for C₃₂H₄₉N₉O₁₁ (M−H⁺) 734.3479, found 734.3470.

To a solution of 6 (567 mg, 0.77 mmol) in 20 ml DCM was added NHS (265.9 mg, 2.31 mmol) and EDC·HCl (885.7 mg, 4.62 mmol). The mixture was stirred at r.t. for 3 h and monitored by TLC. The product was further purified through a Prep-HPLC system to give 7 as a pink solid (557.7 mg, 87.0%). HRMS (ESI+) calculated for C₃₆H₅₂N₁₀O₁₃ (M+Na⁺) 855.3608, found 855.3579.

To a solution of GDP-FAm (404.7 mg, 0.67 mmol) in 30 mL H₂O was added NaHCO₃ (112.6 mg, 1.34 mmol), and then 7 (557.7 mg, 0.67 mmol) in 30 mL THE was added. The mixture was stirred at r.t. for 5 h and monitored by TLC. The product was further purified through a Prep-HPLC system to generate the 8 (GDP-FD1) as a pink solid (371.1 mg, yield 41.9%). HRMS (ESI−) calculated for C₄₈H₇₃N₁₅O₂₅P₂(M−2H⁺)/2. 659.7116, found 659.7112. ¹H NMR (400 MHz, D₂O) δ 8.24-8.21 (m, 2H), 8.0 (s, 1H), 7.12-7.09 (m, 2H), 5.80 (dd, J=6.0, 3.2 Hz, 1H), 4.92 (t, J=7.8 Hz, 1H), 4.72-4.68 (m, 1H), 4.51-4.49 (m, 1H), 4.30-4.27 (m, 3H), 4.21-4.19 (m, 2H), 3.96-3.94 (m, 2H), 3.84 (d, J=3.3 Hz, 1H), 3.80-3.78 (m, 2H), 3.74-3.57 (m, 24H), 3.54-3.47 (m, 4H), 3.44-3.40 (m, 2H), 3.33-3.25 (m, 2H), 3.12-3.06 (m, 1H), 3.02 (s, 3H), 2.69-2.62 (m, 2H), 2.52-2.42 (m, 4H).

### Example 3 Synthesis of GDP-FD2

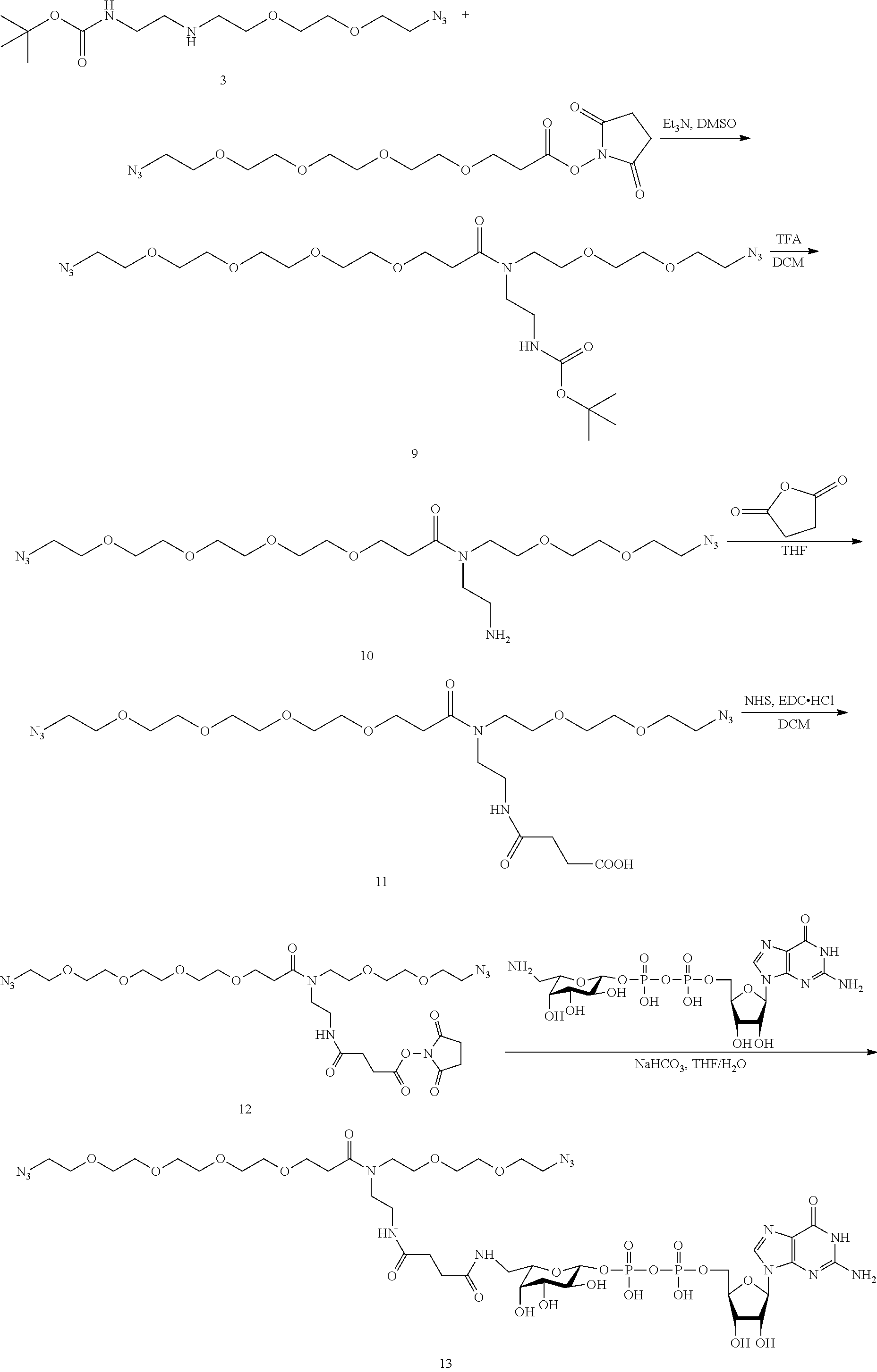

To a solution of 3 (190.3 mg, 0.6 mmol) in 3 mL DMSO was added 0.42 ml Et₃N, and then NHS-PEG₄-Az (232.9 mg, 0.6 mmol) (Xi'an Dianhua Biotechnology Co., Ltd) in 3 mL DMSO was added. The mixture was stirred at r.t. for 5 h and monitored by TLC. The product was further purified through a Prep-HPLC system to give 9 as a white solid (182 mg, yield 51.4%). HRMS (ESI−) calculated for C₂₄H₄₆N₈O₉ (M−H⁺) 589.3315, found 589.3312.

To a solution of 9 (182 mg, 0.31 mmol) in 10 mL DCM was added 10 ml TFA. The mixture was stirred at r.t. for 1 h and monitored by TLC. The solvent was removed under reduced pressure to afford 10 as a crude product without further purification. IRMS (ESI−) calculated for C₁₉H₃₈N₈O₇ (M−H⁺) 489.2791, found 489.2784. To a solution of the crude product 10 in THF (10 mL) was added 43 μL TEA and succinic anhydride (93.1 mg, 0.93 mmol). The mixture was stirred at room temperature overnight and monitored by TLC. The product was further purified through a Prep-HPLC system to give 11 as a white solid (81.1 mg, 44.3% in two steps). IRMS (ESI−) calculated for C₂₃H₄₂N₈O₁₀ (M−H⁺) 589.2951, found 589.2943.

To a solution of 11 (81.1 mg, 0.14 mmol) in 5 ml DCM was added NHS (48.3 mg, 0.42 mmol) and EDC·HCl (161.0 mg, 0.84 mmol). The mixture was stirred at r.t. for 3 h and monitored by TLC. The product was further purified through a Prep-HPLC system to give 12 as a white solid (85.0 mg, 88.3%). HRMS (ESI−) calculated for C₂₇H₄₅N₉O₁₂ (M+Cl⁻) 722.2882, found 722.2887.

To a solution of GDP-FAm (72.5 mg, 0.12 mmol) in 2.5 mL H₂O was added NaHCO₃ (20.2 mg, 0.24 mmol), and then 12 (85.0 mg, 0.12 mmol) in 2.5 mL THE was added. The mixture was stirred at r.t. for 5 h and monitored by TLC. The product was further purified through a Prep-HPLC system to generate the 13 (GDP-FD2) as a white solid (67.8 mg, yield 48.0%). HRMS (ESI−) calculated for C₃₉H₆₆N₁₄O₂₄P₂ (M−2H⁺)/2 587.1852, found 587.1865. ¹H NMR (400 MHz, D₂O) 8.09 (s, 1H), 5.91 (d, J=6.3 Hz, 1H), 4.91 (t, J=8.0 Hz, 1H), 4.77-4.75 (m, 1H), 4.52-4.50 (m, 1H), 4.34-4.32 (m, 1H), 4.21-4.19 (m, 2H), 3.84 (d, J=3.3 Hz, 1H), 3.79-3.75 (m, 2H), 3.70-3.65 (m, 24H), 3.27 (dd, J=14.0, 8.8 Hz, 1H), 3.17-3.14 (m, 4H), 3.12-3.06 (m, 4H), 2.90-2.86 (m, 4H), 2.76-2.69 (m, 2H), 2.53-2.47 (m, 4H).

### Example 4 Synthesis of GDP-FD3

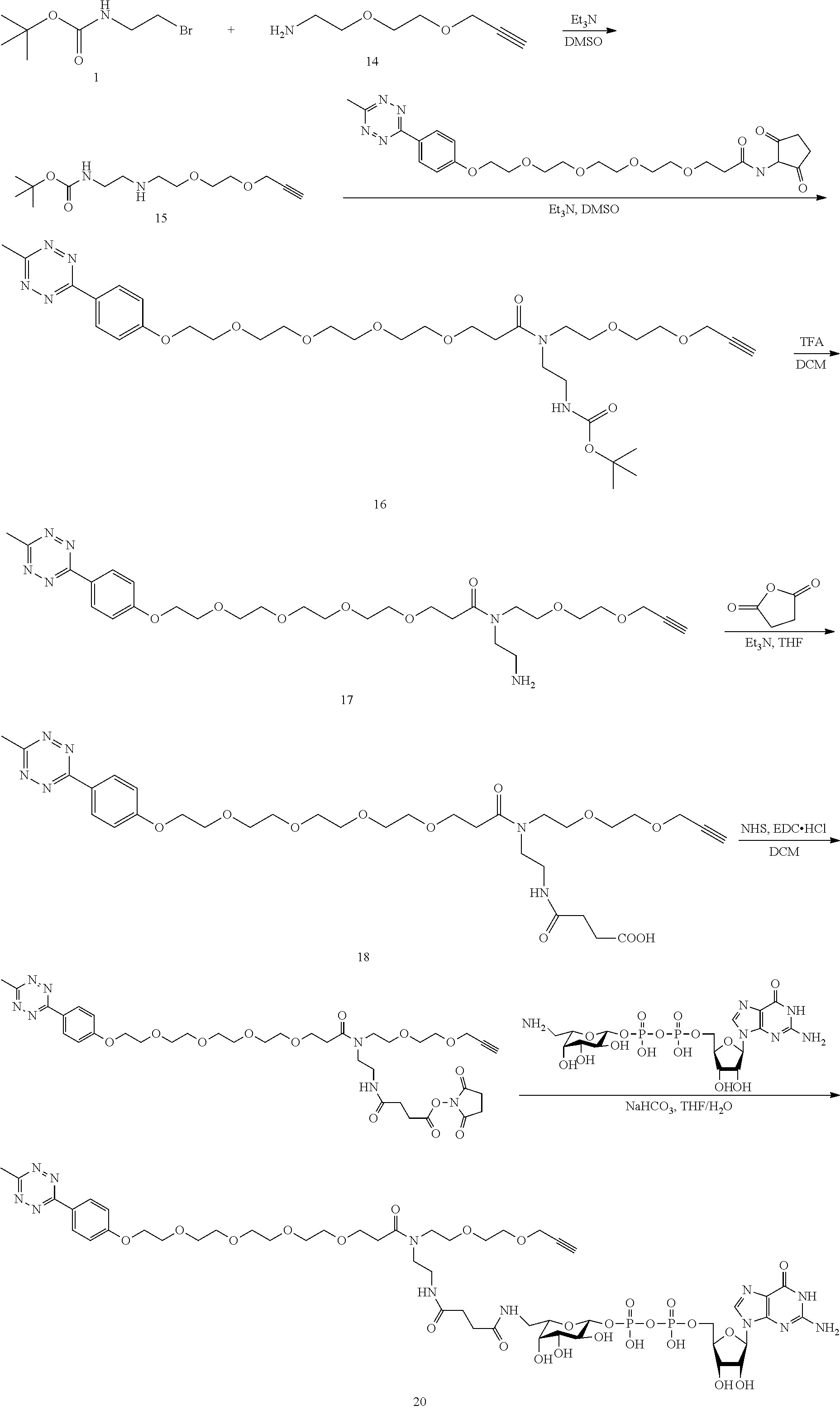

To a solution of 14 (286.2 mg, 2.0 mmol) in 5 mL DMSO was added 278 ul Et₃N, and then 1 (223.0 mg, 1.0 mmol) in 5 mL DMSO was added dropwise over 2 hours. The mixture was stirred at r.t. overnight. The product was further purified through a Prep-HPLC system to give 15 as a pale yellow oil (166.6 mg, 58.2%). HRMS (ESI+) calculated for C₁₄H₂₆N₂O₄ (M+H⁺) 287.1965, found 287.1948.

To a solution of 15 (166.6 mg, 0.58 mmol) in 5 mL DMSO was added 403.1 ul Et₃N, and then NHS-PEG₄-Tz (309.3 mg, 0.58 mmol) (Xi'an Dianhua Biotechnology Co., Ltd) in 3 mL DMSO was added. The mixture was stirred at r.t. for 5 h and monitored by TLC. The product was further purified through a Prep-HPLC system to give 16 as a pink solid (230.4 mg, yield 56.4%). HRMS (ESI+) calculated for C₃₄H₅₂N₆O₁₀ (M+Na⁺) 727.3637, found 727.3620.

To a solution of 16 (230.4 mg, 0.33 mmol) in 10 mL DCM was added 10 ml TFA. The mixture was stirred at r.t. for 1 h and monitored by TLC. The solvent was removed under reduced pressure to afford 17 as a crude product without further purification. HRMS (ESI+) calculated for C₂₉H₄₄N₆O₈ (M+H⁺) 605.3293, found 605.3281. To a solution of the crude product 17 in THE (10 mL) was added 45.9 μL TEA and succinic anhydride (99.1 mg, 0.99 mmol). The mixture was stirred at room temperature overnight and monitored by TLC. The product was further purified through a Prep-HPLC system to give 18 as a pink solid (182.5 mg, 78.5% in two steps). HRMS (ESI+) calculated for C₃₃H₄₈N₆O₁₁ (M+Na⁺) 727.3273, found 727.3267.

To a solution of 18 (182.5 mg, 0.26 mmol) in 10 ml DCM was added NHS (89.8 mg, 0.78 mmol) and EDC·HCl (299.1 mg, 1.56 mmol). The mixture was stirred at r.t. for 3 h and monitored by TLC. The product was further purified through a Prep-HPLC system to give 19 as a pink solid (193.4 mg, 92.8%). HRMS (ESI+) calculated for C₃₇H₅₁N₇O₁₃ (M+Na⁺) 824.3437, found 824.3420.

To a solution of GDP-FAm (145.0 mg, 0.24 mmol) in 10 mL H₂O was added NaHCO₃ (40.3 mg, 0.48 mmol), and then 19 (193.4 mg, 0.24 mmol) in 10 mL THE was added. The mixture was stirred at r.t. for 5 h and monitored by TLC. The product was further purified through a Prep-HPLC system to generate the 20 (GDP-FD3) as a pink solid (117.4 mg, yield 37.9%). HRMS (ESI−) calculated for C₄₉H₇₂N₁₂O₂₅P₂(M−2H⁺)/2 644.2031, found 644.2059. ¹H NMR (400 MHz, D₂O) 8.19-8.16 (m, 2H), 8.03 (s, 1H), 7.08-7.04 (m, 2H), 5.80 (dd, J=5.8, 3.2 Hz, 1H), 4.93 (t, J=7.8 Hz, 1H), 4.71-4.68 (m, 1H), 4.51-4.49 (m, 1H), 4.31-4.30 (m, 1H), 4.28-4.26 (m, 2H), 4.22-4.20 (m, 2H), 4.17 (dd, J=7.4, 2.4 Hz, 2H), 3.96-3.93 (m, 2H), 3.86 (d, J=3.3 Hz, 1H), 3.80-3.78 (m, 2H), 3.74-3.58 (m, 22H), 3.55-3.46 (m, 4H), 3.43-3.40 (m, 1H), 3.34-3.31 (m, 1H), 3.30-3.24 (m, 2H), 3.01 (s, 3H), 2.69-2.63 (m, 2H), 2.53-2.42 (m, 4H).

### Example 5 Synthesis of GDP-FD4

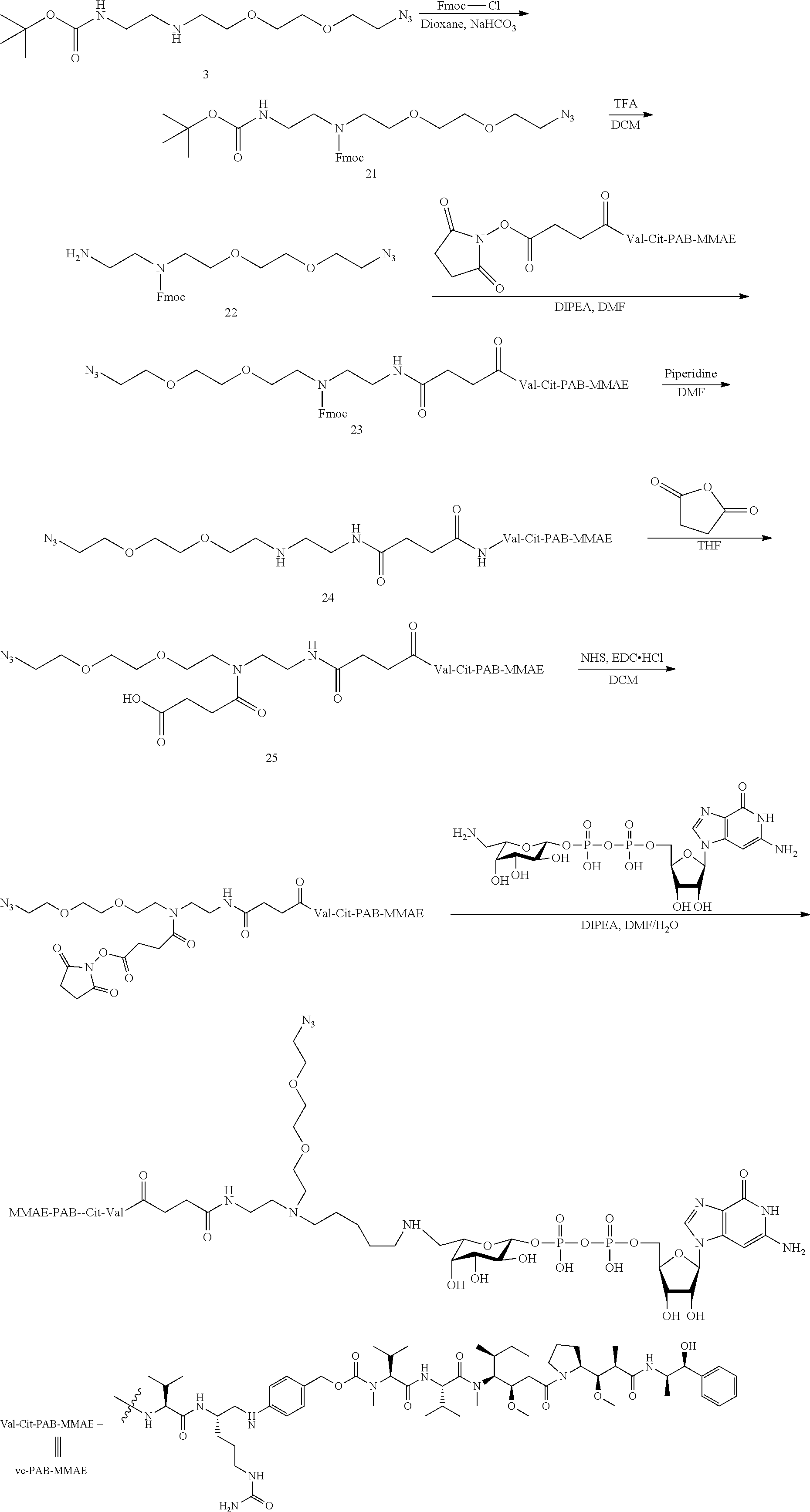

To a solution of 3 (317.2 mg, 1.0 mmol) in 5 mL dioxane was added 6 ml saturated NaHCO₃ (aq), and then Fmoc-Cl (310.4, 1.2 mmol) was added. The mixture was stirred at r.t. for 4 h and monitored by TLC. The crude product was further purified through a column chromatography to generate the 21 as a pale yellow oil (390.4 mg, yield 72.4%).

To a solution of 21 (390.4 mg, 0.72 mmol) in 10 mL DCM was added 10 ml TFA. The mixture was stirred at r.t. for 1 h and monitored by TLC. The solvent was removed under reduced pressure to afford 22 as a crude product without further purification. IRMS (ESI+) calculated for C₂₃H₂₉NsO₄ (M+H⁺) 440.2292, found 440.2278. To a solution of the crude product 22 in DMF (8 mL) was added 125.4 μL DIPEA and OSu-Suc-vc-PAB-MMAE (950.2 mg, 0.72 mmol). The mixture was stirred at room temperature overnight and monitored by TLC. The product was further purified through a Prep-HPLC system to give 23 as a white solid (442.7 mg, 37.4% in two steps). IRMS (ESI+) calculated for C₈₅H₁₂₅N₁₅O₁₈ (M+Na⁺) 1666.9219, found 1666.9180.

To a solution of 23 (442.7 mg, 0.27 mmol) in 8 mL DMF was added 2 ml piperidine. The mixture was stirred at r.t. for 1 h and monitored by TLC. The solvent was removed under reduced pressure to afford 24 as a white solid (300.6 mg, 78.3%). HRMS (ESI+) calculated for C₇₀H₁₁₅N₁₅O₁₆ (M+Na⁺) 1444.8538, found 1444.8506.

To a solution of 24 (300.6 mg, 0.21 mmol) in THE (20 mL) was added 29.2 μL TEA and succinic anhydride (63.0 mg, 0.63 mmol). The mixture was stirred at room temperature overnight and monitored by TLC. The product was further purified through a Prep-HPLC system to give 25 as a white solid (296.9 mg, 92.9%). HRMS (ESI+) calculated for C₇₄H₁₁₉N₁₅O₁₉ (M+Na⁺) 1544.8699, found 1544.8684.

To a solution of 25 (296.9 mg, 0.20 mmol) in 20 ml DCM was added NHS (69.1 mg, 0.6 mmol) and EDC·HCl (230.0 mg, 1.2 mmol). The mixture was stirred at r.t. for 3 h and monitored by TLC. The product was further purified through a Prep-HPLC system to give 26 as a white solid (287.2 mg, 88.7%). HRMS (ESI+) calculated for C₇₈H₁₂₂N₁₆O₂₁ (M+Na⁺) 1641.8863, found 1641.8845.

To a solution of GDP-FAm (108.7 mg, 0.18 mmol) in 9 mL H₂O was added NaHCO₃ (30.2 mg, 0.36 mmol), and then 26 (287.2 mg, 0.18 mmol) in 9 mL DMF was added. The mixture was stirred at r.t. for 5 h and monitored by TLC. The product was further purified through a Prep-HPLC system to generate the 27 (GDP-FD4) as a white solid (101.7 mg, yield 26.8%). HRMS (ESI−) calculated for C₉₀H₁₄₃N₂₁O₃₃P₂ (M−2H⁺)/2 1052.9744, found 1052.9777.

### Example 6 Synthesis of OSu-Suc-vc-PAB-MMAE

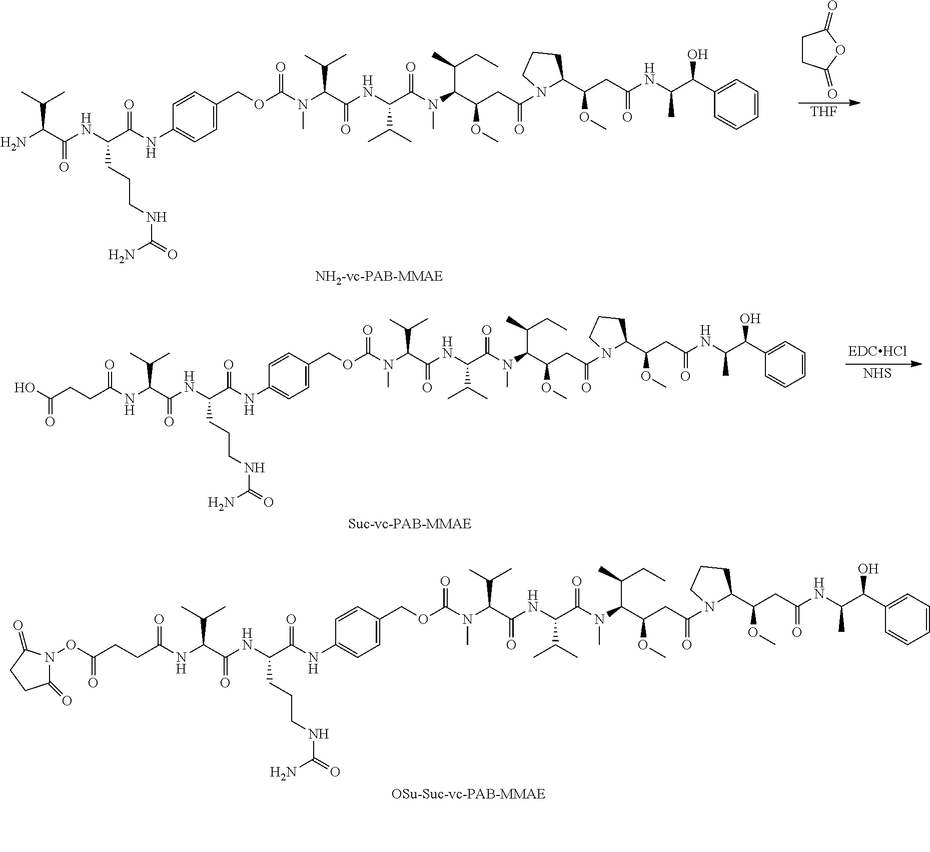

NH₂-vc-PAB-MMAE was synthesized according to the reported procedure (Tang, F., et al. Org. Biomol. Chem. 2016, 14, 9501).

To a solution of NH₂-vc-PAB-MMAE (1.68 g, 1.5 mmol) in DMF (30 mL) and THE (30 mL) was added succinic anhydride (450.3 mg, 4.5 mmol). The mixture was stirred at r.t. for 3 h and monitored by TLC. The product was further purified through a Prep-HPLC system to generate the Suc-vc-PAB-MMAE as a white solid (1.38 g, yield 75.3%). HRMS (ESI−) calculated for C₆₂H₉₈N₁₀O₁₅ (M−H⁺) 1221.7140, found 1221.7146.

To a solution of Suc-vc-PAB-MMAE (1.38 g, 1.13 mmol) in DCM (20 mL) and THF (20 mL) was added NHS (390.2 mg, 3.39 mmol) and EDC·HCl (1.30 g, 6.78 mmol). The mixture was stirred at r.t. for 3 h and monitored by TLC. The product was further purified through a Prep-HPLC system to generate the OSu-Suc-vc-PAB-MMAE as a white solid (1.49 g, yield 76.6%). IRMS (ESI+) calculated for C₆₆H₁₀₁N₁₁O₁₇ (M+Na⁺) 1342.7269, found 1342.7283.

### Example 7 Synthesis of Propargyl-PEG₄-GGFG-DXd

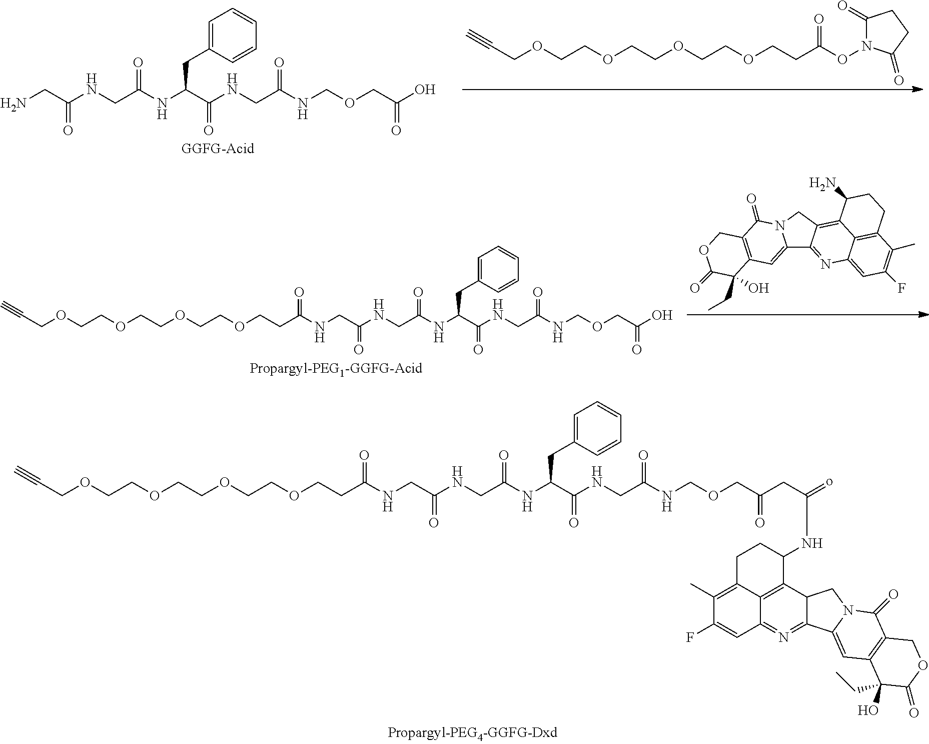

GGFG-Acid was synthesized according to the reported procedure (Yamaguchi, T., et al., EP3677589A1).

To a solution of GGFG-Acid (98.4 mg, 0.23 mmol) in DMF (5 ml) was added DIPEA (0.2 ml) and propargyl-PEG₄-OSu (99.6 mg, 0.28 mmol). The mixture was stirred at r.t. for overnight and monitored by TLC. The crude product was further purified through a Prep-HPLC system to generate the propargyl-PEG₄-GGFG Acid as a white solid (114.8 mg, 75.0%). IRMS (ESI−) calculated for C₃₀H₄₃N₅O₁₂ (M−H⁺) 664.2835, found 664.2808.

To a solution of propargyl-PEG₄-GGFG Acid (66.6 mg, 0.1 mmol) in DMF (5 ml) was added DIPEA (0.1 ml), Exatecan (43.5 mg, 0.1 mmol) and PyBOP (104.9 mg, 0.2 mmol). The mixture was stirred at r.t. for 2 h and monitored by TLC. The crude product was further purified through a Prep-HPLC system to generate the propargyl-PEG₄-GGFG-DXd as a light yellow solid (70.6 mg, 65.2%). HRMS (ESI+) calculated for C₅₄H₆₃FN₈O₁₅ (M+Na⁺) 1105.4289, found 1105.4255.

### Example 8 Synthesis of GDP-FD5

To a solution of 100 μL 27 (25 mM) in ddH₂O/DMSO (375 μL/440 μL), was added 2.5 μL CuSO₄ (100 mM), 10 μL BTTP (50 mM), 60 μL propargyl-PEG₄-GGFG-DXd (50 mM in DMSO) and 12.5 μL ascorbate sodium (100 mM in ddH₂O). The reaction as allowed for stirring at r.t. for 5 h and monitored by TLC. Then, 2 mM BCS was added to quench the reaction and the solvent was removed under reduced pressure. The crude product was further purified through a Prep-HPLC system to generate the product as a light yellow solid (5.4 mg, 67.7%). HRMS (ESI−) calculated for C₁₄₄H₂₀₆FN₂₉O₄₈P₂ (M−2H⁺)/2 1594.1942, Found 1594.1941.

### Example 9 Synthesis of GDP-FD6

To a solution of 100 μL 27 (25 mM) in ddH₂O/DMSO (375 μL/440 μL), was added 2.5 μL CuSO₄ (100 mM), 10 μL BTTP (50 mM), 60 μL propargyl-PEG₄-vc-PAB-MMAE (Levena Biopharma) (50 mM in DMSO) and 12.5 μL ascorbate sodium (100 mM in ddH₂O). The reaction as allowed for stirring at r.t. for 5 h and monitored by TLC. Then, 2 mM BCS was added to quench the reaction and the solvent was removed under reduced pressure. The crude product was further purified through a Prep-HPLC system to generate the product as a white solid (6.9 mg, 79.4%). HRMS (ESI−) calculated for C₁₆₀H₂₅₅N₃₁O₅₀P₂ (M−2H⁺)/2 1735.3847, Found 1735.3829.

### Example 10 Synthesis of GDP-FD7

To a solution of 300 μL 13 (25 mM) in ddH₂O/DMSO (1050 μL/1140 μL), was added 15 μL CuSO₄ (100 mM), 60 μL BTTP (50 mM), 360 μL propargyl-PEG₄-vc-PAB-MMAE (Levena Biopharma) (50 mM in DMSO) and 75 μL ascorbate sodium (100 mM in ddH₂O). The reaction was allowed for stirring at r.t. for 5 h and monitored by TLC. Then, 2 mM BCS was added to quench the reaction and the solvent was removed under reduced pressure. The crude product was further purified through a Prep-HPLC system to generate the product as a white solid (22.8 mg, 78.4%). HRMS (ESI—) calculated for C₁₇₉H₂₉₀N₃₄O₅₈P₂ (M−2H⁺)/2 1953.0093, found 1953.0063.

### Example 11 Synthesis of GDP-FD8

To a solution of 300 μL 13 (25 mM) in ddH₂O/DMSO (1050 μL/1140 μL), was added 15 μL CuSO₄ (100 mM), 60 μL BTTP (50 mM), 360 μL propargyl-PEG₄-GGFG-DXd (50 mM in DMSO) and 75 μL ascorbate sodium (100 mM in ddH₂O). The reaction was allowed for stirring at r.t. for 5 h and monitored by TLC. Then, 2 mM BCS was added to quench the reaction and the solvent was removed under reduced pressure. The crude product was further purified through a Prep-HPLC system to generate the product as a light yellow solid (16.8 mg, 67.6%). HRMS (ESI−) calculated for C₁₄₇H₁₉₂F_{2N30}O₅₄P₂ (M−2H⁺)/2 1670.6283, Found 1670.6271.

### Example 12 Synthesis of Propargyl-PEG₄-T785

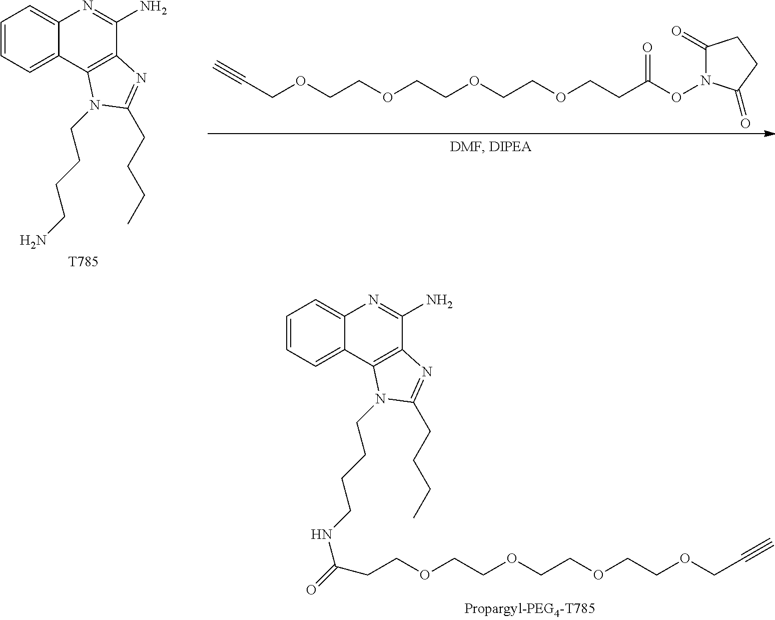

T785 was synthesized according to the reported procedure (Brian, S., Research Square, DOI: 10.21203/rs.3.pex-1149/v1).

To a solution of T785 (100 mg, 0.32 mmol) in DMF (2 mL) was added Prop-PEG₄-NHS (138 mg, 0.38 mmol) and DIPEA (266 μL, 1.60 mmol). The mixture was stirred at r.t. overnight and monitored by TLC. The product was further purified by Prep-HPLC system to generate the product as a white solid (45 mg, yield 25.3%). LC-MS (ESI+) calculated for C₃₀H₄₃N₅O₅ (M+H⁺) 554.3337, found 554.6.

### Example 13 Synthesis of GDP-FD9

To a solution of 100 μL 27 (25 mM) in ddH₂O/DMSO (375 μL/440 μL), was added 2.5 μL CuSO₄ (100 mM), 10 μL BTTP (50 mM), 60 μL propargyl-PEG₄-T785 (50 mM in DMSO) and 12.5 μL ascorbate sodium (100 mM in ddH₂O). The reaction as allowed for stirring at r.t. for 5 h and monitored by TLC. Then, 2 mM BCS was added to quench the reaction and the solvent was removed under reduced pressure. The crude product was further purified through a Prep-HPLC system to generate the product as a white solid (5.4 mg, 81.2%). HRMS (ESI−) calculated for C₁₂₀H₁₈₆N₂₆O₃₈P₂ (M−2H⁺)/2 1330.1392, Found 1330.1367.

### Example 14 Synthesis of GDP-FD10

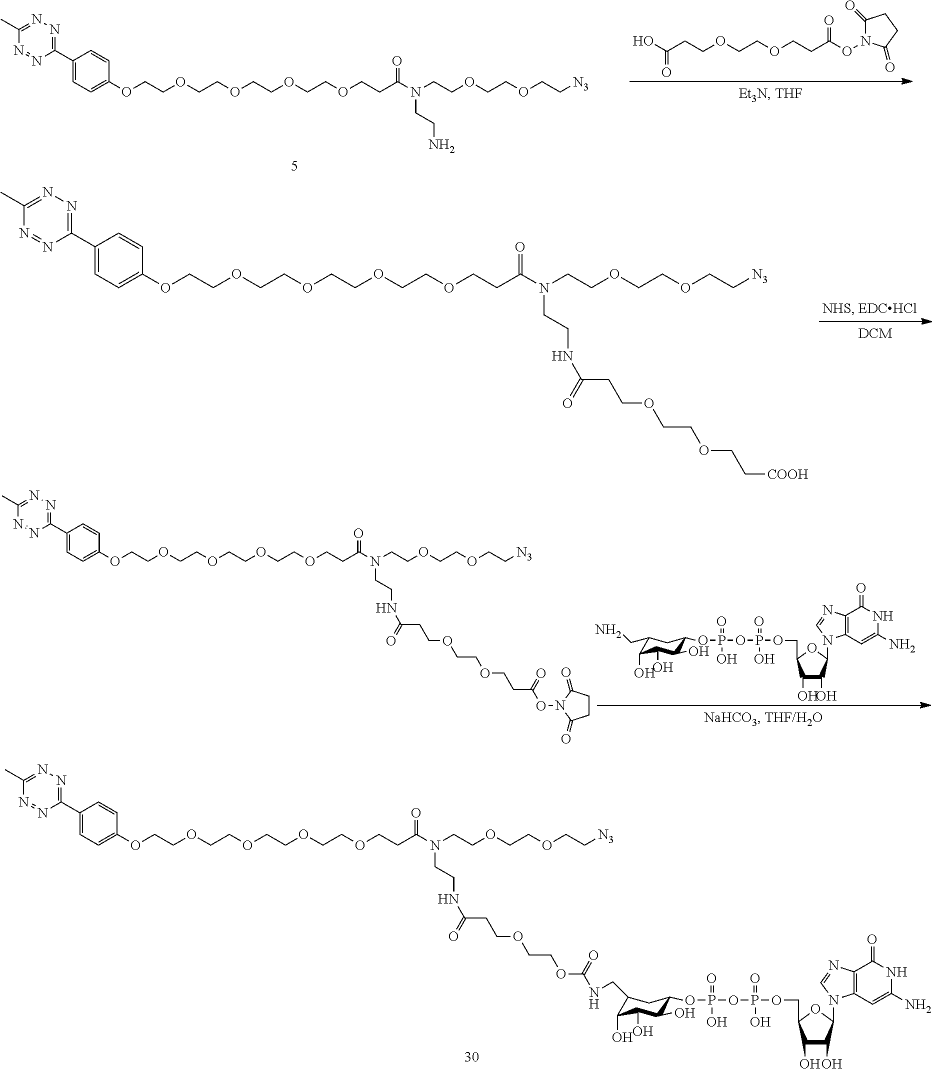

To a solution of the crude product 5 (31.8 mg, 0.05 mmol) in THE (3 mL) was added 13 μL TEA and NHS-PEG₂-COOH (30 mg, 0.1 mmol) (Xi'an Dianhua Biotechnology Co., Ltd). The mixture was stirred at room temperature overnight and monitored by TLC. The product was further purified through a Prep-HPLC system to give 28 as a pink solid (18.9 mg, 45.9%). HRMS (ESI−) calculated for C₃₆H₅₇N₉O₁₃ (M−H⁺) 822.4003, found 822.4028.

To a solution of 28 (9.5 mg, 0.0115 mmol) in 2 ml DCM was added NHS (4.0 mg, 0.0346 mmol) and EDC·HCl (13.3 mg, 0.0692 mmol). The mixture was stirred at r.t. for 3 h and monitored by TLC. The product was further purified through a Prep-HPLC system to give 29 as a pink solid (9.0 mg, 85.0%). HRMS (ESI+) calculated for C₄₀H₆₀N₁₀O₁₅ (M+Na⁺) 943.4132, found 943.4109.

To a solution of GDP-FAm (6.04 mg, 0.01 mmol) in 800 μL H₂O was added NaHCO₃ (1.7 mg, 0.02 mmol), and then 29 (9.0 mg, 0.01 mmol) in 800 μL H₂O THE was added. The mixture was stirred at r.t. for 5 h and monitored by TLC. The product was further purified through a Prep-HPLC system to generate the 30 (GDP-FD10) as a pink solid (3.0 mg, yield 21.3%). HRMS (ESI−) calculated for C₅₂H₈₁N₁₅O₂₇P₂(M−H⁺) 1408.4829, found 1408.4835.

### Example 15 Synthesis of TCO-PEG₄-vc-PAB-MMAE

To a solution of NH₂-vc-PAB-MMAE (30.0 mg, 0.027 mmol) in DMF (1.5 mL) were added DIPEA (100 L) and NHS-PEG₄-TCO (Xi'an Dianhua Biotechnology Co., Ltd) (16.5 mg, 0.032 mmol). The mixture was stirred at r.t. for overnight and monitored by TLC. The product was further purified through a Prep-HPLC system to generate the TCO-PEG₄-vc-PAB-MMAE as a white powder (21.7 mg, yield 53%). HRMS (ESI−) calculated for C₇₈H₁₂₇N₁₁O₁₉ (M−H⁺) 1520.9237, found 1520.9277.

### Example 16 Synthesis of DBCO-PEG4-Vc-PAB-Seco-DUBA (16-12)

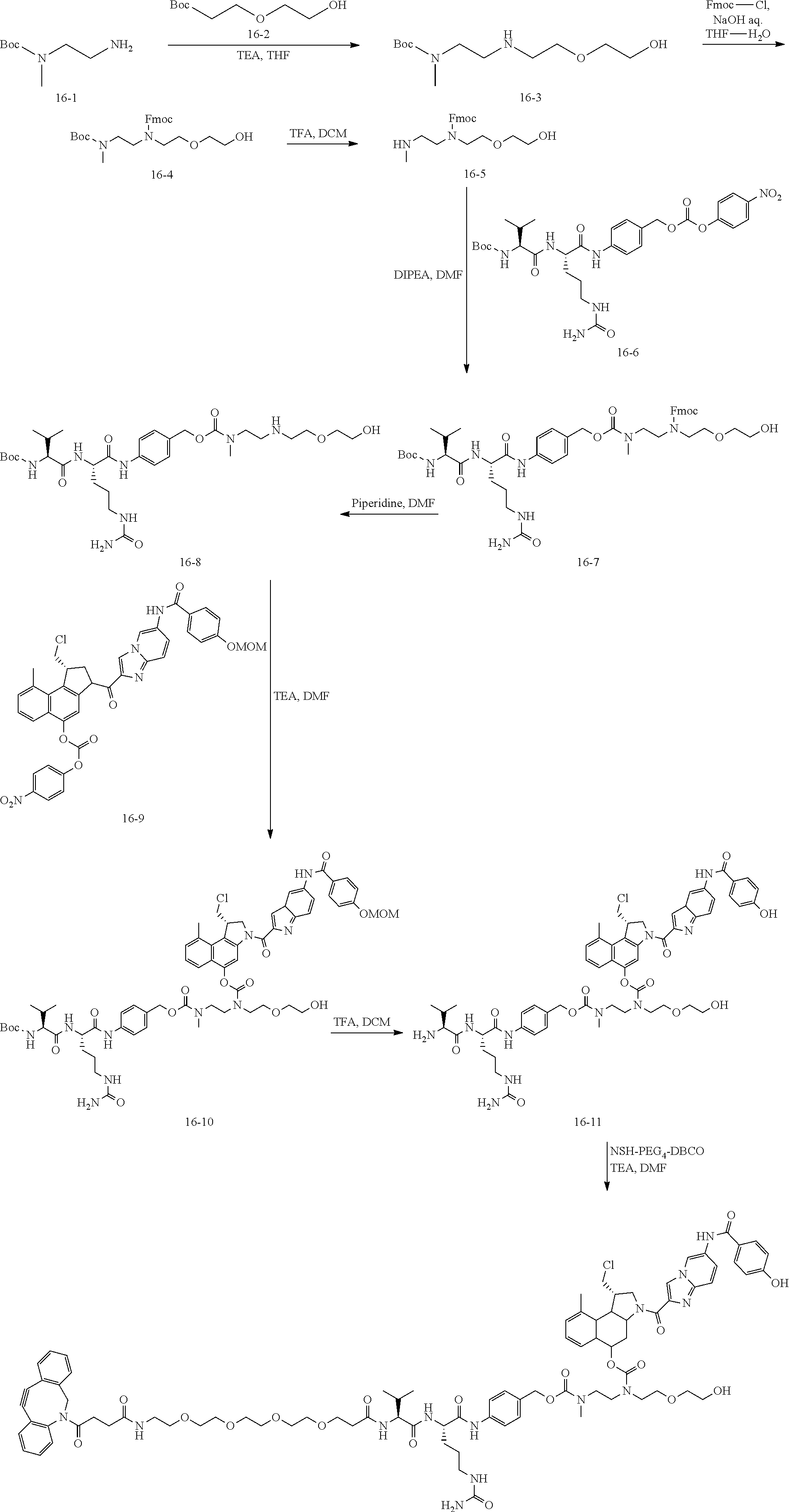

DBCO-PEG₄-vc-PAB-seco-DUBA was synthesized according to the route listed above.

Tert-butyl(2-((2-(2-hydroxyethoxy)ethyl)amino)ethyl)(methyl)carbamate (16-3). To a solution of tert-butyl(2-aminoethyl)(methyl)carbamate (16-1) (5.2 g, 30 mmol) in THE (60 mL) were added 5 g TEA. Then 2-(2-bromoethoxy)ethanol (16-2) (1.7 g, 10 mmol) was dropped to the mixture stepwise. The mixture was stirred at r.t. for 5 h and monitored by TLC. The solvent was removed under reduced pressure to generate the crude product 16-3.

Tert-butyl(2-((((9H-fluoren-9-yl)methoxy)carbonyl)(2-(2-hydroxyethoxy)ethyl)amino)ethyl)(methyl)carbamate (16-4). To a solution of all of the crude product 16-3 in THE (30 mL) and NaOH aq. (80 mL, 1N) were added Fmoc-Cl (7.8 g, 30.2 mmol) at r.t. The mixture was stirred at r.t. for 1 h and monitored by TLC. The solvent was then removed under reduced pressure. The residue was dissolved in 200 ml ethyl acetate and wash with washed with saturated NaHCO₃ solution and water respectively, followed by dried with Na₂SO₄. The crude product was further purified through a column chromatography to yield the 16-4 (3.5 g, yield 72% in two steps) as a light-yellow liquid.

(9H-fluoren-9-yl)methyl(2-(2-hydroxyethoxy)ethyl)(2-(methylamino)ethyl)carbamate (16-5). To a solution of 16-4 (3.4 g, 7.0 mmol) in DCM (15 mL) were added 20 ml TFA. The mixture was stirred at r.t. for 4 h and monitored by TLC. The solvent was removed under reduced pressure. The crude product was further purified through a column chromatography to yield 16-5 (0.88 g, yield 33%) as a colorless liquid. HRMS (ESI+) calculated for C₂₂H₂₈N₂O₄ (M+H⁺) 385.2122, found 385.2109.

4-((S)-2-((S)-2-((tert-butoxycarbonyl)amino)-3-methylbutanamido)-5-ureidopentanamido)benzyl (2-((2-(2-hydroxyethoxy)ethyl)amino)ethyl)(methyl) carbamate (16-8). To a solution of 16-6 Boc-vc-PAB-PNP (Tsbiochem) (1.5 g, 2.3 mmol) in DMF (5 mL) were added DIPEA (1.3 mL) and 16-5 (880 mg, 2.3 mmol). The mixture was stirred at r.t. for overnight and monitored by TLC. Then 3 ml piperidine was added to the mixture and stirred for another 4 h. The product was further purified through a Prep-HPLC system to generate the 16-8 as a white solid (736 mg. yield 48%). IRMS (ESI−) calculated for C₃₁H₅₃N₇O₉ (M−H⁺) 666.3832, found 666.3837.

4-((S)-2-((S)-2-((tert-butoxycarbonyl)amino)-3-methylbutanamido)-5-ureidopentanamido)benzyl(2-(((((S)-1-(chloromethyl)-3-(6-(4-(methoxymethoxy)benzamido)imidazo[1,2-a]pyridine-2-carbonyl)-9-methyl-2,3-dihydro-1H-benzo[e]indol-5-yl)oxy)carbonyl)(2-(2-hydroxyethoxy)ethyl)amino)ethyl)(methyl)carbamate (16-10). The PNP-seco-DUBA (16-9) was synthesized according to the reported procedure (Beusker P. H., et al., Mol. Pharmaceutics 2015, 12, 1813). To a solution of 16-9 (125 mg, 0.17 mmol) in DMF (5 mL) were added 130 μL TEA and 136 mg 16-8 (0.20 mmol). The mixture was stirred at r.t. for overnight and monitored by TLC. The product was further purified through a Prep-HPLC system to generate the 16-10 as a white solid (71 mg. yield 33%). HRMS (ESI−) calculated for C₆₃H₇₈C₁N₁₁O₁₅ (M−H⁺) 1262.5295, found 1262.5287.

4-((S)-2-((S)-2-amino-3-methylbutanamido)-5-ureidopentanamido)benzyl(2-(((((S)-1-(chloromethyl)-3-(6-(4-hydroxybenzamido)imidazo[1,2-a]pyridine-2-carbonyl)-9-methyl-2,3-dihydro-1H-benzo[e]indol-5-yl)oxy)carbonyl)(2-(2-hydroxyethoxy)ethyl)amino)ethyl)(methyl)carbamate (16-11). To a solution of 16-10 (71 mg, 0.056 mmol) in DCM (2 mL) were added 3 ml TFA. The mixture was stirred at r.t. for 3 h and monitored by TLC. The solvent was removed under reduced pressure to afford the 16-11 as a crude product (58 mg) without further purification.

DBCO-PEG₄-vc-PAB-seco-DUBA (16-12). To a solution of the crude product 16-11 (25 mg) in DMF (1.5 mL) were added 100 μL TEA and 20 mg NHS-PEG₄-DBCO ester (Xi'an Dianhua Biotechnology Co., Ltd) (0.03 mmol). The mixture was stirred at room temperature overnight and monitored by TLC. The product was further purified through a Prep-HPLC system. Concentration and lyophilization to give 16-12 as a white powder (15.2 mg). HRMS (ESI−) calculated for C₈₆H₁₀₀C₁N₁₃O₁₉ (M−H⁺) 1653.6908, found 1653.6948.

### Example 17 Synthesis of GDP-FAmP₄Biotin

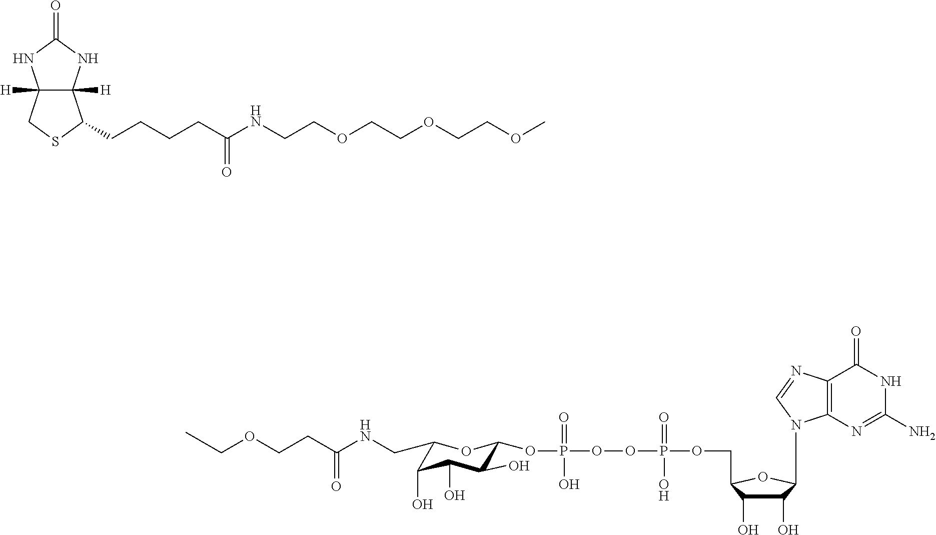

To a solution of 500 μL GDP-FAm (100 mM in ddH₂O) in 1.5 mL ddH₂O were added 500 μL NaHCO₃ (200 mM), 1.95 mL THE and 550 μL NHS-PEG₄-Biotin (Click Chemistry Tools) (100 mM in THF) were added. The reaction was stirred at r.t. for 4 h and monitored by TLC. The solvent was removed under reduced pressure. The crude product was further purified through a Prep-HPLC system to generate the product as a white solid (20.5 mg, 38%). HRMS (ESI−) calculated for C₃₇H₆₁N₉O₂₂P₂S (M−H⁺) 1076.3054, found 1076.3068. ¹H NMR (400 MHz, D₂O) δ 8.12 (s, 1H), 5.92 (d, J=6.1 Hz, 1H), 4.93 (t, J=7.8 Hz, 1H), 4.78-4.77 (m, 1H), 4.59 (dd, J=7.9, 4.6 Hz, 1H), 4.53 (dd, J=5.2, 3.4 Hz, 1H), 4.39 (dd, J=7.9, 4.4 Hz, 1H), 4.35-4.34 (m, 1H), 4.22 (dd, J=5.4, 3.4 Hz, 2H), 3.87 (d, J=3.1 Hz, 1H), 3.76 (t, J=6.3 Hz, 2H), 3.69-3.66 (m, 14H), 3.63-3.60 (m, 3H), 3.59-3.56 (m, 1H), 3.38 (t, J=5.3 Hz, 2H), 3.32-3.26 (m, 2H), 2.97 (dd, J=13.1, 5.0 Hz, 1H), 2.77 (d, J=13.0 Hz, 1H), 2.56 (t, J=6.2 Hz, 2H), 2.26 (t, J=7.3 Hz, 2H), 1.74-1.51 (m, 4H), 1.42-1.34 (m, 2H).

### Example 18 Synthesis of GDP-FAzP₄Biotin

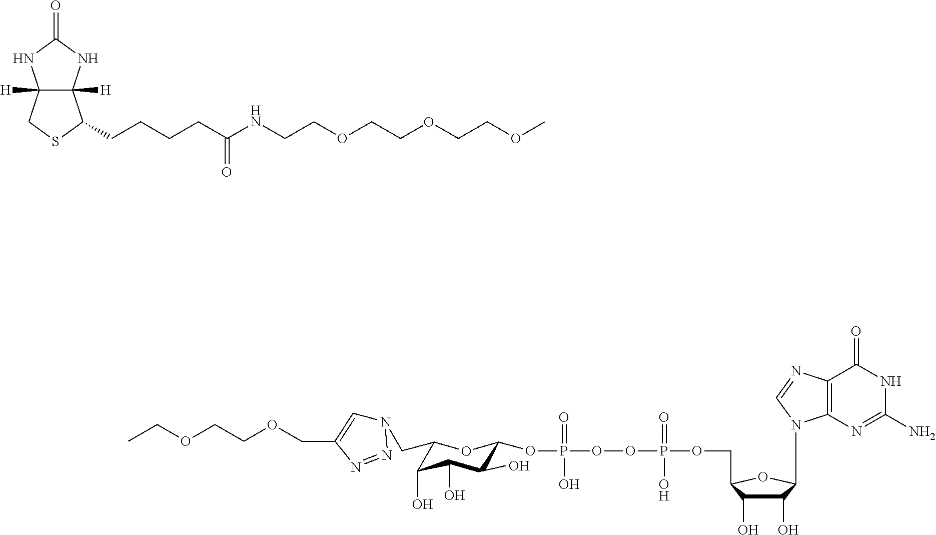

400 μL GDP-FAz (50 mM in ddH₂O), 400 μL CuSO₄/BTTP (5 mM/10 mM in ddH₂O), 210 μL propargyl-PEG₄-Biotin (Click Chemistry Tools) (100 mM in MeOH), 40 μL ascorbate sodium (250 mM in ddH₂O) and 2.95 mL ddH₂O were mixed together. The reaction was allowed for stirring at r.t. for 5 h and monitored by TLC. Then, 2 mM BCS (bathocuproine sulphonate) was added to quench the reaction and the solvent was removed under reduced pressure. The crude product was further purified through a Prep-HPLC system to generate the product as a white solid. (14.4 mg, yield 66%). HRMS (ESI−) calculated for C₃₇H₅₉N₁₁O₂₁P₂S (M−H⁺) 1086.3010, found 1086.3040. ¹H NMR (400 MHz, D₂O) δ 8.15 (s, 1H), 8.11 (s, 1H), 5.89 (d, J=6.0 Hz, 1H), 4.94-4.90 (m, 1H), 4.77-4.76 (m, 1H), 4.73-4.68 (m, 1H), 4.65 (d, J=3.1 Hz, 2H), 4.62-4.59 (m, 1H), 4.58-4.56 (m, 1H), 4.53-4.51 (m, 1H), 4.38 (dd, J=8.0, 4.4 Hz, 1H), 4.34-4.31 (m, 1H), 4.25-4.16 (m, 2H), 4.05-4.02 (m, 1H), 3.82 (s, 1H), 3.72-3.65 (m, 14H), 3.61 (t, J=5.3 Hz, 2H), 3.37 (t, J=5.2 Hz, 2H), 3.30-3.25 (m, 1H), 2.96 (dd, J=13.1, 5.0 Hz, 1H), 2.77-2.72 (m, 1H), 2.24 (t, J=7.3 Hz, 2H), 1.74-1.49 (m, 4H), 1.40-1.32 (m, 2H).

### Example 19 Generation of Trastuzumab-G2F Using Bovine β(1,4)-GalT1(Y289L)

Trastuzumab (10 mg/mL) was incubated with UDP-galactose (5 mM) and bovine β(1,4)-GalT1(Y289L)(SEQ ID NO: 1) (0.5 mg/mL) in 25 mM Tris-HCl buffer (pH 8.0) with 10 mM MnCl₂ at 30° C. for overnight. The modified trastuzumab was purified with protein A resin. Mass spectral analysis showed the formation of one major peak (found as 148713 Da, >90%).

The amino acid sequence of the heavy chain of trastuzumab is as set forth in SEQ ID NO: 9 the amino acid sequence of the light chain of trastuzumab is as set forth in SEQ ID NO: 8.

### Example 20 Preparation of Trastuzumab-G2F-Fuc*

Trastuzumab-G2F (5 mg/mL) was incubated with GDP-Fuc* (GDP-FD1, GDP-FD2 or GDP-FD4)(5 mM) and HpFT-2 (SEQ ID NO: 19) (0.7 mg/mL) in 50 mM Tris-HCl buffer (pH 7.5) with 20 mM MgCl₂ at 30° C. for 48 h. The reaction mixture was purified with protein A resin to generate the trastuzumab-G₂F-Fuc* conjugates. Mass spectral analysis showed the formation of one major peak corresponding to trastuzumab-G₂F-FD1 (found as 152228 Da, MAR 4+4), trastuzumab-G₂F-FD2 (found as 151648 Da, MAR 4+4) and trastuzumab-G₂F-FD4 (found as 155376 Da, MAR 4+4) (FIG. 4A-4C) respectively. All the compositions of protein conjugates have average MARs of 3.2-4.0 and 3.2-4.0.

### Example 21 Preparation of Trastuzumab-G2F-FD5

Trastuzumab-G2F (5 mg/mL) was incubated with GDP-FD5 (5 mM) and HpFT-2 (1.0 mg/mL) in 50 mM Tris-HCl buffer (pH 7.5) with 20 mM MgCl₂ at 30° C. for 72 h. The reaction mixture was purified with protein A resin to generate the trastuzumab-G₂F-Fuc* conjugates. Mass spectral analysis showed the formation of one major peak corresponding to trastuzumab-G₂F-FD5 (found as 159708 Da, MAR 4+4, >90%). The composition of protein conjugates have average MARs of 3.2-4.0 and 3.2-4.0.

### Example 22 Preparation of Antibody-GlcNAc

Antibodies (e.g., trastuzumab, hRS7) (10 mg/mL) were incubated with EndoS (SEQ ID NO: 3) (0.05 mg/mL) and Alfe (SEQ ID NO: 5) (1 mg/mL) in 50 mM Tris-HCl buffer (pH 7.5) at 37° C. for 24 h. The reaction mixture was purified with protein A resin to generate the antibody-GlcNAc. Mass spectral analysis showed the complete conversion to trastuzumab-GlcNAc (found as 145582 Da), hRS7-GlcNAc (found as 145426 Da), respectively.

The amino acid sequence of the heavy chain of hRS7 is as set forth in SEQ ID NO: 11, the amino acid sequence of the light chain of hRS7 is as set forth in SEQ ID NO: 10.

### Example 23 Preparation of Antibody-(GalXβ1,4)GlcNAc

The antibody-GlcNAc (10 mg/mL) (e.g., as prepared in Example 22) was incubated with UDP-GalX (UDP-Gal, UDP-GalNAc, UDP-GalNAz or UDP-GalNH₂) (5 mM) and bovine β1,4-GalT1 (Y289L) (0.3 mg/mL) in 50 mM Tris-HCl buffer (pH 7.5) with 10 mM MnCl₂ for overnight to 72 h at 30° C. The reaction mixture was purified with protein A resin to generate the product. Mass spectral analysis showed the complete conversion to trastuzumab-(Galβ1,4)GlcNAc (found as 145907 Da), trastuzumab-(GalNAcβ1,4)GlcNAc (found as 145986 Da), trastuzumab-(GalNAzβ1,4)GlcNAc (found as 146070 Da), hRS7-(Galβ1,4)GlcNAc (found as 145750 Da), hRS7-(GalNH₂β1,4)GlcNAc (found as 145747 Da) and hRS7-(GalNAcβ1,4)GlcNAc (found as 145830 Da), respectively.

### Example 24 Preparation of Trastuzumab-(Fucα1,6)(GalXβ1,4)GlcNAc

Trastuzumab (8 mg/mL) were incubated with EndoS (0.05 mg/mL) and bovine β1,4-GalT₁(Y289L) (0.3 mg/mL) and UDP-Galactose or UDP-GalNAz (5 mM) in 50 mM Tris-HCl buffer (pH 7.5) with 10 mM MnCl₂ for overnight to 72 h at 30° C. The reaction mixture was purified with protein A resin to generate the product. Mass spectral analysis showed the complete conversion to trastuzumab-(Fucα1,6)(Galβ1,4)GlcNAc (found as 146192 Da) and trastuzumab-(Fucα1,6)(GalNAzβ1,4)GlcNAc (found as 146357 Da) respectively.

### Example 25 Preparation of Antibody-(Galβ1,4)GlcNAc-Fuc*

Trastuzumab-(Galβ1,4)GlcNAc or hRS7-(Galβ1,4)GlcNAc (8 mg/mL) was incubated with GDP-Fuc* (GDP-FD1, GDP-FD2, GDP-FD3, GDP-FD4, GDP-FD5, GDP-FD6, GDP-FD7, GDP-FD8 or GDP-FD9) (5 mM) and HpFT-2 (0.5 mg/mL) in 50 mM Tris-HCl buffer (pH 7.5) with 20 mM MgCl₂ at 30° C. for 24 h to 48 h. The reaction mixture was purified with protein A resin to generate the antibody-(Galβ1,4)GlcNAc-Fuc* conjugates. Mass spectral analysis showed the formation of one major peak corresponding to trastuzumab-(Galβ1,4)GlcNAc-FD1 (found as 147659 Da, MAR 2+2), trastuzumab-(Galβ1,4)GlcNAc-FD3 (found as 147598 Da, MAR 2+2), trastuzumab-(Galβ1,4)GlcNAc-FD4 (found as 149236 Da, MAR 2+2), trastuzumab-(Galβ1,4)GlcNAc-FD7 (found as 152834 Da, MAR 2+2), trastuzumab-(Galβ1,4)GlcNAc-FD8 (found as 151704 Da, MAR 2+2), hRS7-(Galβ1,4)GlcNAc-FD1 (found as 147511 Da, MAR 2+2), hRS7-(Galβ1,4)GlcNAc-FD2 (found as 147221 Da, MAR 2+2), hRS7-(Galβ1,4)GlcNAc-FD3 (found as 147455 Da, MAR 2+2), hRS7-(Galβ1,4)GlcNAc-FD4 (found as 149084 Da, MAR 2+2), hRS7-(Galβ1,4)GlcNAc-FD5 (found as 151251 Da, MAR 2+2), hRS7-(Galβ1,4)GlcNAc-FD6 (found as 151819 Da, MAR 2+2), hRS7-(Galβ1,4)GlcNAc-FD8 (found as 151553 Da, MAR 2+2) and hRS7-(Galβ1,4)GlcNAc-FD9 (found as 150193 Da, MAR 2+2), respectively. (FIG. 5A-5M). All the compositions of protein conjugates have average MARs of 1.6-2.0 and 1.6-2.0.

In terms of trastuzumab-(Galβ1,4)GlcNAc-FD4, mass spectral analysis showed the formation of a major peak corresponding to the trastuzumab-(Galβ1,4)GlcNAc-FD4 and a minor peak (found as 148475 Da) due to fragmentation of the vc-PAB linker during mass spectrometry, similar fragments appeared in the following antibody-drug conjugates which containing the vc-PAB linkers Example 26 Preparation of trastuzumab-(Fucα1,6)(Galβ1,4)GlcNAc-FD1 Trastuzumab-(Fucα1,6)(Galβ1,4)GlcNAc (8 mg/mL) was incubated with GDP-FD1 (5 mM) and HpFT-2 (0.5 mg/mL) in 50 mM Tris-HCl buffer (pH 7.5) with 20 mM MgCl₂ at 30° C. for 48 h. The reaction mixture was purified with protein A resin to generate the trastuzumab-(Fucα1,6)(Galβ1,4)GlcNAc-FD1 conjugates. Mass spectral analysis showed the formation of one major peak corresponding to trastuzumab-(Fucα1,6)(Galβ1,4)GlcNAc-FD1 (found as 147948 Da, MAR 2+2). The composition of protein conjugates have average MARs of 1.6-2.0 and 1.6-2.0.

### Example 27 Preparation of Antibody-(GalNAzβ1,4)GlcNAc-Fuc*

Trastuzumab-(GalNAzβ1,4)GlcNAc (6 mg/mL) was incubated with GDP-Fuc* (GDP-FD1 or GDP-FD2) (5 mM) and HpFT-2 (0.5 mg/mL) in 50 mM Tris-HCl buffer (pH 7.5) with 20 mM MgCl₂ at 30° C. for 40 h. The reaction mixture was purified with protein A resin to generate the trastuzumab-(GalNAzβ1,4)GlcNAc-Fuc* conjugates. Mass spectral analysis showed the formation of one major peak corresponding to trastuzumab-(GalNAzβ1,4)GlcNAc-FD1 (found as 147825 Da, MAR 2+2), and trastuzumab-(GalNAzβ1,4)GlcNAc-FD2 (found as 147535 Da, MAR 2+2), respectively (FIG. 5N-50). All the compositions of protein conjugates have average MARs of 1.6-2.0 and 1.6-2.0.

### Example 28 Preparation of Trastuzumab-(GalNAzβ1,4)GlcNAc-FD5

Trastuzumab-(GalNAzβ1,4)GlcNAc (6 mg/mL) was incubated with GDP-FD5 (5 mM) and HpFT-2 (0.5 mg/mL) in 50 mM Tris-HCl buffer (pH 7.5) with 20 mM MgCl₂ at 30° C. for 48 h. The reaction mixture was purified with protein A resin to generate the trastuzumab-(GalNAzβ1,4)GlcNAc-FD5 conjugates. Mass spectral analysis showed the formation of one major peak corresponding to trastuzumab-(GalNAzβ1,4)GlcNAc-FD5 (found as 151564 Da, MAR 2+2, >90%). The composition of protein conjugates have an average MARs of 1.6-2.0 and 1.6-2.0.

### Example 29 Preparation of Antibody-(GalNAcβ1,4)GlcNAc-Fuc*

Trastuzumab-(GalNAcβ1,4)GlcNAc or hRS7-(GalNAcβ1,4)GlcNAc (6 mg/mL) was incubated with GDP-Fuc* (GDP-FD1, GDP-FD4 or GDP-FD5) (5 mM) and HpFT-2 (0.5 mg/mL) in 50 mM Tris-HCl buffer (pH 7.5) with 20 mM MgCl₂ at 30° C. for 48 h. The reaction mixture was purified with protein A resin to generate the antibody-(Ga1NAcβ1,4)GlcNAc-Fuc* conjugates. Mass spectral analysis showed the formation of one major peak corresponding to trastuzumab-(GalNAcβ1,4)GlcNAc-FD1 (found as 147743 Da, MAR 2+2), hRS7-(GalNAcβ1,4)GlcNAc-FD4 (found as 149164 Da, MAR 2+2), hRS7-(GalNAcβ1,4)GlcNAc-FD5 (found as 151331 Da, MAR 2+2) (FIGS. 5P, 5Q and 5S).

### Example 30 Preparation of Antibody-(GalNH2β1,4)GlcNAc-Fuc*

hRS7-(GalNH₂β_{1,4})GlcNAc (6 mg/mL) was incubated with GDP-Fuc* (GDP-FD4 or GDP-FD5) (5 mM) and HpFT-2 (0.5 mg/mL) in 50 mM Tris-HCl buffer (pH 7.5) with 20 mM MgCl₂ at 30° C. for 40 h. The reaction mixture was purified with protein A resin to generate the hRS7-(GalNH₂β_{1,4})GlcNAc-Fuc* conjugates. Mass spectral analysis showed the formation of one major peak corresponding to hRS7-(GalNH₂β1,4)GlcNAc-FD4 (found as 149080 Da, MAR 2+2), hRS7-(GalNH₂β1,4)GlcNAc-FD5 (found as 151248 Da, MAR 2+2) respectively (FIGS. 5R and 5T).

### Example 31 Preparation of Antibody-(Fucα1,6)(GalNAzβ1,4)GlcNAc-FD1

Trastuzumab-(Fucα1,6)(GalNAzβ1,4)GlcNAc (6 mg/mL) was incubated with GDP-FD1 (5 mM) and HpFT-2 (0.5 mg/mL) in 50 mM Tris-HCl buffer (pH 7.5) with 20 mM MgCl₂ at 30° C. for 72 h. The reaction mixture was purified with protein A resin to generate the trastuzumab-(Fucα1,6)(GalNAzβ1,4)GlcNAc-FD1 conjugates. Mass spectral analysis showed the formation of one major peak corresponding to trastuzumab-(Fucα1,6)(GalNAzβ1,4)GlcNAc-FD1 (found as 148116 Da, MAR 2+2) (FIG. 5U).

### Example 32 Comparison of the Conversion Efficiency of α1,3 Fucosyltrasferase Towards GDP-Fucose Derivatives with Different Jointer on the Antibodies

Trastuzumab-(Galβ1,4)GlcNAc (2 mg/mL) was incubated with GDP-FAzP₄Biotin (1 mM) or GDP-FAmP₄Biotin (1 mM) and HpFT-2 (0.5 mg/mL) in 50 mM Tris-HCl buffer (pH 7.5) with 5 mM MgCl₂ at 30° C. for 10 minutes. Trastuzumab-(Fucα1,6)(Galβ1,4)GlcNAc (2 mg/mL) was incubated with GDP-FAzP₄Biotin (1 mM) or GDP-FAmP₄Biotin (1 mM) and HpFT-2 (0.5 mg/mL) in 50 mM Tris-HCl buffer (pH 7.5) with 5 mM MgCl₂ at 30° C. for 10 minutes. Trastuzumab-(GalNAzβ1,4)GlcNAc (2 mg/mL) was incubated with GDP-FAzP₄Biotin (1 mM) or GDP-FAmP₄Biotin (1 mM) and HpFT-2 (0.5 mg/mL) in 50 mM Tris-HCl buffer (pH 7.5) with 5 mM MgCl2 at 30° C. for 2 hours. Trastuzumab-G₂F (2 mg/mL) was incubated with GDP-FAzP₄Biotin (1 mM) or GDP-FAmP₄Biotin (1 mM) and HpFT-2 (0.5 mg/mL) in 50 mM Tris-HCl buffer (pH 7.5) with 5 mM MgCl₂ at 30° C. for 2 hours. The reaction mixtures were then quenched by adding LacNAc (final concentration 10 mM) and purified with protein A resin and analyzed by LC-MS respectively. The results were listed in the table below. For trastuzumab-G₂F, % of conversion=average MAR/4*100%. For trastuzumab-(Fucα1,6)(Galβ1,4)GlcNAc, trastuzumab-(Galβ1,4)GlcNAc, and trastuzumab-(GalNAzβ1,4)GlcNAc, % of conversion=average MAR/2*100%.

The results showed that the α1,3 fucosyltrasferase (e.g. Helicobacter pylori α1,3 fucosyltrasferase) displayed significantly higher conversion efficiency towards the GDP-fucose derivatives with a jointer of

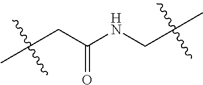 [SMILES: *CNC(=O)C*]

than those with a jointer of

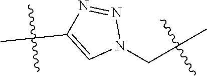 [SMILES: *Cn1cc(*)nn1]

on the antibodies. Especially for the trastuzumab-G₂F, the conversion efficiency of GDP-FAmP₄Biotin were strikingly higher than that of the GDP-FAzP₄Biotin.

| | | | | % of |
|---|---|---|---|---|
| | | Reaction | | con- |
| Antibody | Substrates | time | | version |
| Trastuzumab-G₂F | GDP-FAmP₄Biotin | 2 | h | 31 ± 4.6 |
| | GDP-FAzP₄Biotin | | | 9 ± 1.2 |
| Trastuzumab- | GDP-FAmP₄Biotin | 10 | min | 83 ± 1.2 |
| (Galβ1,4)GlcNAc | GDP-FAzP₄Biotin | | | 60 ± 0.6 |
| Trastuzumab- | GDP-FAmP₄Biotin | 10 | min | 42 ± 5.3 |
| (Fucα1,6)(Galβ1,4)GlcNAc | GDP-FAzP₄Biotin | | | 30 ± 4.6 |
| Trastuzumab- | GDP-FAmP₄Biotin | 2 | h | 77 ± 5.0 |
| (GalNAzβ1,4)GlcNAc | GDP-FAzP₄Biotin | | | 13 ± 4.7 |

### Example 33 Comparison of the Conversion Efficiency of Helicobacter pylori α1,3 Fucosyltrasferase and Human α1,3 Fucosyltrasferase Towards GDP-Fuc* Bearing Two Active Molecules on Antibody-G2F, Antibody-(Galβ1,4)GlcNAc, Antibody-(Fucα1,6)(Galβ1,4)GlcNAc and Antibody-(GalNAzβ1,4)GlcNAc

Trastuzumab-G₂F (2 mg/mL) was incubated with GDP-FD1 (1 mM) and HpFT-2 (SEQ ID NO: 19) (0.1 mg/mL) or HFT6 (SEQ ID NO: 7) (0.1 mg/mL) in 50 mM Tris-HCl buffer (pH 7.5) with 5 mM MgCl₂ at 30° C. for 6 h or 24 h. Trastuzumab-(Galβ1,4)GlcNAc (2 mg/mL) was incubated with GDP-FD1 (1 mM) and HpFT-1 (SEQ ID NO: 17)(0.1 mg/mL), or HpFT-2 (0.1 mg/mL, or HFT6 (0.1 mg/mL) in 50 mM Tris-HCl buffer (pH 7.5) with 5 mM MgCl₂ at 30° C. for 1 h or 6 h. Trastuzumab-(GalNAzβ1,4)GlcNAc (2 mg/mL) was incubated with GDP-FD1 (1 mM) and HpFT-1 (0.1 mg/mL) or HpFT-2 (0.1 mg/mL), or HFT6 (0.1 mg/mL) in 50 mM Tris-HCl buffer (pH 7.5) with 5 mM MgCl₂ at 30° C. for 6 h. Trastuzumab-(Fucα1,6)(Galβ1,4)GlcNAc (2 mg/mL) was incubated with GDP-FD1 (1 mM) and HpFT-2 (0.1 mg/mL) or HFT6 (0.1 mg/mL) in 50 mM Tris-HCl buffer (pH 7.5) with 5 mM MgCl₂ at 30° C. for 1 h. The reaction mixtures were quenched by adding LacNAc (final concentration 10 mM) and then purified with protein A resin and analyzed by LC-MS respectively. The results were list in the table below. For trastuzumab-G₂F, % of conversion=average D1-to-antibody-ratio/4*100%. For trastuzumab-(Fuc1,6)(Galβ1,4)GcNAc, trastuzumab-(Galβ1,4)GlcNAc, and trastuzumab-(GaANAzβ1,4)GlcNAc, % of conversion=average D-to-antibody-ratio/2*100%. UD means “the conversion product were undetectable through the MS analysis”, indicating a very low efficiency.

| | | Reaction | | % of |
|---|---|---|---|---|
| Antibody | Enzymes | time | | conversion |
| Trastuzumab-G₂F | HpFT-2 | 6 | h | 3 ± 2.1 |
| | | 24 | h | 58 ± 1.2 |
| | HFT6 | 6 | h | UD |
| | | 24 | h | UD |
| Trastuzumab- | HpFT-2 | 1 | h | 70 ± 9.5 |
| (Galβ1,4)GlcNAc | | 6 | h | 95 ± 4.0 |
| | HpFT-1 | 1 | h | 78 ± 4.7 |
| | | 1 | h | UD |
| | HFT6 | 6 | h | UD |
| Trastuzumab- | HpFT-2 | 1 | h | 20 ± 3.2 |
| (Fucα1,6)(Galβ1,4)GlcNAc | HFT6 | 1 | h | UD |
| Trastuzumab- | HpFT-2 | 6 | h | 59 ± 2.6 |
| (GalNAzβ1,4)GlcNAc | HpFT-1 | 6 | h | 64 ± 6.4 |
| | HFT6 | 6 | h | UD |

The results showed that the human α1,3 fucosyltrasferase HFT6 showed undetectable conversion towards GDP-FD1 on all the antibodies. In contrast, the Helicobacter pylori α1,3 fucosyltrasferases showed good conversion efficiencies on all the antibodies.

In another aspect, the antibody with the -GlcNAc-Gal directly linked to the N297 of Fc domain showed strikingly higher conversion efficiency compared to the -GlcNAc-Gal linked to a mannose of an oligosaccharide linked to the N297 of Fc domain. For example, the trastuzumab-(Galβ1,4)GlcNAc showed strikely higher conversion efficiency than the trastuzumab-G₂F. For example, by using HpFT-2, the trastuzumab-G₂F showed a conversion efficiency of 3% (average D1-to-antibody-ratio of 0.1) at 6 h, while the trastuzumab-(Galβ1,4)GlcNAc showed a much higher conversion efficiency of 70% (average D1-to-antibody-ratio of 1.4) even at 1 h.

In addition, HpFT(C169S)-2 (SEQ ID NO: 21)(0.1 mg/mL), HpFT-3 (SEQ ID NO: 23)(0.1 mg/mL), or HpFT-4 (SEQ ID NO: 25)(0.1 mg/mL) was also incubated with trastuzumab-(Galβ1,4)GlcNAc (2 mg/mL) and GDP-FD1 (1 mM) in 50 mM Tris-HCl buffer (pH 7.5) with 5 mM MgCl₂ at 30° C. for 6 h. The results showed all the enzymes had a >60% of conversion after 6 h.

### Example 34 Preparation of Trastuzumab-G2F-FD1-TCO-MMAE/DBCO-MMAF

Trastuzumab-G₂F-FD1 (4 mg/mL) was incubated with DBCO-PEG₄-vc-pAB-MMAF (Levena Biopharma) (200 μM) and TCO-PEG₄-vc-pAB-MMAE (200 μM) in PBS (pH 7.4) with 8% DMSO at r.t. for 24 h. The reaction mixture was purified with protein A resin to generate the product. Mass spectral analysis showed the formation of one major peak corresponding to trastuzumab-G₂F-FD1-TCO-MMAE/DBCO-MMAF (found as 164897 Da, MAR 4+4) with four MMAE and four MMAF added to one trastuzumab-G₂F-FD1 (FIG. 7A). The compositions of protein conjugates have average MARs of 3.2-4.0 and 3.2-4.0.

### Example 35 Preparation of Trastuzumab-G2F-FD2-DBCO-MMAE

Trastuzumab-G₂F-FD2 (4 mg/mL) was incubated with DBCO-PEG₄-vc-PAB-MMAE (Levena Biopharma) (400 μM) in PBS (pH 7.4) with 8% DMSO at r.t. for 24 h. The reaction mixture was purified with protein A resin to generate the trastuzumab-G₂F-FD2-DBCO-MMAE. Mass spectral analysis showed one major peak (found as 164913 Da, MAR 4+4) with eight MMAE added to one trastuzumab-G₂F-FD2 molecule (FIG. 7B).

### Example 36 Preparation of Trastuzumab-G2F-FD1-TCO-MMAE/DBCO-MMAE

Trastuzumab-G₂F-FD1 (4 mg/mL) was incubated with DBCO-PEG₄-vc-PAB-MMAE (200 μM) and TCO-PEG₄-vc-PAB-MMAE (200 μM) in PBS (pH 7.4) with 8% DMSO at r.t. for 24 h. The reaction mixture was purified with protein A resin to generate the trastuzumab-G₂F-FD2-MMAE. Mass spectral analysis showed one major peak (found as 164836 Da, MAR 4+4) with eight MMAE added to one trastuzumab-G₂F-FD2 molecule (FIG. 7C).

### Example 37 Preparation of Trastuzumab-(GalNAcβ1,4)GlcNAc-FD1-TCO-MMAE/DBCO-MMAE

Trastuzumab-(GalNAcβ1,4)GlcNAc-FD1 (4 mg/mL) was incubated with DBCO-PEG₄-vc-PAB-MMAE (150 μM) and TCO-PEG₄-vc-PAB-MMAE (150 μM) in PBS (pH 7.4) with 8% DMSO at r.t. for overnight. The reaction mixture was purified with protein A resin to generate the product. Mass spectral analysis showed the formation of one major peak corresponding to trastuzumab-(GalNAcβ1,4)GlcNAc-FD1-TCO-MMAE/DBCO-MMAE (found as 154047 Da, MAR 2+2) with four MMAE added to one trastuzumab-(GalNAcβ1,4)GlcNAc-FD1 molecule (FIG. 7D). The compositions of protein conjugates have average MARs of 1.6-2.0 and 1.6-2.0.

### Example 38 Preparation of Trastuzumab-(GalNAzβ1,4)GlcNAc-FD1-TCO-MMAE/DBCO-MMAE

Trastuzumab-(GalNAzβ1,4)GlcNAc-FD1 (4 mg/mL) was incubated DBCO-PEG₄-vc-PAB-MMAE (200 μM) and TCO-PEG₄-vc-PAB-MMAE (150 μM) in PBS (pH 7.4) with 8% DMSO at r.t. for overnight. The reaction mixture was purified with protein A resin to generate the product. Mass spectral analysis showed the formation of one major peak corresponding to trastuzumab-(GalNAzβ1,4)GlcNAc-FD1-TCO-MMAE/DBCO-MMAE (found as 157445 Da, MAR 2+2+2) with six MMAE added to one trastuzumab-(GalNAzβ1,4)GlcNAc-FD1 molecule (FIG. 7E).

### Example 39 Preparation of Trastuzumab-(GalNAzβ1,4)GlcNAc-FD2-DBCO-MMAE

Trastuzumab-(GalNAzβ1,4)GlcNAc-FD2 (4 mg/mL) was incubated DBCO-PEG₄-vc-PAB-MMAE (300 μM) in PBS (pH 7.4) with 8% DMSO at r.t. for overnight. The reaction mixture was purified with protein A resin to generate the product. Mass spectral analysis showed the formation of one major peak corresponding to trastuzumab-(GalNAzβ1,4)GlcNAc-FD2-MMAE/DBCO-MMAE (found as 157484 Da, MAR 2+2+2) with six MMAE added to one trastuzumab-(GalNAzβ1,4)GlcNAc-FD2 molecule (FIG. 7F).

### Example 40 Preparation of Trastuzumab-(GalNAzβ1,4)GlcNAc-FD1-TCO-MMAE/DBCO-MMAF

Trastuzumab-(GalNAzβ1,4)GlcNAc-FD1 (4 mg/mL) was incubated DBCO-PEG₄-vc-PAB-MMAF (200 μM) and TCO-PEG₄-vc-PAB-MMAE (150 μM) in PBS (pH 7.4) with 8% DMSO at r.t. for overnight. The reaction mixture was purified with protein A resin to generate the product. Mass spectral analysis showed the formation of one major peak corresponding to trastuzumab-(GalNAzβ1,4)GlcNAc-FD1-TCO-MMAE/DBCO-MMAF (found as 157505 Da, MAR 2+2+2) with two MMAE and four MMAF added to one trastuzumab-(GalNAzβ1,4)GlcNAc-FD1 molecule (FIG. 7G).

### Example 41 Preparation of Trastuzumab-(GalNAzDBCO-Seco-DUBA)GlcNAc-FD5

Trastuzumab-(GalNAzβ1,4)GlcNAc-FD5 (4 mg/mL) was incubated with DBCO-PEG₄-vc-PAB-seco-DUBA (150 μM) in PBS (pH 7.4) with 50% propylene glycol at r.t. for 24 h. The reaction mixture was purified with protein A resin to generate the product. Mass spectral analysis showed the formation of one major peak corresponding to trastuzumab-(GalNAzDBCO-seco-DUBA)GlcNAc-FD5 (found as 154871 Da, MAR 2+2+2) with two MMAE and two DXd and two seco-DUBA added to one trastuzumab-(GalNAzβ1,4)GlcNAc molecule.

### Example 42 Preparation of hRS7-(Galβ1,4)GlcNAc-FD1-TCO-MMAE/DBCO-DXd

hRS7-(Galβ1,4)GlcNAc-FD1 (4 mg/mL) was incubated with DBCO-PEG₄-GGFG-DXd (Abydos Scientific) (150 μM) and TCO-PEG₄-vc-PAB-MMAE (150 μM) in PBS (pH 7.4) with 8% DMSO at r.t. for overnight. The reaction mixture was purified with protein A resin to generate the product. Mass spectral analysis showed the formation of one major peak corresponding to hRS7-(Galβ1,4)GlcNAc-FD1-TCO-MMAE/DBCO-DXd (found as 153250 Da, MAR 2+2) with two MMAE and two DXd added to one hRS7-(Galβ1,4)GlcNAc-FD1 molecule (FIG. 7H).

### Example 43 Intact Protein Mass Analysis

For LC-MS analysis, the purified proteins were analyzed on an Xevo G2-XS QTOF MS System (Waters Corporation) equipped with an electrospray ionization (ESI) source in conjunction with Waters Acquity UPLC I-Class plus. Separation and desalting were carried out on a waters ACQUITY UPLC Protein BEH C₄ Column (300 Å, 1.7 μm, 2.1 mm×100 mm). Mobile phase A was 0.1% formic acid in water and mobile phase B was acetonitrile with 0.1% formic acid. A constant flow rate of 0.2 mL/min was used. Data were analyzed using Waters Unify software. Mass spectral deconvolution was performed using a Unify software (version 1.9.4, Waters Corporation). Some of the results were shown in FIGS. 4, 5 and 7.

### Example 44 HIC-HPLC Assay

Some antibody-drug conjugates were evaluated by HIC-HPLC analysis using the Agilent 1260 HPLC system with a TSKgel Butyl-NPR column (4.6 mm×35 mm, 2.5 μm; TOSOH; Tokyo, Japan) under the following conditions: (1) buffer A: 20 mM sodium phosphate, 1.5 M ammonium sulfate (pH 6.9); (2) buffer B: 75% (v/v) 20 mM sodium phosphate, 25% (v/v) isopropanol (pH 6.9); (3) flow rate: 0.4 mL/min; (4) gradient: from 100% buffer A to 100% buffer B (over 1-16 min); and (5) column temperature was 30° C. HIC-HPLC analysis showed the high homogeneity of trastuzumab drug conjugates and hRS7 drug conjugates (FIG. 8).

### Example 45 Binding Assay of Antibody-Drug Conjugates Towards its Antigen

Recombinant Her2 extracellular domains (HER2, novoprotein) was diluted to a final concentration of 250 ng/mL with coating buffer and plated on 96-well plates (100 μL/well) at 4° C. for overnight. After removing the coating solution, the plates were blocked with 3% (v/v) bovine serum albumin in PBS for 2 h at 37° C. After washing with PBST (PBS containing 0.03% tween-20) for 3 times, trastuzumab, trastuzumab-(Galβ1,4)GlcNAc-FD8 (2 DXd+2 DXd) and trastuzumab-(GalNAzβ1,4)GlcNAc-FD5 (2 MMAE+2 DXd) were added to PBST (with 1% (v/v) bovine serum albumin in PBS) to a series of final concentrations (1000 ng/mL, 333.33 ng/mL, 111.11 ng/mL, 37.04 ng/mL, 12.35 ng/mL, 4.12 ng/mL, 1.37 ng/mL, 0.46 ng/mL, 0.15 ng/mL, 0 ng/mL) and added to the plates respectively. After incubating for 1.5 h, the plates were washed 3 times with PBST, then horseradish peroxidase (HRP)-conjugated donkey anti-human IgG antibody was added to each well and incubated for 1 h at 37° C. Finally, each well was washed with PBST for 3 times, and then tetramethyl benzidine substrate was cotreated to produce color for visualization. The reaction in each well was stopped by adding 100 μL of 3 M HCl after 15 min of incubation. The absorbance was read at 450 nm on a Synergy™ LX plate reader. The result showed a similar HER2-binding affinity between trastuzumab and trastuzumab conjugates (FIG. 9).

### Example 46 In Vitro Plasma Stability Assay

Human plasma was co-treated with protein A resin for 1 h at r.t. then centrifuged at 200 g for 5 min to removal the IgG. The depleted IgG plasma was filter sterilized by 0.2 μM filter. The hRS7-(Galβ1,4)GlcNAc-FD5 (2 MMAE+2 DXd) was incubated with the plasma to a final concentration of 100 μg/mL at 37° C. and 5% CO₂ in an incubator. Samples were taken at 0, 2, 4, 8 days and purified with protein A followed by MS analysis. Mass spectral analysis showed the peak corresponding to the hRS7-(Galβ1,4)GlcNAc-FD5 did not decrease in time. Meanwhile, no degradation peaks could be detected, indicating that the sample was stable in human plasma for at least 8 days (FIG. 10) Example 47 In vitro efficacy of hRS7-drug-conjugates JIMT-1 (Trop2 high expression) and MDA-MB-231 (Trop2 low expression) cells were cultured in DMEM (Gibco) supplemented with 10% FBS (Gibco). The cells were plated in 96-well plates with 5000 cells per well and were incubated for 24 hours at 37° C. and 5% CO₂. After removing of the culture medium, hRS7, hRS7-(Galβ1,4)GlcNAc-FD6 (2 MMAE+2 MMAE), hRS7-(Galβ1,4)GlcNAc-FD8 (2 DXd+2 Dxd) and hRS7-(Galβ1,4)GlcNAc-FD5 (2 MMAE+2 DXd) were added to the culturing medium to a series of final concentrations (10 nM, 1 nM, 0.5 nM, 0.1 nM, 0.05 nM, 0.01 nM, 0.001 nM and 0 nM) and added to the plates respectively. The cells were incubated for 6 days at 37° C. and 5% CO₂ and subjected to a CellTiter-Glo® Luminescent Cell Viability Assay (Promega) to measure the cell viability. The dual-drug conjugate hRS7-(Galβ1,4)GlcNAc-FD5 showed similar efficacy towards JIMT-1 cells compared to the MMAE-conjugate hRS7-(Galβ1,4)GlcNAc-FD6, while showed higher efficacy compared to the DXd-conjugate hRS7-(Galβ1,4)GlcNAc-FD8 (FIG. 11).

### Example 48 In Vivo Efficacy of hRS7-Drug-Conjugates on a Nude Mouse Human Breast Cancer JIMT-1 Xenograft Model

Female BALB/c nude mice (4-5-week-old) were inoculated with 1×10⁶ JIMT-1 (trop2 high expression) cells which were resuspended in 50% PBS (pH7.4) and 50% matrigel (BD). When the average tumor size reached 100-200 mm³, the PBS, hRS7 (5 mg/kg), hRS7-(Galβ1,4)GlcNAc-FD6 (2 MMAE+2 MMAE, 5 mg/kg), hRS7-(Galβ1,4)GlcNAc-FD8 (2 DXd+2 Dxd, 5 mg/kg) and hRS7-(Galβ1,4)GlcNAc-FD5 (2 MMAE+2 DXd, 5 mg/kg) were injected to different groups (n=6 mice per group) through the tail vein for one time respectively. The total length of the animal study was 28 days, and the tumor size and body weight of the mice were monitored twice per week throughout the study period. Tumor volumes were determined according to the formula: tumor volume (mm³)=π×long diameterx(short diameter)²/6. The DAR 2+2 dual-drug conjugate hRS7-(Galβ1,4)GlcNAc-FD5 showed similar efficacy of inhibiting tumor growth towards JIMT-1 tumor compared to the DAR4 MMAE conjugate hRS7-(Galβ1,4)GlcNAc-FD6, while showed higher efficacy compared to the DAR 4 DXd conjugate hRS7-(Galβ1,4)GlcNAc-FD8 (FIG. 12). All animal studies were conducted in accordance with Institutional Animal Care and Use Committee guidelines and were performed at Hangzhou Medical College.

### Example 49 Cloning, Expression and Purification of BGalT1(Y289L) (Bovine β-1,4-Galactosyltransferase I), EndoS (Streptococcus pyogenes Endoglycosidase S), Alfc (Lactobacillus casei α-1,6-Fucosidase Alfc) and HFT6 (Human Fucosyltransferase-6)

The cloning, expression and purification of BGalT1(Y289L) (SEQ ID NO: 1), EndoS (SEQ ID NO: 3), AlfC (SEQ ID NO: 5) and HFT6 (SEQ ID NO: 7) were performed according to the reported procedure by Qasba, P. K et al. (Prot. Expr. Fur. 2003, 30, 219) (J. Biol. Chem. 2002, 277, 20833.), by Collin, M. et al. (EMBO J. 2001, 20, 3046; Infect. Immun. 2001, 69, 7187), by Wang L., et al. (Methods Mol. Biol. 2018, 19, 367), and by Moremen K. W et al. (Nat Chem. Biol. 2018, 14, 156), respectively.

### Example 50 Cloning, Expression and Purification of Helicobacter pylori α1,3 Fucosyltrasferase

Genes encoding the Helicobacter pylori α1,3 fucosyltrasferase (amino acid sequence of SEQ ID NO: 17 (HpFT-1), SEQ ID NO: 19 (HpFT-2), SEQ ID NO:21 (HpFT(C169S)-2), SEQ ID NO: 23 (HpFT-3), SEQ ID NO:25 (HpFT-4)) were synthesized and subcloned into a pET24b vector at NdeI and BamHI by Genscript. E. coli BL21(DE3) transformed with the plasmids were cultured at 37° C. in LB with 50 μg/mL kanamycin until OD600=0.6-0.8. IPTG was added to a final concentration of 0.2 mM and protein expression was induced for sixteen hours at 25° C. The cells were harvested by centrifugation and resuspended in lysis buffer (25 mM Tris pH 7.5, 500 mM NaCl, 20 mM imidazole and 1 mM PMSF). Cells were lysed by sonication and the clarified supernatant was purified on Ni-NTA agarose (GE Health) following the manufacturer's instructions. Fractions that were >90% purity, as judged by SDS-PAGE, were consolidated and dialyzed against Tris-buffered saline (25 mM Tris pH 7.5, 150 mM NaCl).

### Example 51 Cloning, Expression and Purification of hRS7

The sequence of hRS7 antibody light chain and heavy chain were referenced to the patent (U.S. Pat. No. 7,238,785 B2). The gene encoding the light chain and the heavy chain of hRS7 were synthesized and clone into a PPT5 vector respectively by Genescript. Then, FreeStyle 293F cells were grown to a density of ˜2.5×10⁶ cells/ml and transfected by direct addition of 0.37 g/ml and 0.66 g/ml of the light chain and heavy chain expression plasmid DNA, and 2.2 g/ml polyethylenimine (linear 25 kDa PEI, Polysciences, Inc, Warrington, PA) to the suspension cultures. The cultures were diluted 1:1 with Freestyle 293 expression medium containing 4.4 mM valproic acid (2.2 mM final) 24 h after transfection, and protein production was continued for another 4-5 d at 37° C. After protein production, the antibodies were purified through the protein A agarose following the manufacturer's instructions.

While exemplary embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A protein conjugate, which comprises a protein and an oligosaccharide comprising a structure of Formula (I): 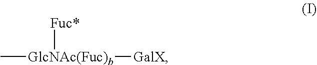
wherein:
said GlcNAc is directly or indirectly linked to an amino acid of said protein, and said protein comprises an antigen binding and/or an Fc fragment:
said GalX is an optionally substituted galactose,
said Fuc is a fucose, and b is 0 or 1,
said Fuc* comprises the structure of Formula (II): 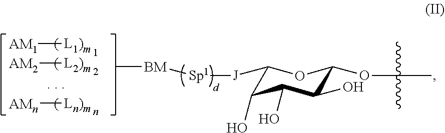
wherein
J has a structure of 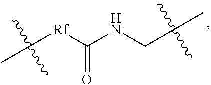
wherein R_{f} is —CH₂—, —NH— or —O—, and the right side of the structure of 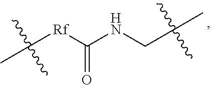
is directly linked to the 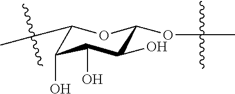
of Formula (II):
Sp¹ is selected from the group consisting of: 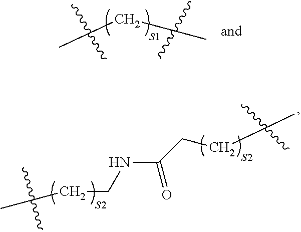
said S1 is an integer from 1-50, each said S2 is independently an integer from 0-50, each said —CH₂— is independently optionally replaced by —O— with the proviso that two or more consecutive —CH₂— are not simultaneously replaced by —O—, the right side of the structure is linked to said J and the left side of the structure is linked to said BM, and d is 0 or 1;
BM comprises 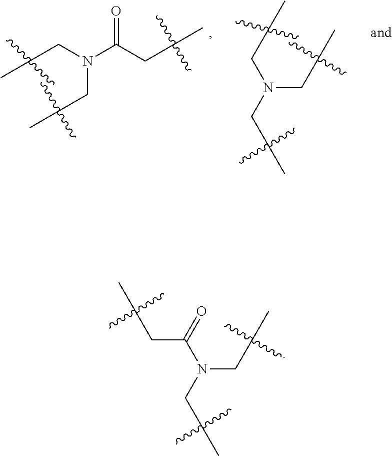 [SMILES: *CC(=O)N(C*)C* ; *CN(C*)C* ; *CC(=O)N(C*)C*]
each of L₁ to Lₙ is independently a linker of Formula (IV): (CL)ᵥ-(FL)ₓ (IV), wherein
FL is a spacer moiety selected from the group consisting of: 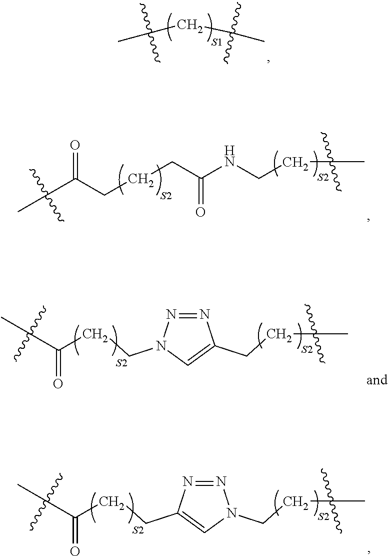
wherein said S1 is an integer from 1-50, each said S2 is independently an integer from 0-50, each said —CH₂— is independently optionally replaced by —O— with the proviso that two or more consecutive —CH₂— are not simultaneously replaced by —O—, the right side of the structure is linked to said BM, and the left side of the structure is linked to said CL or each of said AM₁ to AMₙ, x is 0 or 1;
CL is a cleavable linker, and CL is an acid-labile linker, a redox-active linker, a photo-active linker and/or a proteolytically cleavable linker, and y is 0 or 1;
the right side of Formula (IV) is linked to said BM and the left side Formula (IV) is linked to said AM,
and m₁ to mₙ each independently is 0 or 1;
AM₁ to AMₙ each independently is a chemically active molecule, an enzymatically active molecule, a biologically active molecule, and/or a pharmaceutically active molecule; and
n is an integer from 2-10; and
said GalX is linked to said GlcNAc through a β1,4 linkage;
said Fuc* is linked to said GlcNAc through an α1,3 linkage; and,
n is 2, and said Fuc* comprises the structure of Formula (III):

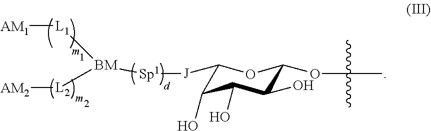

2-15. (canceled)

16. The protein conjugate of claim 1, wherein said AM₁ to AMₙ independently comprises a chemically active molecule or enzymatically active molecule X_{F}, comprises a functional moiety selected from the group consisting of 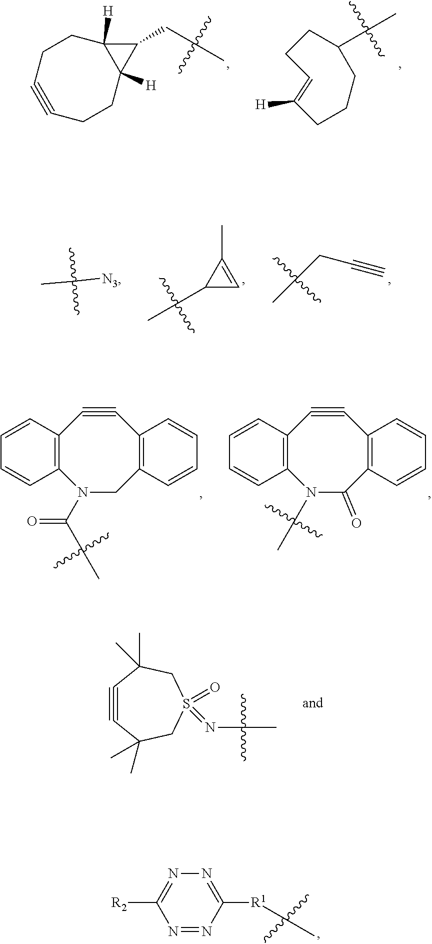
wherein R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group, and/or,
said AM₁ to AMₙ independently comprises a biologically active molecule and/or a pharmaceutically active molecule P_{F}, and P_{F} comprises a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof.

17-30. (canceled)

31. The protein conjugate of claim 1, wherein said GalX is a galactose substituted galactose and the hydroxyl group at one or more positions selected from the C2 position, the C3 position, the C4 position and the C6 position of the galactose, is substituted; and said GalX is a monosaccharide.

32-41. (canceled)

42. The protein conjugate of claim 1, wherein said GalX is selected from the group consisting of

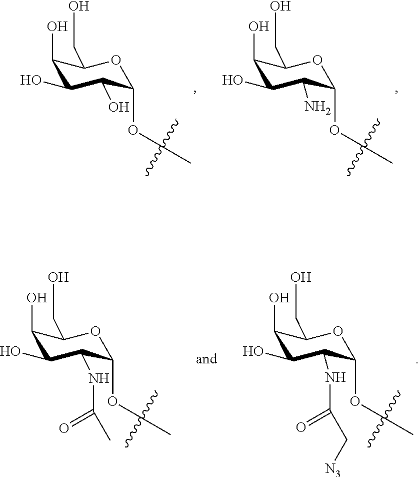

43-65. (canceled)

66. The protein conjugate of claim 1, having a structure of Formula (V) 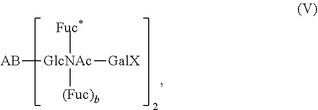
wherein AB is an antibody comprising a Fc fragment or a Fc-fusion protein, the GlcNAc is directly linked to an Asn of the Fc fragment of the AB, the Fuc is linked to the GlcNAc through an α1,6 linkage, the GalX is linked to the GlcNAc through a β1,4 linkage, the Fuc* is linked to the GlcNAc through an α1,3 linkage and b is 0 or 1.

67. (canceled)

68. The protein conjugate of claim 1, having a structure of Formula (VI): 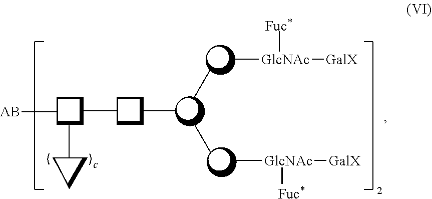
wherein AB is an antibody comprising a Fc fragment or a Fc-fusion protein, [symbol] is a GlcNAc, [symbol] is a mannose, [symbol] is a fucose linked to the [symbol] through an α1,6 linkage, c is 0 or 1;
said oligosaccharide is linked to an Asn of the Fc fragment of the AB through the [symbol] ,
the GalX is linked to the GlcNAc through a β1,4 linkage, and the Fuc* is linked to the GlcNAc through an α1,3 linkage.

69. The protein conjugate of claim 1, wherein said Fuc* is selected from the group consisting of:

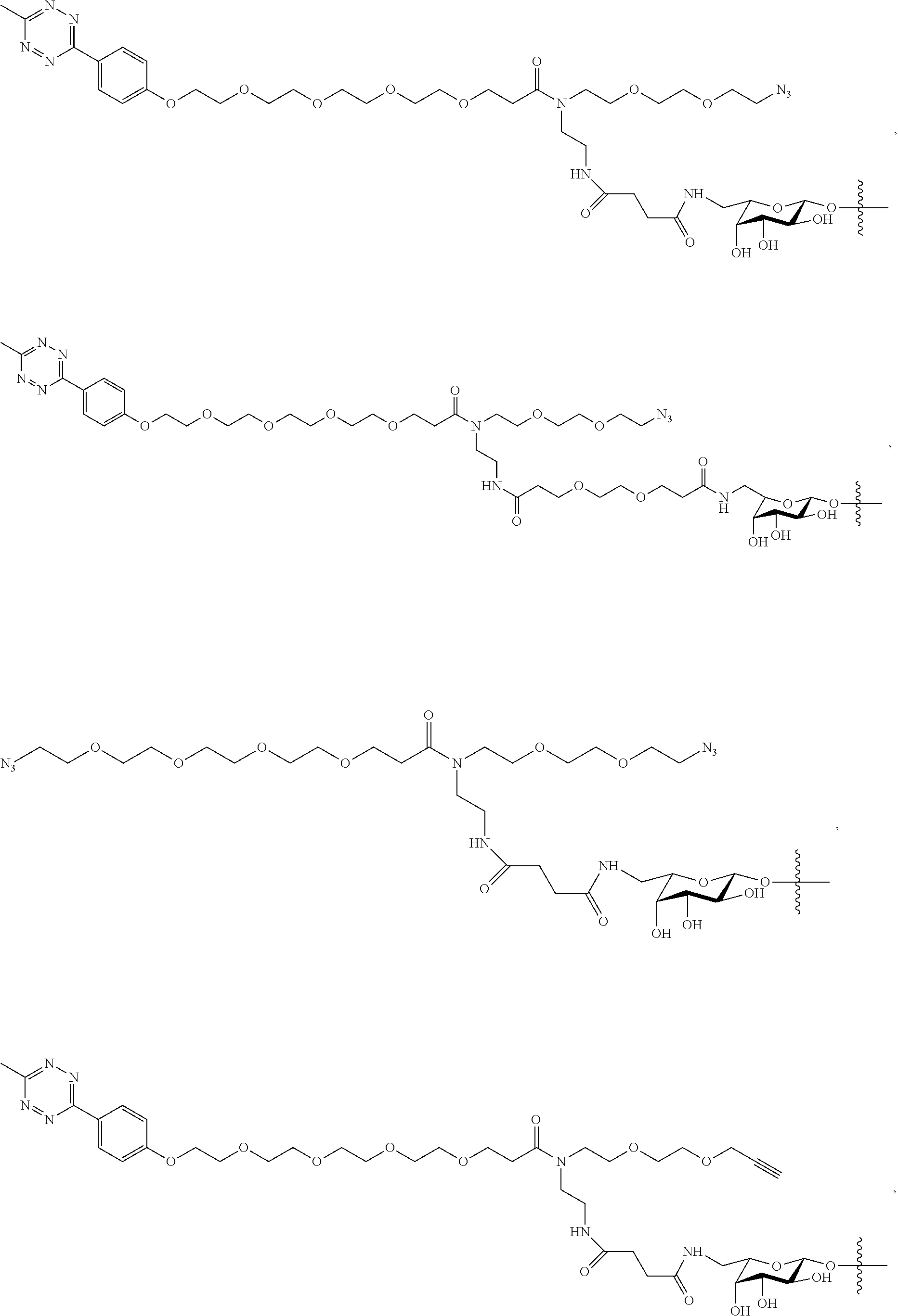

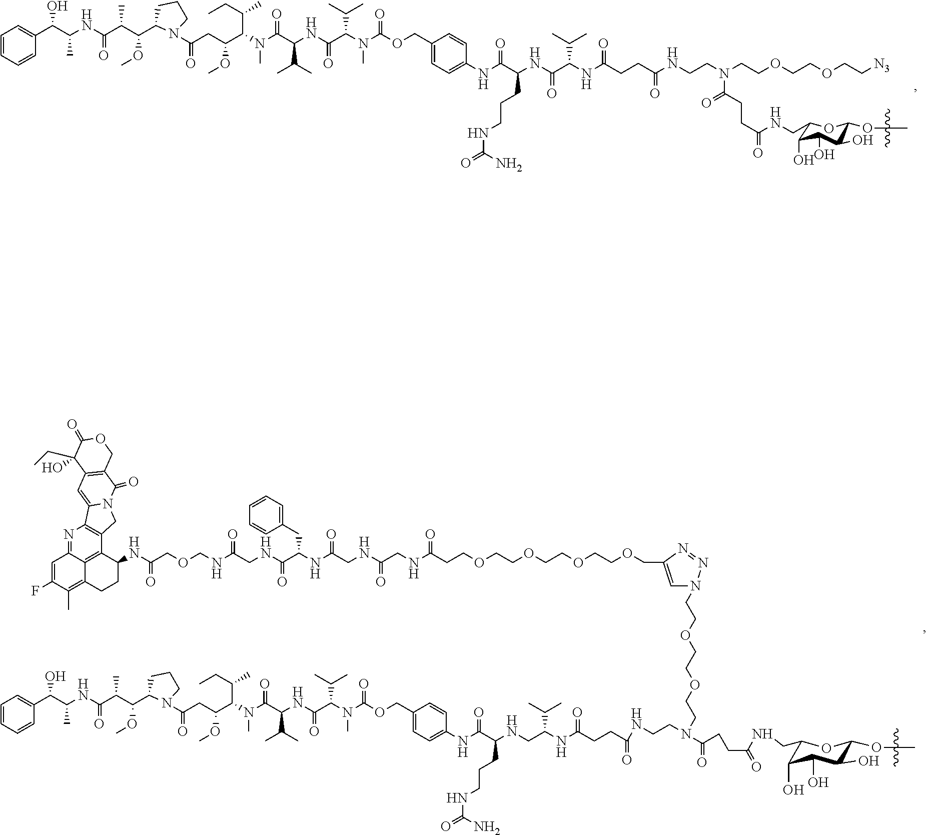

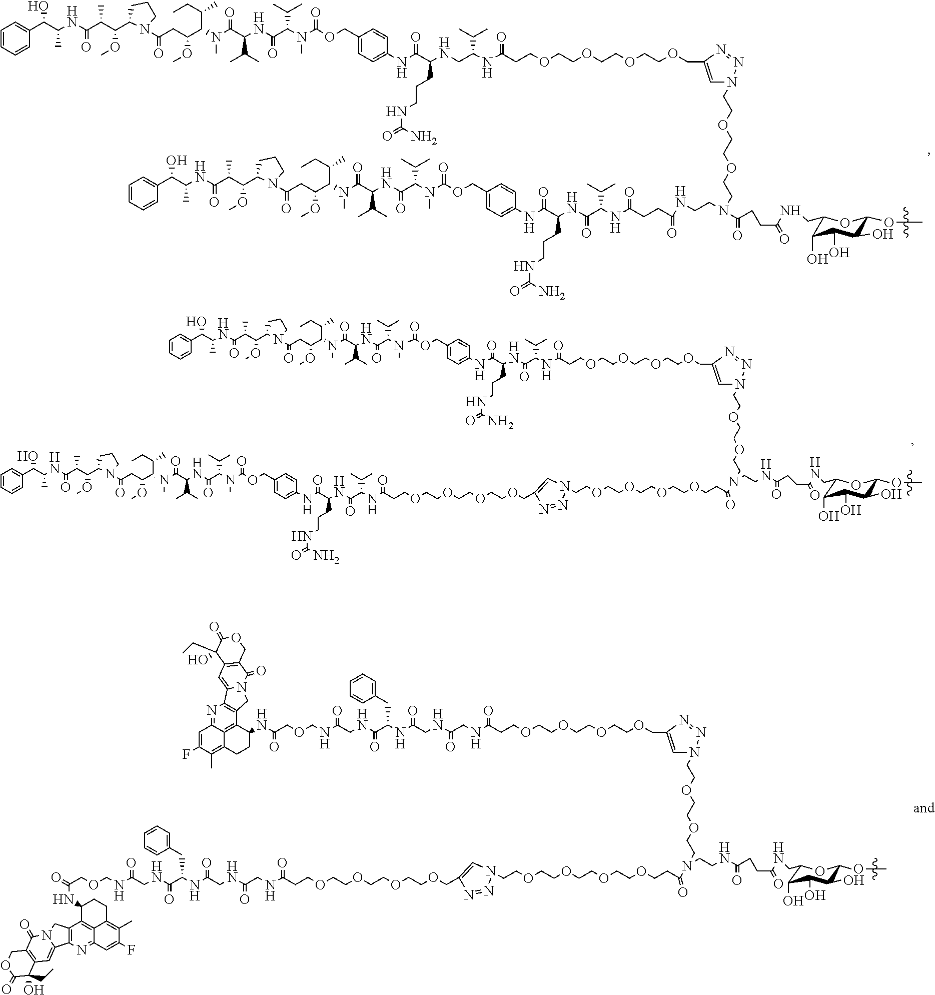

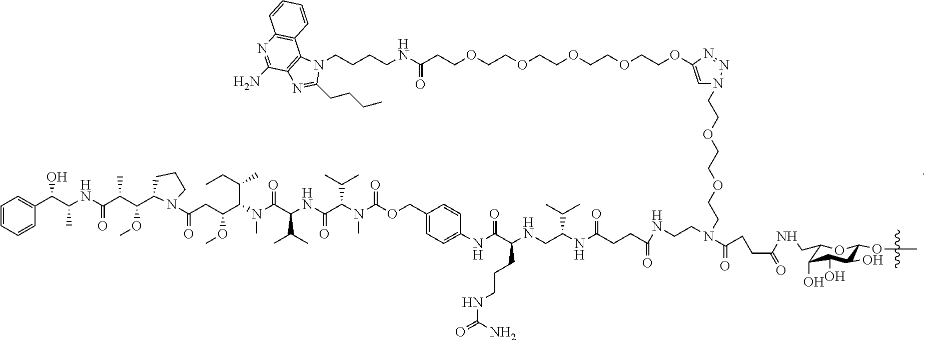

70-73. (canceled)

74. A method for preparing a protein conjugate, comprising step (a):
contacting a fucose derivative donor Q-Fuc* with a protein comprising an oligosaccharide in the presence of a catalyst, wherein said oligosaccharide comprises Formula (VII): -GlcNAc(Fuc)_{b}-GalX (VII), to obtain a protein conjugate comprising the structure of Formula (I): 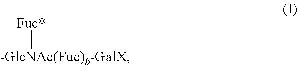
wherein:
said GlcNAc is directly or indirectly linked to an amino acid of said protein; and said protein comprises an antigen binding fragment and/or an Fc fragment;
said GalX is an optionally substituted galactose;
said Fuc is a fucose, and b is 0 or 1;
said Q is uridine diphosphate (UDP), a guanosine diphosphate (GDP) or a cytidine diphosphate (CDP); and
said Fuc* comprises the structure of Formula (II): 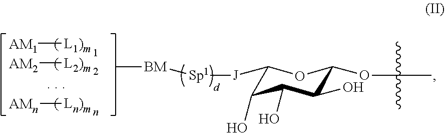
wherein
J has a structure of 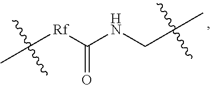
wherein R_{f} is —CH₂—, —NH— or —O—, and the right side of the structure is directly linked to the 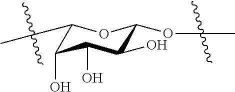
of Formula (II);
Sp¹ is selected from the group consisting of: 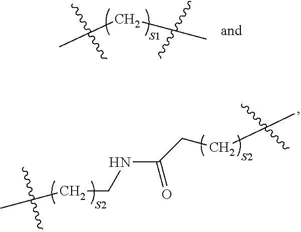
said S1 is independently an integer from 1-50, said S2 is independently an integer from 0-50, each said —CH₂— is independently optionally replaced by —O— with the proviso that two or more consecutive —CH₂— are not simultaneously replaced by —O—, the right side of the structure is linked to said J and the left side of the structure is linked to said BM, and d is 0 or 1:
BM comprises 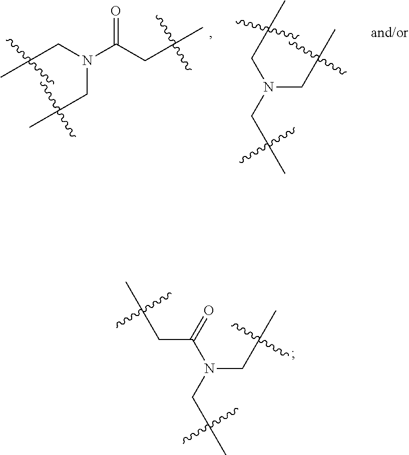 [SMILES: *CC(=O)N(C*)C* ; *CN(C*)C* ; *CC(=O)N(C*)C*]
each of L₁ to Lₙ is independently a linker of Formula (IV): (CL)ᵥ-(FL)ₓ (IV), wherein
FL is a spacer moiety selected from the group consisting of: 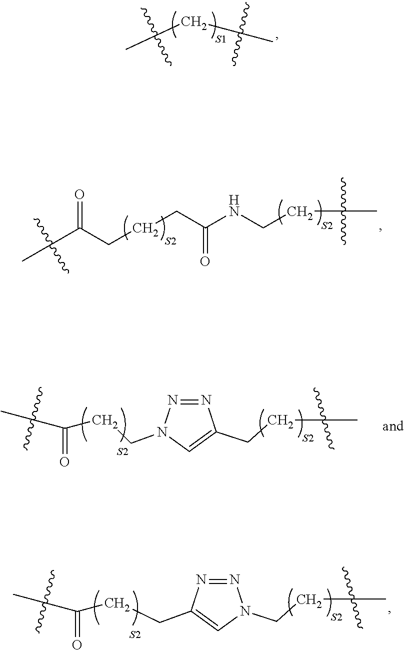
wherein said S1 is an integer from 1-50, each said S2 is independently an integer from 0-50, each said —CH₂— is independently optionally replaced by —O— with the proviso that two or more consecutive —CH₂— are not simultaneously replaced by —O—, the right side of the structure is linked to said BM, and the left side of the structure is linked to said CL or each of said AM₁ to AMₙ, and x is 0 or 1;
CL is a cleavable linker, and CL is an acid-labile linker, a redox-active linker, a photo-active linker and/or a proteolytically cleavable linker, and y is 0 or 1;
the right side of Formula (IV) is linked to said BM and the left side of Formula (IV) is linked to said AM:
m₁ to mₙ each independently is 0 or 1;
AM₁ to AMₙ each independently is chemically active molecule, an enzymatically active molecule, a biologically active molecule, and/or a pharmaceutically active molecule;
n is an integer from 2-10; and
said GalX is linked to said GlcNAc through a β1,4 linkage;
said Fuc* is linked to said GlcNAc through an α1,3 linkage; and,
n is 2, and said Fuc* comprises the structure of Formula (III):

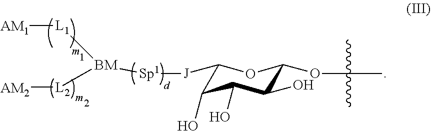

75-76. (canceled)

77. The method of claim 74, wherein said catalyst comprises a fucosyltransferase and comprises an amino acid sequence as set forth in any of SEQ ID NO: 16-25.

78-102. (canceled)

103. The method of claim 74, wherein said AM₁ to AMₙ independently comprises a chemically active molecule or enzymatically active molecule X_{F} and X_{F} comprises a functional moiety selected from the group consisting of: 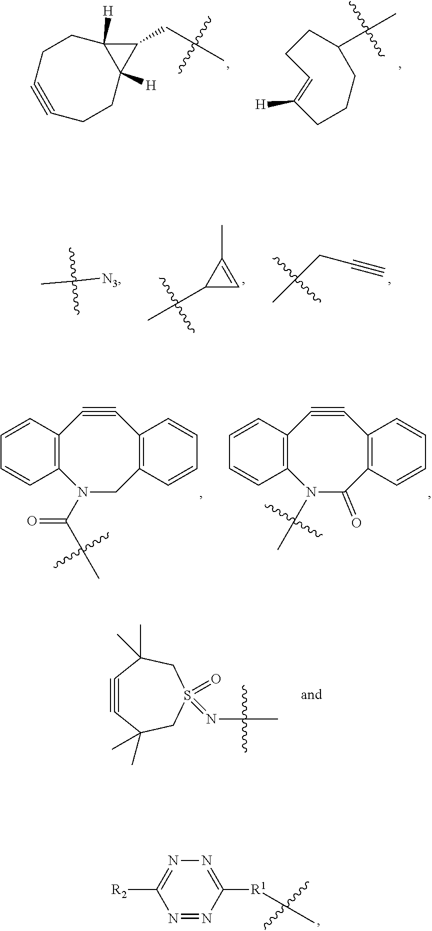
wherein R₁ is selected from the group consisting of C₁-C₁₀ alkylene group, C₅-C₁₀ (hetero)arylene group, C₆-C₁₀ alkyl(hetero)arylene group and C₆-C₁₀ (hetero)arylalkylene group, and R₂ is selected from the group consisting of hydrogen, C₁-C₁₀ alkyl group, C₅-C₁₀ (hetero)aryl group, C₅-C₁₀ alkyl(hetero)aryl group and C₅-C₁₀ (hetero)arylalkyl group,
and/or
said AM₁ to AMₙ independently comprises a biologically active molecule and/or pharmaceutically active molecule P_{F} and P_{F} comprises a cytotoxin, an agonist, an antagonist, an antiviral agent, an antibacterial agent, a radioisotope or a radionuclide, a metal chelator, a fluorescent dye, a biotin, an oligonucleotide, a polypeptide, or any combination thereof.

104-115. (canceled)

116. The method of claim 103, wherein said P_{F} comprises a MMAE, a DXd, T785 and/or derivatives thereof.

117. (canceled)

118. The method of claim 74, wherein said GalX is a galactose, or GalX is a substituted galactose and the hydroxyl group at one or more positions selected from the C2 position, the C3 position, the C4 position and the C6 position of the galactose, is substituted; and said GalX is a monosaccharide.

119-128. (canceled)

129. The method of claim 74, wherein said GalX is selected from the group consisting of

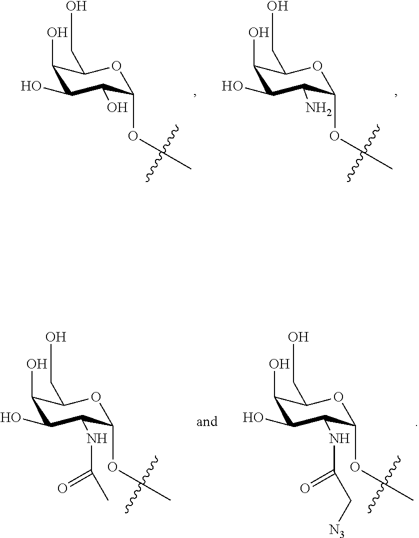

130-149. (canceled)

150. The method of claim 74, wherein said Q-Fuc* is of a structure selected from the followings:

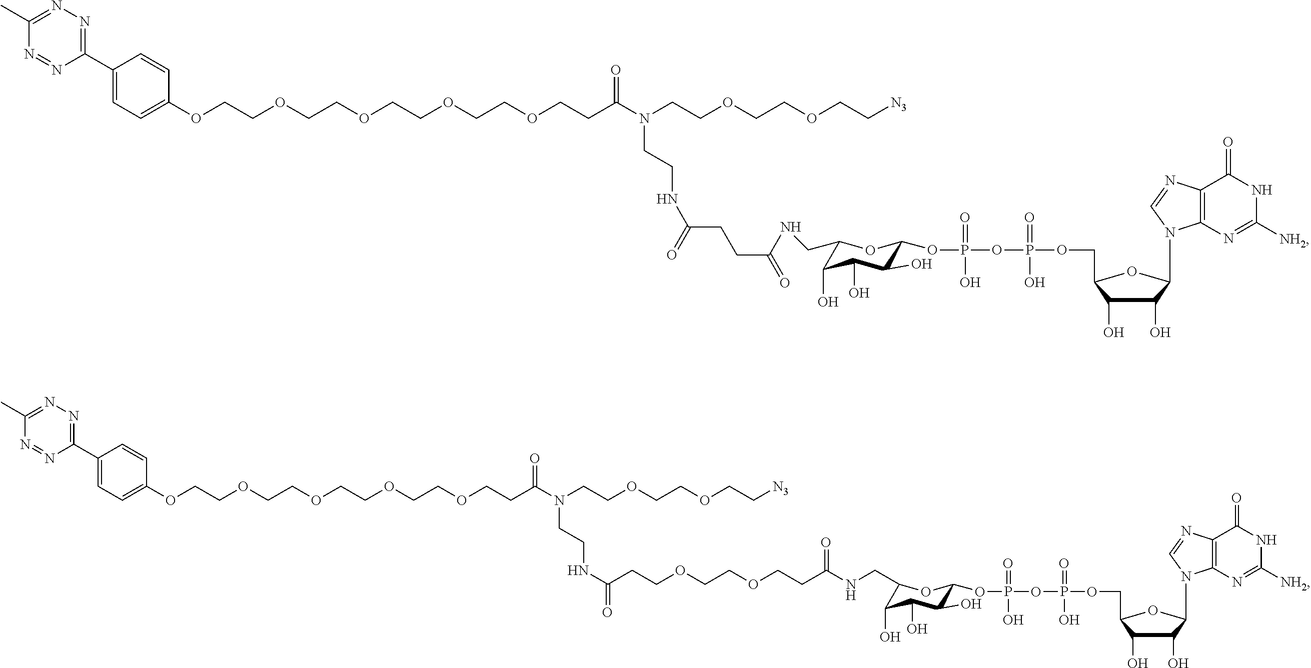

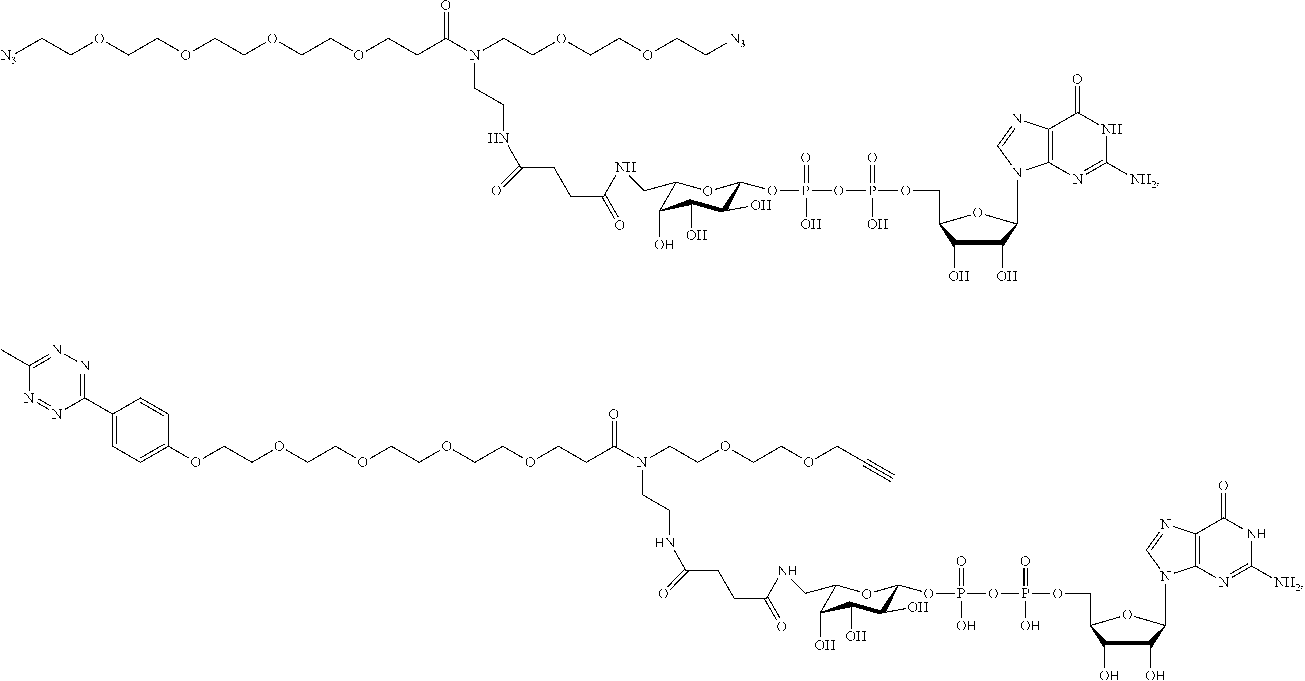

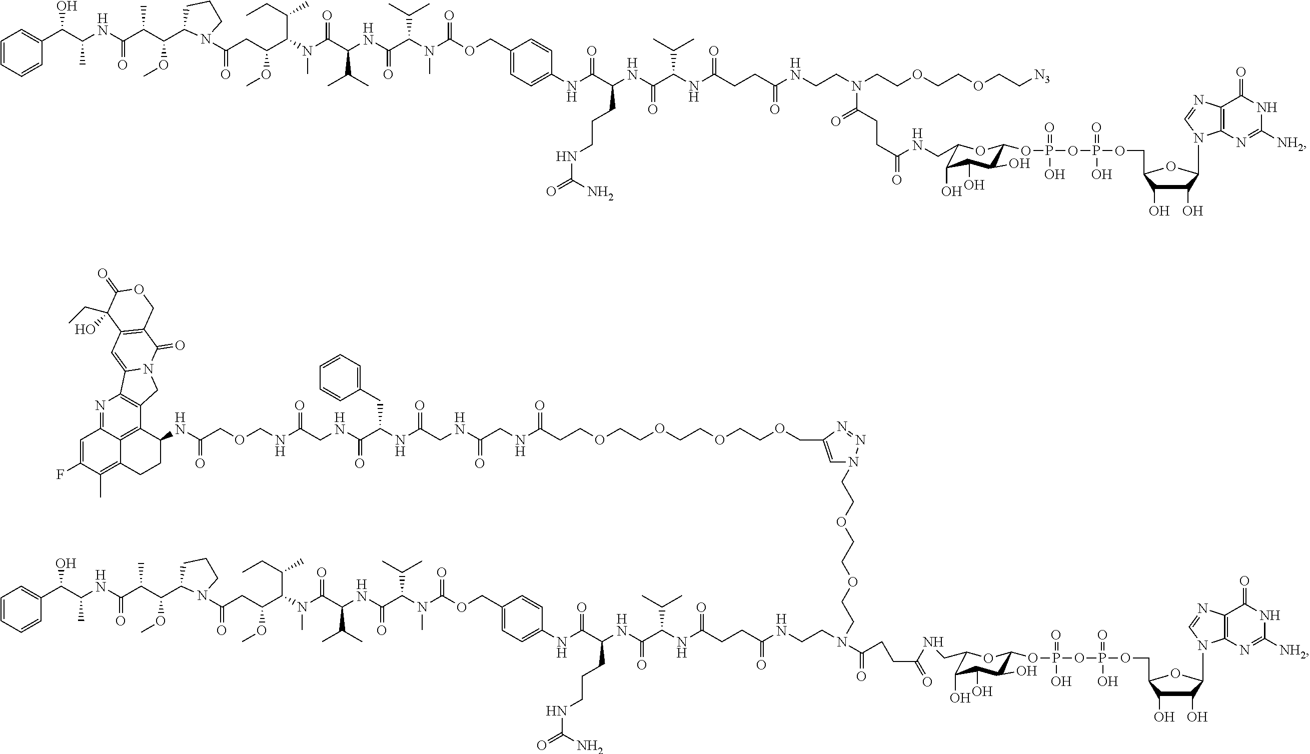

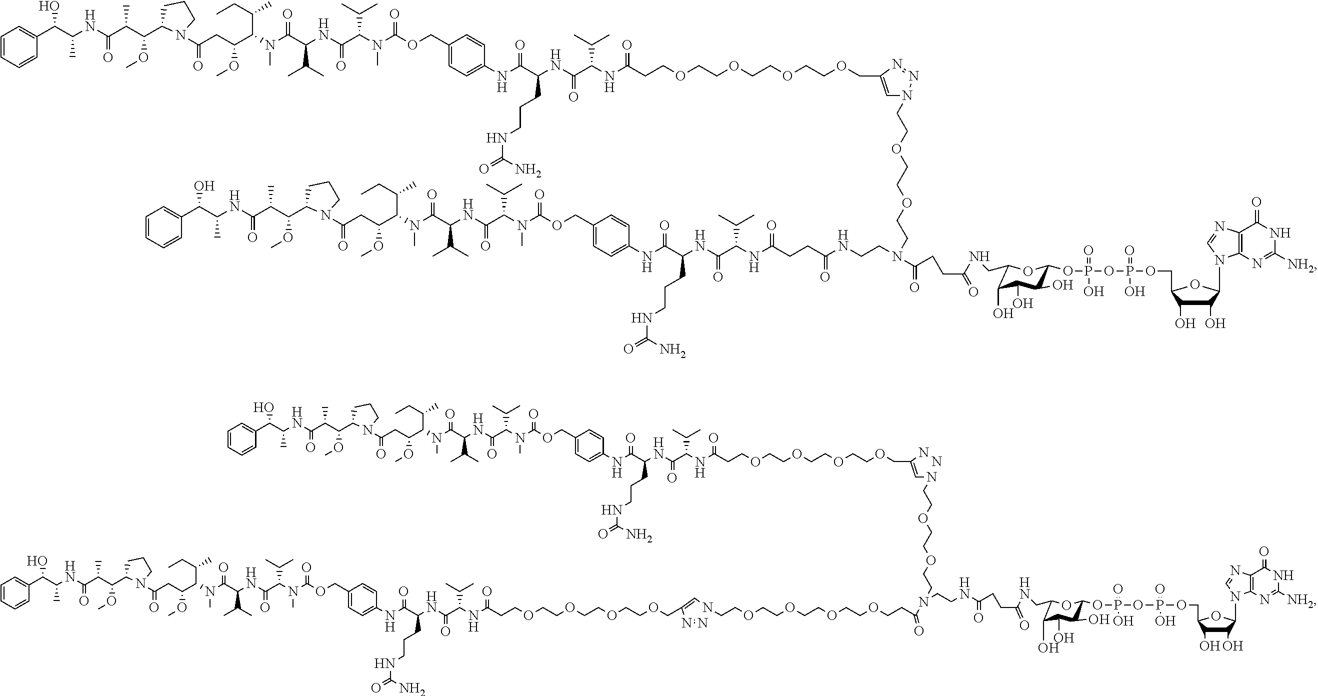

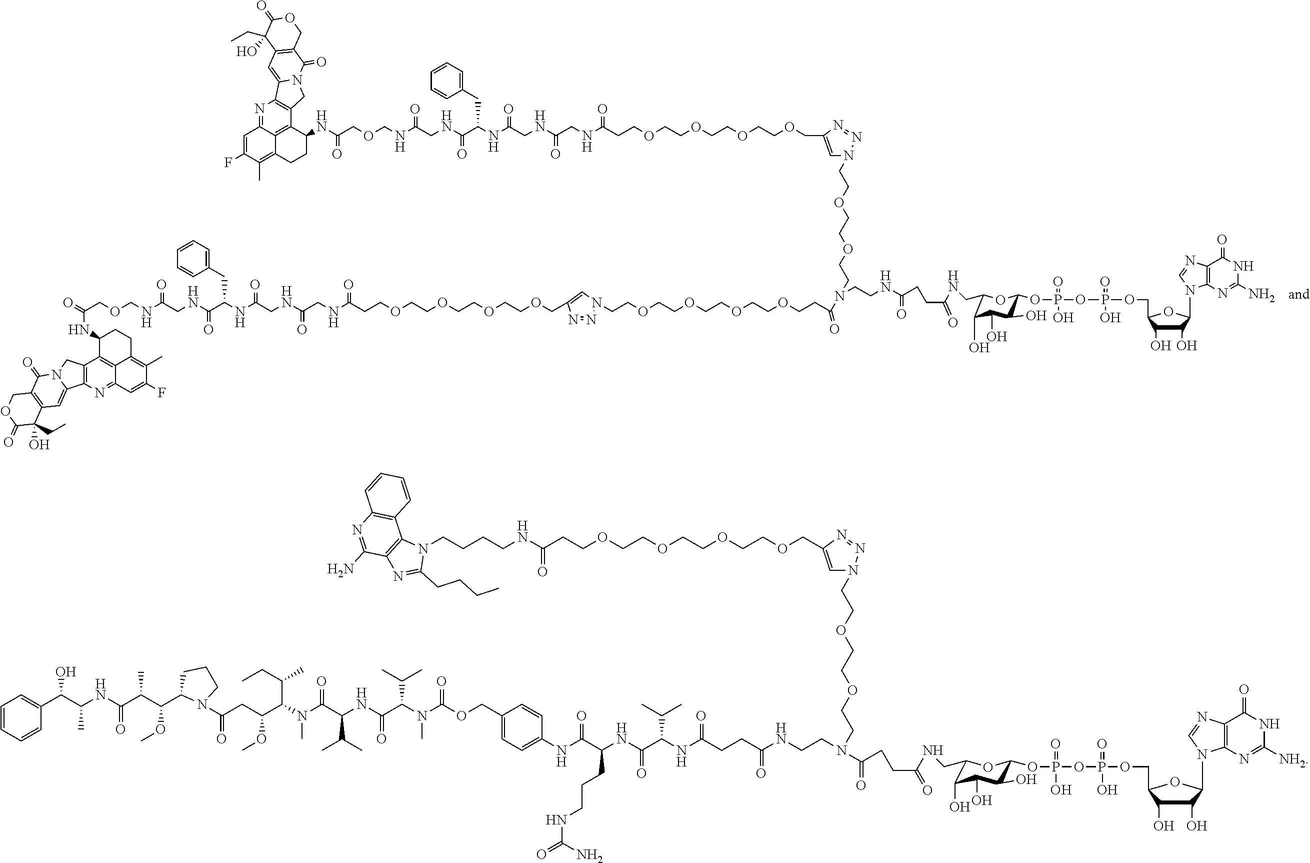

151-153. (canceled)

154. The method of claim 74, wherein said protein comprising the oligosaccharide comprises a structure of Formula (VIII) 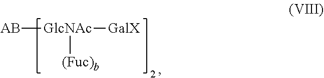
wherein said AB is an antibody comprising a Fc fragment or a Fc-fusion protein, the GlcNAc is directly linked to an Asn of the Fc fragment of the AB, the Fuc is linked to the GlcNAc through an α1,6 linkage, the GalX is linked to the GlcNAc through a β1,4 linkage, and b is 0 or 1.

155. The method of claim 154, further comprising the steps of:
i) modifying a glycosylated antibody comprising the Fc fragment or the Fc-fusion protein with an endoglycosidase to obtain a modified protein; and
ii) contacting the modified protein with a UDP-GalX in the presence of a catalyst to obtain said protein comprising the structure of Formula (VIII);
said b is 0 or 1.

156. The method of claim 154, further comprising the steps of:
i) modifying a glycosylated antibody comprising an Fc fragment or the Fc-fusion protein with an endoglycosidase and an α1,6 fucosidase to obtain a modified protein; and
ii) contacting the modified protein with a UDP-GalX in the presence of a catalyst to obtain said protein comprising the structure of Formula (VIII);
said b is 0.

157. (canceled)

158. The method of claim 74, wherein said protein comprises the structure of Formula (IX): 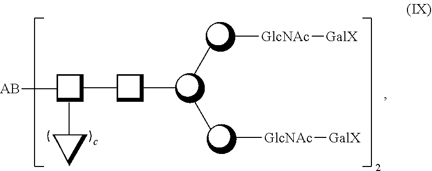
wherein said AB is an antibody comprising a Fc fragment or a Fc-fusion protein, [symbol] is a GlcNAc, [symbol] is a mannose, [symbol] is a fucose to the [symbol] through a α1,6 linkage, c is 0 or 1;
said oligosaccharide is linked to an Asn of the Fc fragment of the AB through the [symbol] , and
the GalX is linked to the GlcNAc through a β1,4 linkage.

159. (canceled)

160. The method of claim 158, further comprising contacting an antibody comprising an Fc fragment or the Fc-fusion protein having a glycoform of G₀(F)_{0,1}, G₁(F)_{0,1} and/or G₂(F)_{0,1} with a UDP-GalX in the presence of a catalyst, to obtain said protein comprising the structure of Formula (IX).

161-164. (canceled)

165. A composition, comprising the protein conjugate of claim 1.

166-169. (canceled)

170. A method for preventing or treating a disease, comprising administrating the protein conjugate of claim 1.

171. (canceled)
